(19) United States
(12) Patent Application Publication
(10) Pub. No.: US 2007/0124218 A1
(43) Pub. Date: May 31, 2007

(54) Computational and/or control systems related to individualized nutraceutical selection and packaging

(75) Inventors: Edward K.Y. Jung, Bellevue, WA (US); Royce A. Levien, Lexington, MA (US); Robert W. Lord, Seattle, WA (US); Mark A. Malamud, Seattle, WA (US); John D. Rinaldo JR., Bellevue, WA (US); Lowell L. Wood JR., Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/478,296

(22) Filed: Jun. 28, 2006

Related U.S. Application Data
(63) Continuation-in-part of application No. 11/453,571, filed on Jun. 14, 2006.
(63) Continuation-in-part of application No. 11/478,341, filed on Jun. 28, 2006.
(63) Continuation-in-part of application No. 11/474,109, filed on Jun. 23, 2006.
(63) Continuation-in-part of application No. 11/314,945, filed on Dec. 20, 2005.
(63) Continuation-in-part of application No. 11/291,482, filed on Nov. 30, 2005.

(51) Int. Cl. G06Q 10/00

(52) U.S. Cl. 705/28

(57) ABSTRACT

The present disclosure relates to methods and systems that may be used with nutraceutical agents.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation of U.S. patent application Ser. No. 11/453,571, entitled INDIVIDUALIZED PHARMACEUTICAL SELECTION AND PACKAGING, naming Edward K. Y. Jung, Royce A. Levien, Robert W. Lord, Mark A. Malamud, John D. Rinaldo, Jr., and Lowell L. Wood, Jr. as inventors, filed 14 Jun. 2006.

For purposes of the USPTO extra-statutory requirements, the present application is related to United States Patent Application No. Not Yet Assigned, Attorney Docket No. 0306-002-033, entitled, COMPUTATIONAL AND/OR CONTROL SYSTEMS RELATED TO INDIVIDUALIZED NUTRACEUTICAL SELECTION AND PACKAGING, naming Edward K. Y. Jung, Royce A. Levien, Robert W. Lord, Mark A. Malamud, John D. Rinaldo, Jr., and Lowell L. Wood Jr. as inventors, filed 28 Jun. 2006, herein incorporated by reference to the extent such subject matter is not inconsistent herewith.

For purposes of the USPTO extra-statutory requirements, the present application is related to United States Patent Application No. Not Yet Assigned, Attorney Docket No. 0306-002-009, entitled CUSTOMIZED VISUAL MARKING FOR MEDICATION LABELING, naming Edward K. Y. Jung, Royce A. Levien, Robert W. Lord, Mark A. Malamud, John D. Rinaldo, Jr., and Lowell L. Wood Jr. as inventors, filed 23 Jun. 2006, herein incorporated by reference to the extent such subject matter is not inconsistent herewith.

For purposes of the USPTO extra-statutory requirements, the present application is related to U.S. patent application Ser. No. 11/314,945, entitled GENERATING A REQUEST FROM A NUTRACEUTICAL INVENTORY, naming Edward K. Y. Jung, Royce A. Levien, Robert W. Lord, Mark A. Malamud, John D. Rinaldo, Jr., Clarence T. Tegreene, and Lowell L. Wood Jr. as inventors, filed 20 Dec. 2005, herein incorporated by reference to the extent such subject matter is not inconsistent herewith.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present applicant entity has provided above a specific reference to the application(s)from which priority is being claimed as recited by statute. Applicant entity understands that the statute is unambiguous in its specific reference language and does not require either a serial number or any characterization, such as “continuation” or “continuation-in-part,” for claiming priority to U.S. patent applications. Notwithstanding the foregoing, applicant entity understands that the USPTO's computer programs have certain data entry requirements, and hence applicant entity is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present disclosure relates methods and systems that may be used with nutraceutical agents.

### SUMMARY

In some embodiments a method is provided that includes accepting input of one or more parameters associated with an individual, selecting two or more nutraceutical agents in response to at least one of the one or more parameters associated with the individual, and packaging the two or more nutraceutical agents in response to at least one of the one or more parameters associated with the individual. In addition to the foregoing, other method aspects are described in the claims, drawings, and text forming a part of the present disclosure.

In some embodiments a system is provided that includes circuitry for accepting input of one or more parameters associated with an individual, circuitry for selecting two or more nutraceutical agents in response to at least one of the one or more parameters associated with the individual, and circuitry for packaging the two or more nutraceutical agents in response to at least one of the one or more parameters associated with the individual. In addition to the foregoing, other system aspects are described in the claims, drawings, and text forming a part of the present disclosure.

In some embodiments a system is provided that includes means for accepting input of one or more parameters associated with an individual, means for selecting two or more nutraceutical agents in response to at least one of the one or more parameters associated with the individual, and means for packaging the two or more nutraceutical agents in response to at least one of the one or more parameters associated with the individual. In some embodiments, such means include but are not limited to circuitry and/or programming for effecting the herein-referenced functional aspects; the circuitry and/or programming can be virtually any combination of hardware, software, and/or firmware configured to effect the herein-referenced functional aspects depending upon the design choices of the system designer. In addition to the foregoing, other system aspects means are described in the claims, drawings, and/or text forming a part of the present application.

In addition to the foregoing, other system aspects are described in the claims, drawings, and text forming a part of the present disclosure.

In some embodiments a system is provided that includes a signal-bearing medium bearing at least one of: one or more instructions for accepting input of one or more parameters associated with an individual; one or more instructions for selecting two or more nutraceutical agents in response to at least one of the one or more parameters associated with the individual; and one or more instructions for packaging the two or more nutraceutical agents in response to at least one of the one or more parameters associated with the individual. In addition to the foregoing, other system aspects are described in the claims, drawings, and text forming a part of the present disclosure.

In some embodiments, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer. In addition to the foregoing, other system aspects are described in the claims, drawings, and/or text forming a part of the present application.

The foregoing summary is illustrative only and is not intended to be in any way limiting. In addition to the illustrative aspects, embodiments, and features described above, further aspects, embodiments, and features will become apparent by reference to the drawings, claims, and the following detailed description.

### BRIEF DESCRIPTION OF THE FIGURES

FIG. 1 illustrates an example system 100 in which embodiments may be implemented.

FIG. 2 illustrates an operational flow representing example operations related to methods for individualized nutraceutical selection and packaging.

FIG. 3 illustrates alternative embodiments of the example operation flow of FIG. 2.

FIG. 4 illustrates alternative embodiments of the example operation flow of FIG. 2.

FIG. 5 illustrates alternative embodiments of the example operation flow of FIG. 2.

FIG. 6 illustrates alternative embodiments of the example operation flow of FIG. 2.

FIG. 7 illustrates alternative embodiments of the example operation flow of FIG. 2.

FIG. 8 illustrates alternative embodiments of the example operation flow of FIG. 2.

FIG. 9 illustrates alternative embodiments of the example operation flow of FIG. 2.

FIG. 10 illustrates an example system 1000 in which embodiments may be implemented.

FIG. 11 illustrates alternative embodiments of the system of FIG. 10.

FIG. 12 illustrates alternative embodiments of the system of FIG. 10.

FIG. 13 illustrates alternative embodiments of the system of FIG. 10.

FIG. 14 illustrates alternative embodiments of the system of FIG. 10.

FIG. 15 illustrates alternative embodiments of the system of FIG. 10.

FIG. 16 illustrates alternative embodiments of the system of FIG. 10.

FIG. 17 illustrates alternative embodiments of the system of FIG. 10.

FIG. 18 illustrates an example system 1800 in which embodiments may be implemented.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

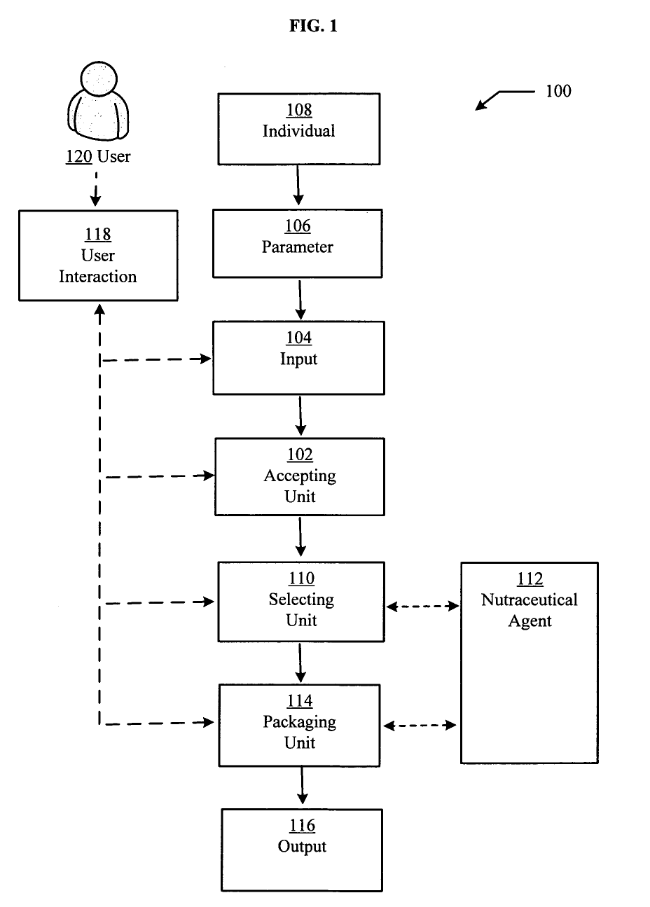

FIG. 1 illustrates an example system 100 in which embodiments may be implemented. In some embodiments, the system 100 is operable to provide a method and system for individualized nutraceutical selection and packaging. In some embodiments, one or more accepting units 102 accept input 104 of one or more parameters 106 associated with an individual 108, one or more selecting units 110 may then select two or more nutraceutical agents 112 in response to at least one of the one or more parameters 106 associated with the individual 108, and one or more packaging units 114 may then package the two or more nutraceutical agents 112 in response to at least one of the one or more parameters 106 associated with the individual 108. In some embodiments, the two or more nutraceutical agents 112 may be packaged and output 116 in an administration form that may be administered to an individual 108. In some embodiments, the system provides for user interaction 118 with a user 120. In some embodiments, one or more users 120 may provide input 104 to one or more accepting units 102. In some embodiments, one or more users 120 may interact with one or more accepting units 102. In some embodiments, one or more users 120 may interact with one or more selecting units 110. In some embodiments, one or more users 120 may interact with one or more packaging units 114. In some embodiments, one or more users 120 may interact with one or more accepting units 102, one or more selecting units 110, one or more packaging units 114, and/or substantially any combination thereof. In some embodiments, the individual units may be combined together into a single system 100. For example, in some embodiments, the accepting unit 102, selecting unit 110, and packaging unit 114 may all be combined into a single system 100. In some embodiments, the individual units maybe located in separate locations. For example, an accepting unit 102 may be located in one area, a selecting unit 110 may be located in another area, and a packaging unit 114 may be located in yet another area. For example, in some embodiments, an accepting unit 102 may be in the form of a personal digital assistant into which an individual 108 can input 104 parameters 106 associated with the individual 108. A separately located selecting unit 110 may receive information from the accepting unit 102 and select two or more nutraceutical agents 112 in response to the one or more parameters 106 associated with the individual 108. A separately located packaging unit 114 may receive information from the selecting unit 110 and package two or more nutraceutical agents 112 in response to the one or more parameters 106 associated with the individual 108. Accordingly, the individual units of the system 100 described in FIG. 1 may be oriented in substantially any physical combination. Such systems 100 may be located in numerous areas. Examples of such areas include, but are not limited to, hospitals, clinics, physician's offices, dentist's offices, pharmacies, homes, nutraceutical companies, pharmaceutical companies, veterinary clinics, stores (i.e., health-food stores, food supplement stores, sporting goods stores, grocery stores, and the like), pet-owners homes, gyms, and the like.

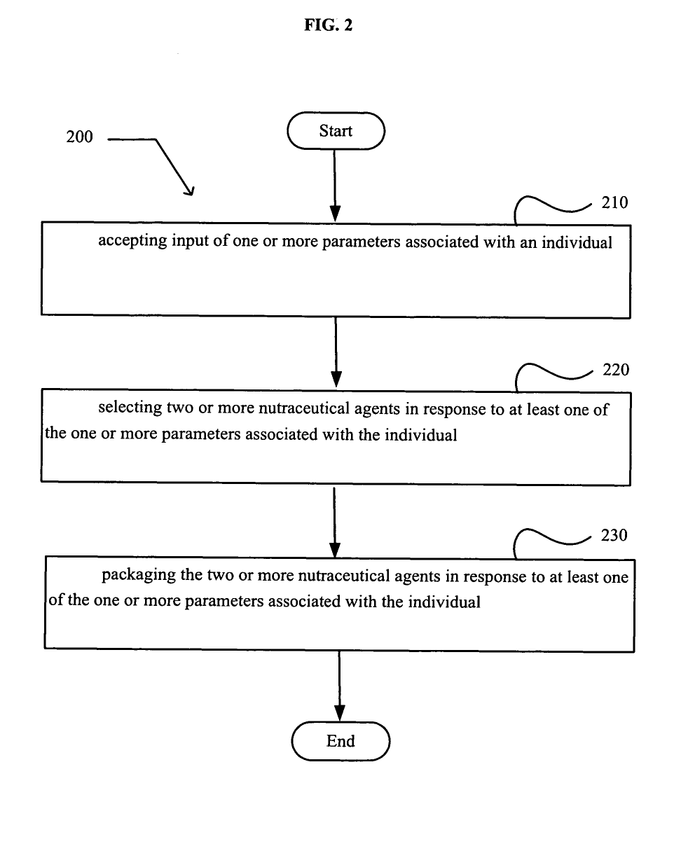

FIG. 2 illustrates an operational flow 200 representing examples of operations that are related to the performance of a method for individualized nutraceutical selection and packaging. In FIG. 2 and in following figures that include various examples of operations used during performance of the method, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 200 includes an accepting operation 210 involving accepting input of one or more parameters associated with an individual. In some embodiments, one or more accepting units 102 may accept input 104 of one or more parameters 106 associated with an individual 108.

In some embodiments, an individual 108 may be a human. In some embodiments, an individual 108 may be a non-human animal. Examples of such non-human animals include, but are not limited to, domestic pets such as dogs, cats, horses, potbelly pigs, ferrets, rodents, reptiles, amphibians, and the like. Non-human animals also include animals that include, but are not limited to, cattle, sheep, goats, chickens, pigs, and the like. Accordingly, the systems and methods described herein may be used in association with substantially any human and/or non-human animal.

Numerous parameters 106 may be associated with an individual 108. Such parameters 106 may include, but are not limited to, physical characteristics, metabolic characteristics, financial characteristics, and the like. Examples of parameters 106 include, an individual's height, weight, gender, kidney function, liver function, level of physical fitness, age, allergic response, metabolic level (i.e., resting metabolic rate and/or activity-related metabolic rate), disease state, body fat percentage, personal health habits (i.e., smoking, alcohol consumption, diet, illegal drug use, and the like), family health history, insurance coverage, food supplement usage, nutraceutical usage, non-prescription drug use, prescription drug use, pregnancy status, and the like. In some embodiments, the one or more parameters 106 may be specifically associated with an individual 108. As such, in some embodiments, the one or more parameters 106 may be unique to the individual 108 as opposed to being common to a group. For example, in some embodiments, an individual 108 may be a member of a group of persons who are diabetic while exhibiting one or more parameters 106, such as metabolic characteristics, that are unique to the individual 108. Accordingly, in some embodiments, one or more parameters 106 may be input that provide for selection of nutraceutical agents 112 in accordance with one or more parameters 106 that are specifically associated with an individual 108.

Numerous technologies may be used to provide input 104 that include one or more parameters 106 associated with an individual 108. Examples of such technologies include, but are not limited to, hardwired input 104, wireless input 104, computer input 104, telephonic input 104, internet based input 104, intranet based input 104, digital input 104, analog input 104, input 104 from a human, input 104 from a palm held organizer, input 104 from a personal digital assistant, input 104 from a web enabled cellular telephone, and the like. In some embodiments, one or more accepting units 102 accept input 104 from one source. In some embodiments, one or more accepting units 102 accept input 104 from more than one source. For example, in some embodiments, an accepting unit 102 may accept input 104 from an insurance company, a physician, a pharmacist, a clinical laboratory, a pharmaceutical company, and a nutraceutical company. In some embodiments, input 104 may be associated with a physician input 104, a pharmacist input 104, a patient input 104, a machine input 104 and/or substantially any combination thereof.

In some embodiments, an accepting unit 102 may include an input device. For example, in some embodiments, an accepting unit 102 may include an interface, such as a keyboard, touch-screen and/or the like, where parameters 106 associated with an individual 108 may be input 104 directly into the accepting unit 102. In some embodiments, an accepting unit 102 may lack an interface where parameters 106 associated with an individual 108 may be directly input 104 into the accepting unit 102. In some embodiments, an accepting unit 102 may accept input 104 of one or more parameters 106 associated with an individual 108 from one or more locations that are remote from the accepting unit 102. For example, in some embodiments, an accepting unit 102 may accept input 104 from a wireless device, the internet, an intranet, a telephone, a palm held organizer, input 104 from a personal digital assistant, input 104 from a web enabled cellular telephone, and the like.

After a start operation, the operational flow 200 includes a selecting operation 220 involving selecting two or more nutraceutical agents in response to at least one of the one or more parameters associated with the individual. In some embodiments, one or more selecting units 110 may select two or more nutraceutical agents 112 in response to at least one of the one or more parameters 106 associated with the individual 108.

In some embodiments, one or more selecting units 110 act to select two or more nutraceutical agents 112 in response to at least one of the one or more parameters 106 associated with an individual 108. In some embodiments, one or more selecting units 110 may select one or more first nutraceutical agents 112 in response to at least one of the one or more parameters 106 associated with an individual 108 and select one or more second nutraceutical agents 112 based on the identity of the one or more first nutraceutical agents 112 selected. For example, in some embodiments, one or more selecting units 110 may select the first and second nutraceutical agents 112 to act synergistically with each other when administered to an individual 108. In some embodiments, one or more selecting units 110 may select the first and second nutraceutical agents 112 so that they do not contraindicate each other when administered to an individual 108. Nutraceutical agents 112 may be selected in response to numerous parameters 106.

Nutraceuticals typically include natural, bioactive chemical compounds or any substance that is a plant, food, or an extracted part of a food, that provides medical or health benefits but which generally fall outside regulations controlling pharmaceuticals. Included in this category of substances may be foods, isolated nutrients, supplements and herbs. Nutraceuticals are often referred to as phytochemicals or functional foods and include dietary supplements. Numerous nutraceuticals have been described (i.e., Roberts et al., Nutraceuticals: The Complete Encyclopedia of Supplements, Herbs, Vitamins, and Healing Foods, 1^{st}Edition, Perigee Trade (2001) and Susan G. Wynn, Emerging Therapies: Using Herbs and Nutraceuticals for Small Animals, American Animal Hospital Assn Press (1999)). Examples of nutraceuticals include, but are not limited to, Amino Acids, Terpenoids, Carotenoid Terpenoids (Lycopene, Beta-Carotene, Alpha-Carotene, Lutein, Zeaxanthin, Astaxanthin), Non-Carotenoid Terpeniods (Perillyl Alcohol, Saponins, Terpeneol, Terpene Limonoids), Polyphenolics, Flavonoid Polyphenolics (Anthocyanins, Catechins, Isoflavones, Hesperetin, Naringin, Rutin, Quercetin, Silymarin, Tangeretin, Tannins), Phenolic Acids (Ellagic Acid, Chlorogenic Acid, Para-Coumaric Acid, Phytic Acid, Cinnamic Acid), Other Non-Flavonoid Polyphenolics (Curcumin, Resveratrol, Lignans), Glucosinolates, Isothiocyanates (Phenethyl Isothiocyanate, Benzyl Isothiocyanate, Sulforaphane), Indoles (Indole-3-Carbinol (13C), Thiosulfonates, Phytosterols (Beta-Sitosterol), Anthraquinones (Senna, Barbaloin, Hypericin), Capsaicin, Piperine, Chlorophyll, Betaine, Pectin, Oxalic Acid, Acetyl-L-Carnitine, Allantoin, Androsterondiol, Androsterondione, Betaine (Trimethylglycine), Caffeine, Calcium pyvurate (Pyruvic Acid), Carnitine, Carnosine, Carotene (alpha & beta), Carotenoid (Total for beadlets), Choline, Chlorogenic Acid, Cholic Acid (Ox Bile), Chondroitin Sulfate, Chondroitin Sulfate (Total Mucopolysaccharides), Cholestin, Chrysin, Coenzyme Q10 (Co-Q10), Conjugated Linoleic Acid (CLA), Corosolic Acid, Creatine, Dehydroepiandrosterone (DHEA), Dichlorophen, Diindolymethane (DIM), Dimethyglycine (DMG), Dimercapto Succinic Acid (DMSA), Ebselen, Ellagic Acid, Fisetin, Formonetin, Glucaric Acid (Glucarate), Glucosamine (HCl or Sulfate), Glucosamine (N-Acetyl), Glutathione (Reduced), Hesperidine, Hydroxy-3-Methylbutyric Acid (HMB), 5-Hydroxytryptophan (L-5-HTP), Indole-3-Carbinol, Inositol, Isothiocyanates, Linolenic Acid-Gamma (GLA), Lipoic Acid (alpha), Lutein, Lycopene, Melatonin, Methylsulfonylmethane (MSM), Naringin, Pancreatin, Para-aminobenzoic Acid (PABA), Paraben (methyl or propyl), Phenolics, Phosphatidylcholine (Lecithin), Phosphatidylserine, Phospholipids, Phytosterols, Pregersterone, Pregnenolone, Quercetin, Resveratrol, D-Ribose, Rutin, S-adenosylmethionine (SAM-e), Salicylic Acid, Sulforaphane, Tartaric Acid, Taxifolin, Tetrahydropalmatine, Thephyline, Theobromine, Tigogenin, Troxerutin, Tryptophan, Tocotrienol (alph, beta & gamma), Zeaxanthin, Gingo Biloba, Ginger, Cat'a Claw, Hypericum, Aloe Vera, Evening Primrose, Garlic, Capsicum, Dong Quai, Ginseng, Feverview, Fenugreek, Echinacea, Green Tea, Marshmallow, Saw Palmetto, Tea Tree Oil, Payllium, Kava-Kava, Licorice Root, Manonia Aquifolium, Hawthorne, Hohimbr, Tumeric, Witch Hazel, Valerian, Mistletoe, Bilberry, Bee Pollen, Peppermint Oil, Beta-Carotene, Genistein, Lutein, Lycopene, the Polyphenols (bioflavonoids), and the like.

After a start operation, the operational flow 200 includes a packaging operation 230 involving packaging the two or more nutraceutical agents in response to at least one of the one or more parameters associated with the individual. In some embodiments, one or more packaging units 114 may package the two or more nutraceutical agents 112 in response to at least one of the one or more parameters 106 associated with the individual 108.

Numerous types of packaging units 114 may be used to package two or more nutraceutical agents 112. In some embodiments, one packaging unit 114 is used to package two or more nutraceutical agents 112. In some embodiments, one or more packaging units 114 are used to package two or more nutraceutical agents 112. In some embodiments, two or more packaging units 114 are used to package two or more nutraceutical agents 112. In some embodiments, a first packaging unit 114 may package one or more first nutraceutical agents 112, a second packaging unit 114 may package one or more second nutraceutical agents 112, and a third packaging unit 114 may package the one or more first nutraceutical agents 112 and one or more second nutraceutical agents 112 together. In some embodiments, one packaging unit 114 may package the two or more nutraceutical agents 112. In some embodiments, one or more packaging units 114 may formulate two or more nutraceutical agents 112 for administration to an individual 108. In some embodiments, one or more packaging units 114 may package two or more preformulated nutraceutical agents 112 for administration to an individual 108. For example, in some embodiments, one or more packaging units 114 may package two or more commercially available nutraceutical preparations to provide for single administration to an individual 108. In some embodiments, one or more packaging units 114 may package two or more preformulated tablets containing the two or more nutraceutical agents 112 into a single capsule for administration to an individual 108. In some embodiments, one or more packaging units 114 may wrap a second nutraceutical agent 112 around a first nutraceutical agent 112 through use of a biocompatible and dissolvable wrapper to produce an administration form having the first and second nutraceutical agents 112 in concentric orientation relative to each other. In some embodiments, one or more packaging units 114 may package two or more nutraceutical agents 112 into a compartmentalized capsule. In some embodiments, one or more packaging units 114 may package two or more nutraceutical agents 112 into a single administration form for administration to an individual 108.

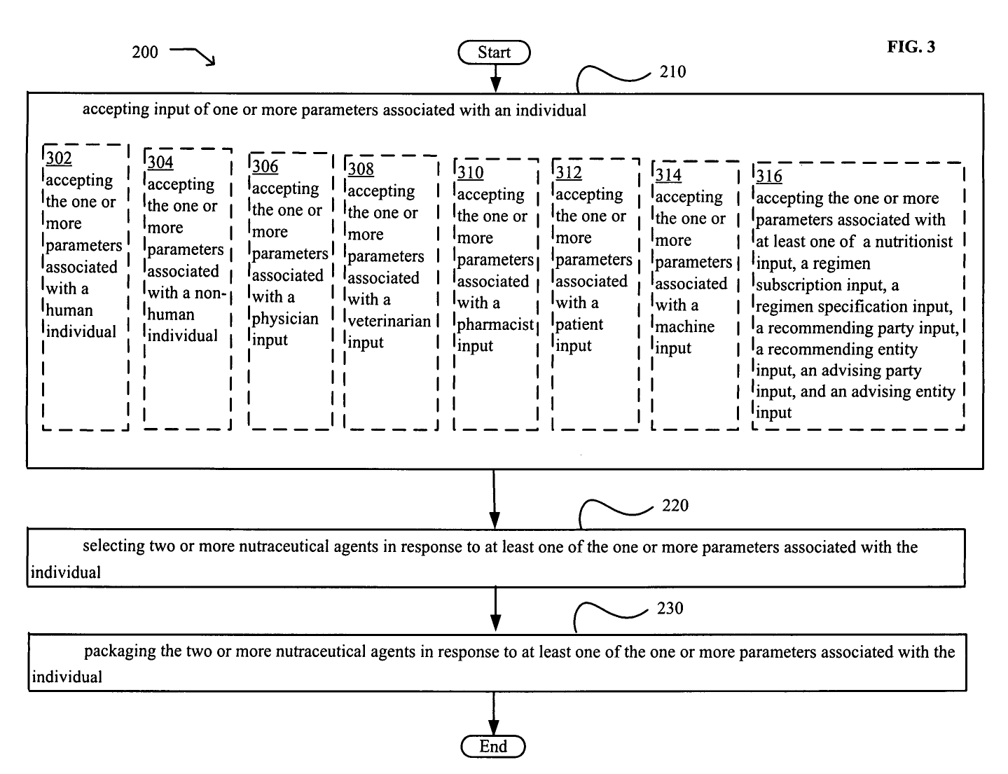

FIG. 3 illustrates alternative embodiments of the example operational flow 200 of FIG. 2. FIG. 3 illustrates example embodiments where the accepting operation 210 may include at least one additional operation. Additional operations may include an operation 302, operation 304, operation 306, operation 308, operation 310, operation 312, operation 314, and/or operation 316.

At operation 302, the accepting operation 210 may include accepting the one or more parameters associated with a human individual. In some embodiments, one or more accepting units 102 may accept the one or more parameters 106 associated with a human individual 108. In some embodiments, the one or more parameters 106 may include physical characteristics, metabolic characteristics, financial characteristics, and substantially any combination thereof. In some embodiments, such parameters 106 may include, alone or in combination and not limited to, an individual's height, weight, gender, kidney function, liver function, level of physical fitness, age, allergic response, metabolic level (i.e., resting metabolic rate and/or activity-related metabolic rate), disease state, body fat percentage, personal habits (i.e., smoking, alcohol consumption, diet, illegal drug use, and the like), family health history, insurance coverage, food supplement usage, physical activities, sleep schedule, activity level, occupation, nutraceutical usage, non-prescription drug use, prescription drug use, pregnancy status, predisposition toward the development of a malady, genotype, phenotype, genetic predisposition, administration form of a nutraceutical agent, mode of administration, time of administration, administration schedule, exposure to pathogens, potential exposure to pathogens, exposure to toxins, potential exposure to toxins, and the like. For example, in some embodiments, one or more parameters 106 associated with a human child may be input 104. Accordingly, such parameters 106 may provide for selection of one or more nutraceutical agents 112 that may be administered to a human child. In other embodiments, such parameters 106 may provide for selection against one or more nutraceutical agents 112 that should not be administered to a human child. Accordingly, in some embodiments, an input 104 may provide for the selection of one or more nutraceutical agents 112. However, in other embodiments, an input 104 may provide for selection against one or more nutraceutical agents 112. In some embodiments, parameters 106 may be input 104 that relate to environmental factors such as, time, temperature, elevation, humidity, events, activities and the like. For example, an input 104 may include parameters 106 related to an individual 108 who is a mountain climber. Accordingly, one or more nutraceutical agents 112 may be selected that will not vaporize under lessened atmospheric pressure, that will not freeze, and/or that will not break. In some embodiments, one or more parameters 106 may be input 104 that relate to administration form and mode of administration of the one or more nutraceutical agents 112 to the individual 108. For example, in some embodiments, one or more parameters 106 may be input 104 that indicate that the individual 108 prefers to orally ingest nutraceutical agents 112. In some embodiments, one or more parameters 106 may be input 104 that indicate that the individual 108 is to ingest two or more nutraceutical agents 112 within a given time period. Accordingly, in some embodiments, an input 104 may be associated with the selection of two or more nutraceutical agents 112 that are compatible with each other and/or that do not contraindicate each other. In some embodiments, an input 104 may be associated with the selection of two or more nutraceutical agents 112 that act in a synergistic manner when administered to an individual 108.

At operation 304, the accepting operation 210 may include accepting the one or more parameters associated with a non-human individual. In some embodiments, one or more accepting units 102 may accept the one or more parameters 106 associated with a non-human individual 108. Examples of such non-human animals include, but are not limited to, domestic pets such as dogs, cats, horses, potbelly pigs, ferrets, rodents, reptiles, amphibians, and the like. Non-human animals may also be animals that include, but are not limited to, cattle, sheep, goats, chickens, pigs, and the like. Accordingly, in some embodiments, the methods and/or systems described herein may be used for veterinary purposes. In some embodiments, the one or more parameters 106 may include physical characteristics, metabolic characteristics, financial characteristics (such as valuation of the non-human animal), and substantially any combination thereof. In some embodiments, such parameters 106 may include, alone or in combination and not limited to, a non-human individual's height, weight, gender, kidney function, liver function, level of physical fitness, age, allergic response, metabolic level (i.e., resting metabolic rate and/or activity-related metabolic rate), disease state, body fat percentage, health history, insurance coverage, food supplement usage, physical activities, sleep schedule, activity level, nutraceutical usage, non-prescription drug use, prescription drug use, pregnancy status, predisposition toward the development of a malady, genotype, phenotype, genetic predisposition, administration form, mode of administration, exposure to pathogens, potential exposure to pathogens, exposure to toxins, potential exposure to toxins, and the like. For example, in some embodiments, parameters 106 associated with an infant non-human individual 108 may be input 104. Accordingly, such parameters 106 may provide for selection of one or more nutraceutical agents 112 that may be administered to an infant non-human individual 108. In other embodiments, such parameters 106 may provide for selection against one or more nutraceutical agents 112 that should not be administered to an infant non-human individual 108. Accordingly, in some embodiments, an input 104 may provide for the selection of one or more nutraceutical agents 112. However, in other embodiments, an input 104 may provide for selection against one or more nutraceutical agents 112. In some embodiments, parameters 106 may be input 104 that relate to environmental factors surrounding the non-human individual 108 that include time, temperature, elevation, humidity, events, activities and the like. In some embodiments, one or more parameters 106 may be input 104 that relate to administration form and mode of administration of the one or more nutraceutical agents 112 to the non-human individual 108. For example, in some embodiments, one or more parameters 106 may be input 104 that indicate that one or more nutraceutical agents 112 should be administered to the non-human individual 108 orally. In some embodiments, one or more parameters 106 may be input 104 that indicate that the non-human individual 108 is to ingest two or more nutraceutical agents 112 within a given time period. Accordingly, in some embodiments, an input 104 may be associated with the selection of two or more nutraceutical agents 112 that are compatible with each other and/or that do not contraindicate each other. In some embodiments, an input 104 may be associated with the selection of two or more nutraceutical agents 112 that act in a synergistic manner when administered to a non-human individual 108.

At operation 306, the accepting operation 210 may include accepting the one or more parameters associated with a physician input. In some embodiments, one or more accepting units 102 may accept the one or more parameters 106 associated with a physician input 104. In some embodiments, one or more physicians may input 104 one or more parameters 106 associated with an individual 108. In some embodiments, one or more parameters 106 may be input 104 by one or more physicians and one or more other sources. Other sources of input 104 include, but are not limited to, veterinarian input 104, pharmacist input 104, patient input 104, machine input 104, nutritionist input 104, and the like. In some embodiments, one or more physicians may examine the individual 108 and input 104 one or more parameters 106 associated with the individual 108 that are related to the examination. For example, one or more physicians may input 104 one or more parameters 106 associated with an individual's heart rate, skin condition, allergy status, sleep status, and the like. In some embodiments, one or more physicians may input 104 one or more parameters 106 associated with an individual 108 without ever seeing the individual 108. For example, in some embodiments, one or more physicians may review a medical chart associated with the individual 108 and input 104 parameters 106 based on the information contained in the medical chart. In some embodiments, one or more physicians may input 104 parameters 106 associated with an individual 108 from the physician's memory. In some embodiments, one or more physicians may input 104 parameters 106 associated with an individual 108 following consultation with a database and/or other source of information. In some embodiments, one or more physicians may input 104 parameters 106 associated with an individual 108 directly through use of a keyboard, a touch-screen, and the like. In some embodiments, one or more physicians may input 104 parameters 106 associated with an individual 108 remotely through use of numerous technologies that include, input 104 from a wireless device, the internet, an intranet, a telephone, a palm held organizer, input 104 from a personal digital assistant, input 104 from a web enabled cellular telephone, and the like.

At operation 308, the accepting operation 210 may include accepting the one or more parameters associated with a veterinarian input. In some embodiments, one or more accepting units 102 may accept the one or more parameters 106 associated with a veterinarian input 104. In some embodiments, one or more veterinarians may input 104 one or more parameters 106 associated with a non-human individual 108. In some embodiments, one or more parameters 106 may be input 104 by one or more veterinarians and one or more other sources. Other sources of input 104 include, but are not limited to, physician input 104, pharmacist input 104, patient input 104, machine input 104, nutritionist input 104, and the like. In some embodiments, one or more veterinarians may examine a non-human individual 108 and input 104 one or more parameters 106 associated with the non-human individual 108 that are related to the examination. For example, one or more veterinarians may input 104 one or more parameters 106 associated with a non-human individual's heart rate, skin condition, allergy status, sleep status, and the like. In some embodiments, one or more veterinarians may input 104 one or more parameters 106 associated with a non-human individual 108 without ever seeing the non-human individual 108. For example, in some embodiments, one or more veterinarians may review a medical chart associated with the non-human individual 108 and input 104 parameters 106 based on the information contained in the medical chart. In some embodiments, one or more veterinarians may input 104 parameters 106 associated with a non-human individual 108 from the veterinarian's memory. In some embodiments, one or more veterinarians may input 104 parameters 106 associated with a non-human individual 108 following consultation with a database and/or other source of information. In some embodiments, one or more veterinarians may input 104 parameters 106 associated with a non-human individual 108 directly through use of a keyboard, a touch-screen, and the like. In some embodiments, one or more veterinarians may input 104 parameters 106 associated with a non-human individual 108 remotely through use of numerous technologies that include, input 104 from a wireless device, the internet, an intranet, a telephone, a palm held organizer, input 104 from a personal digital assistant, input 104 from a web enabled cellular telephone, and the like.

At operation 310, the accepting operation 210 may include accepting the one or more parameters associated with a pharmacist input. In some embodiments, one or more accepting units 102 may accept the one or more parameters 106 associated with a pharmacist input 104. In some embodiments, one or more pharmacists may input 104 one or more parameters 106 associated with an individual 108. In some embodiments, one or more parameters 106 may be input 104 by one or more pharmacists and one or more other sources. Other sources of input 104 include, but are not limited to, physician input 104, veterinarian input 104, patient input 104, machine input 104, nutritionist input 104, and the like. In some embodiments, one or more pharmacists may consult with an individual 108 and input 104 one or more parameters 106 associated with the individual 108 that are related to the consultation. For example, one or more pharmacists may input 104 one or more parameters 106 associated with an individual's heart rate, skin condition, allergy status, sleep status, and the like. In some embodiments, one or more pharmacists may input 104 one or more parameters 106 associated with an individual 108 without ever seeing the individual 108. For example, in some embodiments, one or more pharmacists may receive information associated with the individual 108 and input 104 parameters 106 based on the received information. In some embodiments, one or more pharmacists may input 104 parameters 106 associated with an individual 108 from the pharmacist's memory. In some embodiments, one or more pharmacists may input 104 parameters 106 associated with an individual 108 following consultation with a database and/or other source of information. In some embodiments, one or more pharmacists may input 104 parameters 106 associated with an individual 108 directly through use of a keyboard, a touch-screen, and the like. In some embodiments, one or more pharmacists may input 104 parameters 106 associated with an individual 108 remotely through use of numerous technologies that include, input 104 from a wireless device, the internet, an intranet, a telephone, a palm held organizer, input 104 from a personal digital assistant, input 104 from a web enabled cellular telephone, and the like.

At operation 312, the accepting operation 210 may include accepting the one or more parameters associated with a patient input. In some embodiments, one or more accepting units 102 may accept the one or more parameters 106 associated with a patient input 104. In some embodiments, a patient may input 104 one or more parameters 106 associated with the patient. In some embodiments, one or more parameters 106 may be input 104 by the patient and one or more other sources. Other sources of input 104 include, but are not limited to, physician input 104, pharmacist input 104, patient input 104, machine input 104, nutritionist input 104, and the like. In some embodiments, a patient may input 104 one or more parameters 106 associated with the patient's heart rate, skin condition, allergy status, sleep status, and the like. In some embodiments, a patient may input 104 parameters 106 associated with the patient following consultation with a database and/or other source of information. In some embodiments, a patient may input 104 parameters 106 associated with the patient directly through use of a keyboard, a touch-screen, and the like. In some embodiments, a patient may input 104 parameters 106 associated with the patient remotely through use of numerous technologies that include, input 104 from a wireless device, the internet, an intranet, a telephone, a palm held organizer, input 104 from a personal digital assistant, input 104 from a web enabled cellular telephone, and the like. In some embodiments, a patient may input 104 parameters 106 associated with nutraceutical agents 112 that are being administered to the patient. In some embodiments, a patient may input 104 parameters 106 associated with one or more times of administration of one or more nutraceutical agents 112.

At operation 314, the accepting operation 210 may include accepting the one or more parameters associated with a machine input. In some embodiments, one or more accepting units 102 may accept the one or more parameters 106 associated with a machine input 104. In some embodiments, the one or more parameters 106 may include physical characteristics, metabolic characteristics, financial characteristics, and substantially any combination thereof. In some embodiments, such parameters 106 may include, alone or in combination and not limited to, an individual's height, weight, gender, kidney function, liver function, level of physical fitness, age, allergic response, metabolic level (i.e., resting metabolic rate and/or activity-related metabolic rate), disease state, body fat percentage, personal habits (i.e., smoking, alcohol consumption, diet, illegal drug use, and the like), family health history, insurance coverage, food supplement usage, physical activities, sleep schedule, activity level, occupation, nutraceutical usage, non-prescription drug use, prescription drug use, pregnancy status, predisposition toward the development of a malady, genotype, phenotype, genetic predisposition, administration form of a nutraceutical agent, mode of administration, time of administration, administration schedule, exposure to pathogens, potential exposure to pathogens, exposure to toxins, potential exposure to toxins, and the like. For example, in some embodiments, one or more parameters 106 associated with a human child may be input 104. Accordingly, such parameters 106 may provide for selection of one or more nutraceutical agents 112 that may be administered to a human child. In other embodiments, such parameters 106 may provide for selection against one or more nutraceutical agents 112 that should not be administered to a human child. Accordingly, in some embodiments, an input 104 may provide for the selection of one or more nutraceutical agents 112. However, in other embodiments, an input 104 may provide for selection against one or more nutraceutical agents 112. In some embodiments, parameters 106 may be input 104 that relate to environmental factors such as, time, temperature, elevation, humidity, events, activities and the like. For example, an input 104 may include parameters 106 related to an individual 108 who is a mountain climber. Accordingly, one or more nutraceutical agents 112 may be selected that will not vaporize under lessened atmospheric pressure, that will not freeze, and/or that will not break. In some embodiments, one or more parameters 106 may be input 104 that relate to administration form and mode of administration of the one or more nutraceutical agents 112 to the individual 108. For example, in some embodiments, one or more parameters 106 may be input 104 that indicate that the individual 108 prefers to orally ingest nutraceutical agents 112. In some embodiments, one or more parameters 106 may be input 104 that indicate that the individual 108 is to ingest two or more nutraceutical agents 112 within a given time period. Accordingly, in some embodiments, an input 104 may be associated with the selection of two or more nutraceutical agents 112 that are compatible with each other and/or that do not contraindicate each other. In some embodiments, an input 104 may be associated with the selection of two or more nutraceutical agents 112 that act in a synergistic manner when administered to an individual 108. In some embodiments, the machine is a diagnostic machine that has been utilized during examination of the individual 108.

At operation 316, the accepting operation 210 may include accepting the one or more parameters associated with at least one of a nutritionist input, a regimen subscription input, a regimen specification input, a recommending party input, a recommending entity input, an advising party input, or an advising entity input. In some embodiments, one or more accepting units 102 may accept the one or more parameters associated with at least one of a nutritionist input 104, a regimen subscription input 104, a regimen specification input 104, a recommending party input 104, a recommending entity input 104, an advising party input 104, and an advising entity input 104. In some embodiments, input 104 may include one or more parameters 106 associated with an individual 108. In some embodiments, input 104 may include one or more parameters associated with an individual 108 that are input 104 by one or more sources. Other sources of input 104 include, but are not limited to, physician input 104, veterinarian input 104, patient input 104, machine input 104, pharmacist input 104, regimen subscription input 104, regimen specification input 104, recommending party input 104, recommending entity input 104, advising party input 104, advising entity input 104, and the like. In some embodiments, one or more sources of input 104 may consult with an individual 108 and input 104 one or more parameters 106 associated with the individual 108 that are related to the consultation. For example, one or more nutritionists may input 104 one or more parameters 106 associated with an individual's heart rate, skin condition, allergy status, sleep status, and the like. In some embodiments, one or more nutritionists may input 104 one or more parameters 106 associated with an individual 108 without ever seeing the individual 108. For example, in some embodiments, one or more nutritionists may receive information associated with the individual 108 and input 104 parameters 106 based on the received information. In some embodiments, one or more nutritionists may input 104 parameters 106 associated with an individual 108 from the nutritionist's memory. In some embodiments, one or more nutritionists may input 104 parameters 106 associated with an individual 108 following consultation with a database and/or other source of information. In some embodiments, one or more nutritionists may input 104 parameters 106 associated with an individual 108 directly through use of a keyboard, a touch-screen, and the like. In some embodiments, one or more nutritionists may input 104 parameters 106 associated with an individual 108 remotely through use of numerous technologies that include, input 104 from a wireless device, the internet, an intranet, a telephone, a palm held organizer, input 104 from a personal digital assistant, input 104 from a web enabled cellular telephone, and the like. Input 104 may be associated with grocery stores, food supplement stores, personal trainers, coaches, clinics, hospitals, dental offices, veterinary offices, and the like.

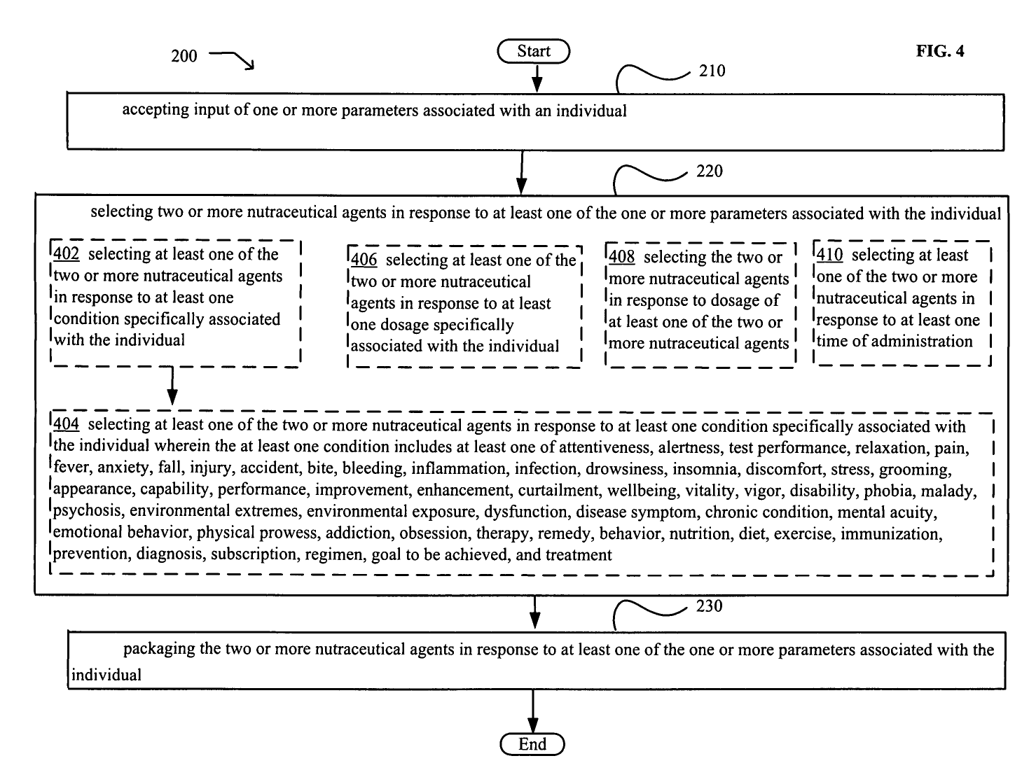

FIG. 4 illustrates alternative embodiments of the example operational flow 200 of FIG. 2. FIG. 4 illustrates example embodiments where the selecting operation 220 may include at least one additional operation. Additional operations may include an operation 402, operation 404, operation 406, operation 408 and/or operation 410.

At operation 402, the selecting operation 220 may include selecting at least one of the two or more nutraceutical agents in response to at least one condition specifically associated with the individual. In some embodiments, one or more selecting units 110 may select at least one of the two or more nutraceutical agents 112 in response to at least one condition specifically associated with the individual 108.

In some embodiments, a condition specifically associated with an individual 108 may be an existing condition. In some embodiments, an existing condition is a medical condition. Examples of such medical conditions include, but are not limited to, viral infection, bacterial infection, fungal infection, diabetes, arthritis, gastrointestinal maladies, cancer, allergic responses, psychological disorders, osteoporosis, Alzheimer's disease, asthma, chronic fatigue syndrome, epilepsy, heart disease, hemochromatosis, hepatitis, stroke, food intolerance, and the like in substantially any combination. Accordingly, one or more nutraceutical agents 112 may be selected to reduce or ameliorate the symptoms of a condition and/or to treat the condition directly. Numerous nutraceutical agents 112 that may be selected in response to a condition are known (i.e., Roberts et al., Nutraceuticals: The Complete Encyclopedia of Supplements, Herbs, Vitamins, and Healing Foods, 1^{st} Edition, Perigee Trade (2001); Rapport and Lockwood, Nutraceuticals, Pharmaceutical Press (2001); Wildman, Handbook of Nutraceuticals and Functional Foods, CRC Press, 1^{st} Edition, (2000); Eskin, Dictionary of Nutraceuticals and Functional Foods (Functional Foods and Nutraceuticals), CRC Press, (2005); The PDR Family Guide to Nutritional Supplements: An Authoritative A-to-Z Resource on the 100 Most Popular Nutritional Therapies and Nutraceuticals; Ballantine Books, 1^{st} Edition, (2001); Susan G. Wynn, Emerging Therapies: Using Herbs and Nutraceuticals for Small Animals, American Animal Hospital Assn Press (1999); U.S. Pat. Nos. 7,045,159; 7,049,433; 7,041,840; 6,979,679; 6,962,720; 6,881,425; The Merck Index, 13^{th} Edition, An Encyclopedia of Chemicals, Drugs, and Biologicals, Merck & Co. Inc., Whitehouse Station, N.J. (2001); Mosby's Drug Guide, Mosby, Inc., St. Louis, Mo. (2004); Remington: The Science and Practice of Pharmacy, 20^{th} Edition, Lippincott Williams & Wilkins, Philadelphia, Pa. (2000); Physicians' Desk Reference, 58^{th} Edition, Thompson, PDR, Montvale, N.J. (2004); U.S. Pat. No. 6,773,721; herein incorporated by reference).

In some embodiments, a condition specifically associated with an individual 108 may be a past condition. For example, one or more nutraceutical agents 112 may be selected such that a condition, such as a medical condition, that an individual 108 was treated for in the past will be disallowed from reoccurring or the condition, or symptoms of the condition, may be reduced or minimized if the condition were to reoccur in the individual 108. For example, in some embodiments, one or more nutraceutical agents 112 may be selected to prevent or reduce the consequences of a heart attack that may reoccur in an individual 108. In some embodiments, one or more nutraceutical agents 112 may be selected to prevent or reduce the consequences of an epileptic seizure in an individual 108. Accordingly, one or more nutraceutical agents 112 may be selected in response to numerous past conditions associated with the individual 108.

In some embodiments, a condition specifically associated with an individual 108 may be a future condition. For example, one or more nutraceutical agents 112 may be selected such that a condition, such as a medical condition, that an individual 108 is predisposed to developing in the future may be disallowed from occurring or the condition, or symptoms of the condition, may be reduced or minimized if the condition were to occur in the individual 108. Accordingly, one or more nutraceutical agents 112 may be selected in response to numerous future conditions associated with the individual 108. In some embodiments, one or more nutraceutical agents 112 may be selected to prevent the occurrence of a future condition. In some embodiments, one or more nutraceutical agents 112 may be selected in response to conditions that are cyclic. For example, in some embodiments, one or more nutraceutical agents 112 may be selected in response to a woman's menstrual cycle. In other embodiments, one or more nutraceutical agents 112 may be selected in response to a psychological malady, such as depression, that occurs in a cyclic manner. In other embodiments, one or more nutraceutical agents 112 may be selected in response to hormonal changes that are expected to occur in the future, such as menopause.

In some embodiments, a condition specifically associated with an individual 108 may be an event or activity associated with an individual 108. For example, in some embodiments, one or more nutraceutical agents 112 may be selected in response to a condition that is an event associated with an individual 108. For example, in some embodiments, an individual 108 may be expecting to participate in a sporting event. Accordingly, one or more nutraceutical agents 112 may be selected in response to the event such that the one or more agents will not interfere with the performance of the individual 108. In other examples, the one or more nutraceutical agents 112 may be selected to improve performance of the individual 108 in the event. In some embodiments, an individual 108 may expect to give a presentation. Accordingly, one or more nutraceutical agents 112 may be selected that will not interfere with the performance of the individual 108 or that will improve performance of the individual 108 giving the presentation.

In some embodiments, a condition specifically associated with an individual 108 may be related to the environment in which the individual 108 resides or expects to reside. For example, if an individual 108 expects to travel on a boat, one or more nutraceutical agents 112 may be selected that will not contribute to, or that will reduce or ameliorate, motion sickness. In some embodiments, the one or more nutraceutical agents 112 may be selected based on the climactic environment in which an individual 108 resides or expects to reside. For example, one or more nutraceutical agents 112 may be selected based on temperature, humidity, atmospheric pressure, and the like in substantially any combination. In some embodiments, the one or more nutraceutical agents 112 may be selected based on the biological environment in which an individual 108 resides or expects to reside. For example, one or more nutraceutical agents 112 may be selected based on the presence of allergens, pathogens, infectious agents, toxins, organisms and the like in substantially any combination.

In some embodiments, a condition specifically associated with an individual 108 may be a condition known to be associated with the individual 108 or a condition thought to be associated with an individual 108. For example, in some embodiments, one or more nutraceutical agents 112 may be selected that can be used to treat an individual 108 with a diagnosed condition. In other embodiments, one or more nutraceutical agents 112 may be selected that can be administered to an individual 108 with an undiagnosed condition with which the individual 108 was believed to be affected in the in the past, present or future. For example, in some embodiments, 5-hydroxytryptophan, s-adenosylmethionine, St. John's wort, Kava kava, Ginko biloba, melatonin, and/or substantially any combination thereof may be a selected for administration to an individual 108 to reduce or eliminate symptoms associated with depression. In other embodiments, glucosamine and/or chondroitan may be selected for administration to an individual 108 to rebuild cartilage that cushions and protects joints. In some embodiments, small quantitites of lithium may be used to reduce or eliminate symptoms associated with manic/depressive (bipolar) and depressive disorders associated with an individual 108. In other embodiments, small amounts of lithium or choline in combination with vitamin supplements may be used to reduce or eliminate symptoms associated with manic/depressive (bipolar) and depressive disorders associated with an individual 108.

At operation 404, the selecting operation 220 may include selecting at least one of the two or more nutraceutical agents in response to at least one condition specifically associated with the individual wherein the at least one condition includes at least one of attentiveness, alertness, test performance, relaxation, pain, fever, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, goal to be achieved, or treatment. In some embodiments, one or more selecting units 110 may include selecting at least one of the two or more nutraceutical agents 112 in response to at least one condition specifically associated with the individual wherein the at least one condition includes at least one of attentiveness, alertness, test performance, relaxation, pain, fever, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, goal to be achieved, and treatment. Accordingly, in some embodiments, at least one of the two or more nutraceutical agents may be selected in response to an existing condition. In some embodiments, at least one of the two or more nutraceutical agents may be selected in reponse to a goal that is to be achieved in the future. Examples of such goals include, but are not limited to, attentiveness, alertness, increased test performance, relaxation, and the like.

At operation 406, the selecting operation 220 may include selecting at least one of the two or more nutraceutical agents in response to at least one dosage specifically associated with the individual. In some embodiments, one or more selecting units 110 may select at least one of the two or more nutraceutical agents 112 in response to at least one dosage specifically associated with the individual 108.

In some embodiments, one or more selecting units 110 may select one or more nutraceutical agents 112 with regard to a volume of one or more of the nutraceutical agents 112. For example, one or more selecting units 110 may select a first nutraceutical agent 112 preferentially over a second nutraceutical agent 112 if the first nutraceutical agent 112 occupies less volume than the second nutraceutical agent 112. In other examples, one or more selecting units 110 may select a first nutraceutical agent 112 preferentially over a second nutraceutical agent 112 if the first nutraceutical agent 112 occupies more volume than the second nutraceutical agent 112. Accordingly, one or more nutraceutical agents 112 may be selected to increase or decrease the volume of the administration form of the one or more nutraceutical agents 112 to promote administration to an individual 108.

In some embodiments, one or more selecting units 110 may select one or more nutraceutical agents 112 with regard to the compatibility of the nutraceutical agents 112 with each other or with the individual 108 at the dosage associated with the individual 108. For example, in some embodiments, one or more nutraceutical agents 112 may be selected that are compatible with each other in response to dosage of at least one of the nutraceutical agents 112. In some embodiments, one or more nutraceutical agents 112 may be selected to act synergistically with each other when administered to an individual 108 at a given dosage. In some embodiments, one or more nutraceutical agents 112 may be selected to avoid synergistic interactions with each other when administered to an individual 108 at a given dosage. In some embodiments, one or more nutraceutical agents 112 may be selected to counteract or reduce any negative side-effects of the one or more nutraceutical agents 112 when they are administered to an individual 108 at a given dosage. In some embodiments, one or more nutraceutical agents 112 may be selected with regard to dosage so that they do not contraindicate additional components, such as pharmaceuticals and/or food supplements, ingested by an individual 108. In some embodiments, one or more nutraceutical agents 112 may be selected with regard to the price of the one or more nutraceutical agents 112 with regard to one or more dosages associated with an individual 108. For example, in some embodiments, a nutraceutical agent 112 may be commercially available at two or more dosages that are priced differently. Accordingly, in some embodiments, the one or more nutraceutical agents 112 may be selected to achieve a desired dosage when administered to an individual 108 while reducing or minimizing the price associated with the one or more nutraceutical agents 112.

At operation 408, the selecting operation 220 may include selecting the two or more nutraceutical agents in response to dosage of at least one of the two or more nutraceutical agents. In some embodiments, one or more selecting units 110 may select the two or more nutraceutical agents 112 in response to dosage of at least one of the two or more nutraceutical agents 112.

In some embodiments, one or more nutraceutical agents 112 may be commercially available in preformulated administration forms. Accordingly, in some embodiments, one or more nutraceutical agents 112 may be selected in response to administration forms that are commercially available. For example, in some embodiments, a nutraceutical agent 112 may be commercially available in 100 milligram, 250 milligram, 500 milligram, 750 milligram, and 1000 milligram preformulated administration forms. In some instances, an individual 108 may be prescribed or instructed to ingest 750 milligram of a nutraceutical agent 112. Accordingly, in some embodiments, a 750 milligram administration form of the nutraceutical agent 112 may be selected. In other embodiments, a 250 milligram and a 500 milligram administration form of the nutraceutical agent 112 may be selected. In other embodiments, a 250 milligram and five 100 milligram administration forms of the nutraceutical agent 112 may be selected. Numerous combinations of administration forms may be selected. In some embodiments, administration forms may be selected with regard to price associated with the administration form. For example, in some embodiments, it may be less expensive to achieve a 750 milligram dosage of a nutraceutical agent 112 by combining one 250 milligram administration form with five 100 milligram administration forms than selecting a single 750 milligram administration form.

In some embodiments, one or more nutraceutical agents 112 may be selected with regard to administration forms for administration to an individual 108 over one or more periods of time. For example, it may be desirable to administer 1000 milligrams of a nutraceutical agent 112 to an individual 108 over a ten hour time period. Accordingly, in some embodiments, a single 1000 milligram controlled release administration form may be selected. In other embodiments, ten 100 milligram administration forms may be selected and then packaged to be released at a rate of one 100 milligram administration form per hour over the ten hour period. Accordingly, numerous combinations of administration forms and timed release may be selected.

In some embodiments, one or more selecting units 110 may select one or more nutraceutical agents 112 with regard to one or more volumes of one or more of the nutraceutical agents 112 in the available administration forms. For example, one or more selecting units 110 may select a first nutraceutical agent 112 preferentially over a second nutraceutical agent 112 if the first nutraceutical agent 112 occupies less volume than the second nutraceutical agent 112 with regard to available administration forms. In other examples, one or more selecting units 110 may select a first nutraceutical agent 112 preferentially over a second nutraceutical agent 112 if the first nutraceutical agent 112 occupies more volume than the second nutraceutical agent 112 with regard to available administration forms. Accordingly, one or more nutraceutical agents 112 may be selected to increase or decrease the volume of the one or more nutraceutical agents 112 to promote administration to an individual 108.

In some embodiments, one or more selecting units 110 may select one or more nutraceutical agents 112 with regard to compatibility of the nutraceutical agents 112 with each other and/or with the individual 108 when administered to the individual 108 at dosages corresponding to available administration forms of the nutraceutical agents 112. For example, in some embodiments, one or more nutraceutical agents 112 may be selected in response to administration forms available for the two or more nutraceutical agents 112. In some embodiments, two or more nutraceutical agents 112 may be selected to act synergistically with each other when administered to an individual 108 at available administration forms. In some embodiments, one or more nutraceutical agents 112 may be selected to avoid synergistic interactions with each other when administered to an individual 108 as available administration forms. In some embodiments, one or more nutraceutical agents 112 may be selected to counteract or reduce any negative side-effects of the one or more nutraceutical agents 112 when they are administered to an individual 108 at an available dosage. In some embodiments, one or more nutraceutical agents 112 may be selected with regard to available dosage so that they do not contraindicate additional components, such as pharmaceuticals and/or food supplements, ingested by an individual 108. In some embodiments, one or more nutraceutical agents 112 may be selected with regard to the price of the one or more nutraceutical agents 112 with regard to one or more available dosages associated with the one or more nutraceutical agents 112. For example, in some embodiments, a nutraceutical agent 112 may be commercially available at two or more dosages that are priced differently. Accordingly, in some embodiments, the one or more nutraceutical agents 112 may be selected to achieve a desired dosage when administered to an individual 108 while reducing or minimizing the price associated with the one or more nutraceutical agents 112.

At operation 410, the selecting operation 220 may include selecting at least one of the two or more nutraceutical agents in response to at least one time of administration. In some embodiments, one or more selecting units 110 may select at least one of the two or more nutraceutical agents 112 in response to at least one time of administration

In some embodiments, the at least one time of administration is a time when the one or more nutraceutical agents 112 are to be administered to an individual 108 to provide for release of the one or more nutraceutical agents 112 from the administration form at a specified time following administration. For example, in some embodiments, at least one of the two or more nutraceutical agents 112 may be selected such that it is released from an administration form about one hour after being administered to an individual 108. In other embodiments, a first nutraceutical agent 112 may be selected such that it is released from an administration form about one hour after being administered to an individual 108 and a second nutraceutical agent 112 may be selected such that it is released from an administration form about two hours after being administered to the individual 108. Accordingly, one or more nutraceutical agents 112 may be selected that are released from an administration form at a specified time following administration to an individual 108 and thereupon become functionally available to the individual 108. In some embodiments, two or more incompatible nutraceutical agents 112 may be administered to an individual 108 at the same time without adverse consequences by providing for release of the incompatible nutraceutical agents 112 at different times such that they do not contraindicate each other. In some embodiments, two or more nutraceutical agents 112 that act synergistically may be coadministered to an individual 108 such that they are released at substantially the same time to provide for synergistic action of the two or more nutraceutical agents 112 with regard to the individual 108. Substantially any combination of nutraceutical agents 112, dosages and release times may be selected.

In some embodiments, the at least one time of administration is relative to a time or event preceding or following administration of one or more nutraceutical agents 112 to an individual 108. Accordingly, one or more nutraceutical agents 112 may be selected that are released from an administration form at a relative time following administration to an individual 108 and thereupon become functionally available to the individual 108. For example, in some embodiments, two or more nutraceutical agents 112 may be coadministered to an individual 108 such that a first nutraceutical agent 112 is released from the administration form and a second nutraceutical agent 112 is released from the administration form at a second time that is relative to the time of release of the first nutraceutical agent 112. Accordingly, in some embodiments, two or more incompatible nutraceutical agents 112 may be administered to an individual 108 at the same time without adverse consequences by providing for release of the incompatible nutraceutical agents 112 at different times such that they do not contraindicate each other. In some embodiments, two or more nutraceutical agents 112 that act synergistically may be coadministered to an individual 108 such that they are released at substantially the same time to provide for synergistic action of the two or more nutraceutical agents 112 with regard to the individual 108. In some embodiments, dosages of the two or more nutraceutical agents 112 may be altered in a relative manner. For example, in some embodiments, the dosage of two or more nutraceutical agents 112 may be calibrated relative to time of day. In other embodiments, the dosage of two or more nutraceutical agents 112 may be calibrated relative to hormonal cycles. In other embodiments, the dosage of two or more nutraceutical agents 112 may be calibrated relative to circadian rhythms. Substantially any combination of nutraceutical agents 112, dosages and release times may be selected relative to a time, event and/or the like.

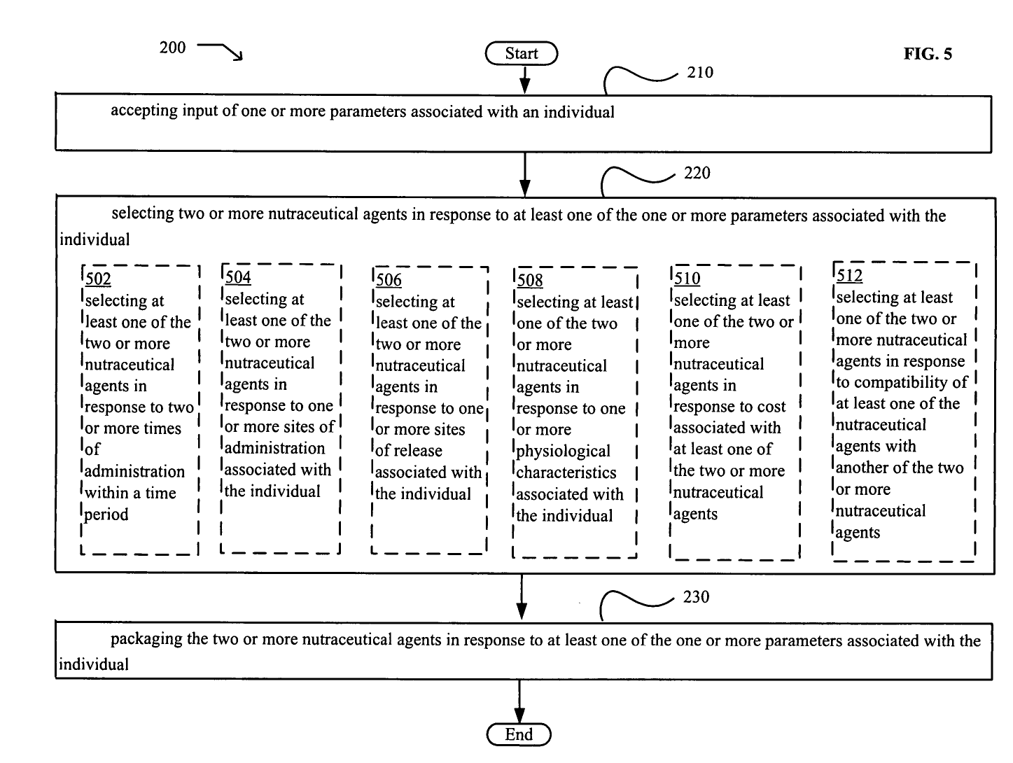

FIG. 5 illustrates alternative embodiments of the example operational flow 200 of FIG. 2. FIG. 5 illustrates example embodiments where the selecting operation 220 may include at least one additional operation. Additional operations may include an operation 502, operation 504, operation 506, operation 508, operation 510, and/or operation 512.

At operation 502, the selecting operation 220 may include selecting at least one of the two or more nutraceutical agents in response to two or more times of administration within a time period. In some embodiments, one or more selecting units 110 may select at least one of the two or more nutraceutical agents 112 in response to two or more times of administration within a time period. In some embodiments, a time period is defined as being a discrete amount of time. For example, in some embodiments, a time period may be defined in seconds, minutes, hours, days, months, years and substantially any combination thereof. In some embodiments, a time period may be defined as being an amount of time that is relative to a measurable quantity and/or event. For example, in some embodiments, a time period may be determined based on the concentration of a nutraceutical agent 112 that was previously administered to an individual 108. Accordingly, in some embodiments, a first nutraceutical agent 112 may be administered to an individual 108 and a second nutraceutical agent 112 may be administered to the same individual 108 when the concentration of the first nutraceutical agent 112 associated with the individual 108 either reaches a certain level or decreases to a certain level. Numerous combinations of discrete and/or relative amounts of time may be used during the selection of at least one of two or more nutraceutical agents 112. In some embodiments, at least one of the two or more nutraceutical agents 112 may be selected based on the identity of a second nutraceutical agent 112 that is to be administered to an individual 108 within a time period in which the first nutraceutical agent 112 is still present and/or functionally active in association with an individual 108. For example, in some embodiments, a first nutraceutical agent 112 is selected such that it does not contraindicate a second nutraceutical agent 112 that is to be administered to the individual 108 within a time period when the first and second nutraceutical agents 112 are both present and/or functionally active in association with the individual 108. In some embodiments, the second nutraceutical agent 112 is selected such that it does not contraindicate a first nutraceutical agent 112 that is present and/or functionally active in association with the individual 108. In some embodiments, a first nutraceutical agent 112 is selected such that it will act in a synergistic manner with a second nutraceutical agent 112 that is to be administered to the individual 108 within a time period when the first and second nutraceutical agents 112 are both present and/or functionally active in association with the individual 108. In some embodiments, the second nutraceutical agent 112 is selected such that it will act in a synergistic manner with a first nutraceutical agent 112 that is present and/or functionally active in association with the individual 108.

At operation 504, the selecting operation 220 may include selecting at least one of the two or more nutraceutical agents in response to one or more sites of administration associated with the individual. In some embodiments, one or more selecting units 110 may select at least one of the two or more nutraceutical agents 112 in response to one or more sites of administration associated with the individual 108. One or more nutraceutical agents 112 may be administered at numerous sites associated with an individual 108. Examples of such sites include, but are not limited to, the eyes, ears, nose, skin, mouth, stomach, intestine, rectum, vagina, vascular system, pulmonary system, gastrointestinal system, urinary system and lymphatic system. In some embodiments, one or more nutraceutical agents 112 may be administered at a first site associated with an individual 108 in preference to a second site associated with an individual 108. For example, in some embodiments, it may be desirable to administer a nutraceutical agent 112 that is acid labile by injection into the vascular system in preference to oral administration which may expose the nutraceutical agent 112 to acidic conditions. Accordingly, in some embodiments, one or more nutraceutical agents 112 may be selected based on the physical and chemical characteristics of the one or more nutraceutical agents 112 and where the one or more nutraceutical agents 112 will be administered to an individual 108. In some embodiments, one or more nutraceutical agents 112 may be selected in response to the site of action of the one or more nutraceutical agents 112 on an individual 108. For example, in some embodiments, an adhesive patch may be used to administer one or more nutraceutical agents 112 for the treatment of a malady associated with the skin. In some embodiments, one or more first nutraceutical agents 112 may be selected for administration to a first site associated with an individual 108 and one or more second nutraceutical agents 112 may be selected such that the second nutraceutical agents 112 facilitate administration of the first nutraceutical agents 112, do not contraindicate the first nutraceutical agents 112, act synergistically with the first nutraceutical agents 112, are administered to a second site associated with the individual 108, and/or substantially any combination thereof.

At operation 506, the selecting operation 220 may include selecting at least one of the two or more nutraceutical agents in response to one or more sites of release associated with the individual. In some embodiments, one or more selecting units 110 may select at least one of the two or more nutraceutical agents 112 in response to one or more sites of release associated with the individual 108. In some embodiments, one or more nutraceutical agents 112 may be administered to an individual 108 at a first site and then released from the administration form in which the nutraceutical agents 112 were administered at a second site associated with the individual 108. For example, in some embodiments, one or more nutraceutical agents 112 may be administered to an individual 108 in an oral administration form which can be released in the small intestine of the individual 108. In examples of other embodiments, one or more nutraceutical agents 112 may be released into the vascular system of an individual 108 following transdermal administration of the one or more nutraceutical agents 112 to the individual 108. In some embodiments, two or more nutraceutical agents 112 may be coadministered to an individual 108 such that they are released from their administration forms at two or more separate sites associated with the individual 108. For example, in some embodiments, a first and second nutraceutical agent 112 may be coadministered to an individual 108 such that the first nutraceutical agent 112 is substantially released from the administration form in the upper gastrointestinal tract and the second nutraceutical agent 112 is substantially released from the administration form in the lower gastrointestinal tract. Accordingly, in some embodiments, two or more nutraceutical agents 112 that are incompatible or that would contraindicate each may be coadministered to an individual 108 for release at different sites associated with the individual 108 and/or at different times.

At operation 508, the selecting operation 220 may include selecting at least one of the two or more nutraceutical agents in response to one or more physiological characteristics associated with the individual. In some embodiments, one or more selecting units 110 may select at least one of the two or more nutraceutical agents 112 in response to one or more physiological characteristics associated with the individual 108. Numerous physiological characteristics may be associated with an individual 108. Examples of such characteristics include, but are not limited to, age, gender, disease state, allergic responses, activity-related metabolic rate, resting metabolic rate, liver function, kidney function, weight, body fat percentage, epithelial cell function, lung function, skin function, gastrointestinal tract function, and substantially any combination thereof. Methods to predict drug response and to assess and correlate metabolism to drug dosage are known (i.e., International Publication Numbers: WO 03/084395 and WO 2005/041105; U.S. Pat. Nos. 6,317,719 and 6,087,090, herein incorporated by reference). Such methods may also be used to predict and to assess and correlate metabolism of a nutraceutical agent 112 by an individual 108. Accordingly, numerous assays may be used to assess the ability of an individual 108 to metabolize one or more nutraceutical agents 112. In some embodiments, enzyme activities may be assessed to determine the ability of an individual 108 to metabolize one or more nutraceutical agents 112. Examples of such enzyme systems and activities that may be assessed include, but are not limited to, the cytochrome P450 monooxygenase system, the flavin-containing monooxygenase system, alcohol dehydrogenase, aldehyde dehydrogenase, monoamine oxidase, cooxidation by peroxidases, NADPH-cytochrome P450 reductase, the presence of reduced (ferrous) cytochrome P450, esterases, amidases, epoxide hydrolase, glutathione S-transferases, mercapturic acid biosynthesis, UDP-glucoron(os)yltransferases, N-Acetyltransferases, amino acid N-acyl transferases and sulfotransferases. In some embodiments, first and second nutraceutical agents 112 may be effective to treat the same condition associated with an individual 108. However, an individual 108 may be able to metabolize the first nutraceutical agent 112 very quickly but metabolize a second nutraceutical agent 112 more slowly. Accordingly, in some embodiments, the second nutraceutical agent 112 may be selected for administration to the individual 108 to avoid higher relative metabolism of the first nutraceutical agent 112 by the individual 108. In some embodiments, an individual 108 may mount an adverse allergic response to one or more nutraceutical agents 112. Accordingly, one or more nutraceutical agents 112 may be selected to avoid or minimize allergic response to administration of the one or more nutraceutical agents 112 to the individual 108. One or more nutraceutical agents 112, and combinations of one or more nutraceutical agents 112, may be selected in response to numerous physiological characteristics associated with an individual 108.

At operation 510, the selecting operation 220 may include selecting at least one of the two or more nutraceutical agents in response to cost associated with at least one of the two or more nutraceutical agents. In some embodiments, one or more selecting units 110 may select at least one of the two or more nutraceutical agents 112 in response to cost associated with at least one of the two or more nutraceutical agents 112. In some embodiments, two or more different nutraceutical agents 112 may be used to treat the same or a similar condition associated with an individual 108. In some embodiments, it may preferable to select a first nutraceutical agent 112 having a lower associated cost over a second nutraceutical agent 112 having a higher associated cost for administration to an individual 108. In other embodiments, it may be preferable to select a first nutraceutical agent 112 having a higher associated cost over a second nutraceutical agent 112 having a lower associated cost for administration to an individual 108. In some embodiments, one or more nutraceutical agents 112 may be selected in response to cost associated with the one or more nutraceutical agents 112 and numerous additional considerations. Such additional considerations include, but are not limited to, allergic response, dosage, effectiveness, interaction with other nutraceutical agents 112 and substantially any combination thereof.

At operation 512, the selecting operation 220 may include selecting at least one of the two or more nutraceutical agents in response to compatibility of at least one of the nutraceutical agents with another of the two or more nutraceutical agents. In some embodiments, one or more selecting units 110 may select at least one of the two or more nutraceutical agents 112 in response to compatibility of at least one of the nutraceutical agents 112 with another of the two or more nutraceutical agents 112. In some embodiments, at least one of the nutraceutical agents 112 is selected that does not interact with another of the two or more nutraceutical agents 112. In some embodiments, at least one of the nutraceutical agents 112 is selected to act in a synergistic manner with another of the two or more nutraceutical agents 112. In some embodiments, at least one of the nutraceutical agents 112 is selected to not contraindicate at least one of the two or more nutraceutical agents 112.

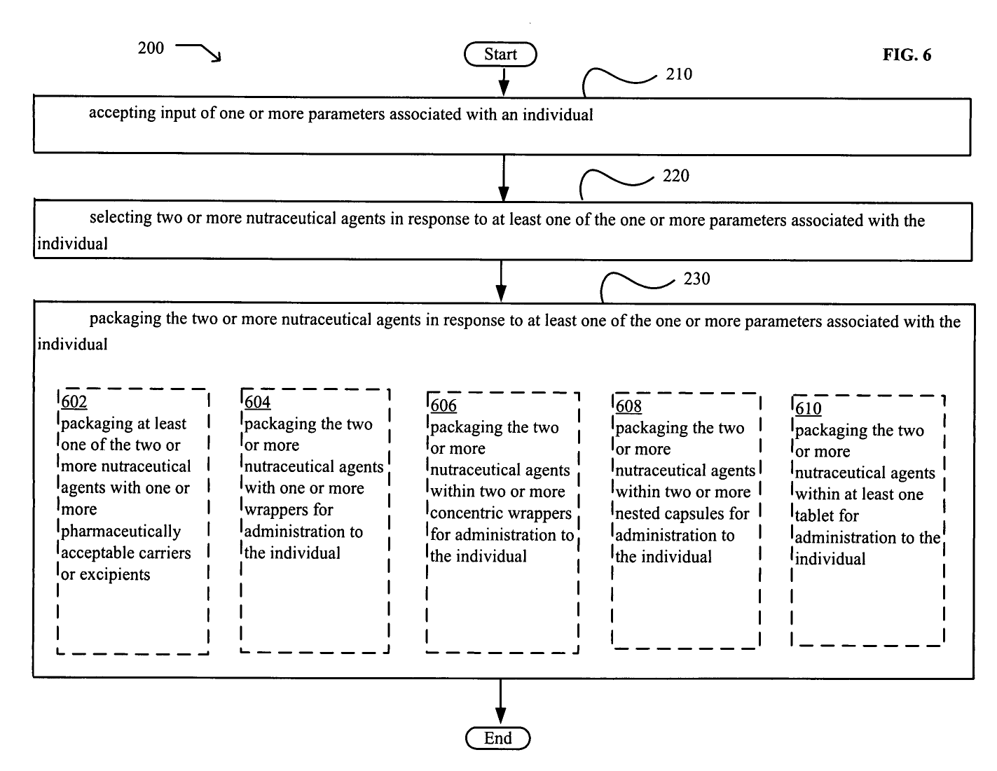

FIG. 6 illustrates alternative embodiments of the example operational flow 200 of FIG. 2. FIG. 6 illustrates example embodiments where the packaging operation 230 may include at least one additional operation. Additional operations may include an operation 602, operation 604, operation 606, operation 608 and/or operation 610.

At operation 602, the packaging operation 230 may include packaging at least one of the two or more nutraceutical agents with one or more pharmaceutically acceptable carriers or excipients. In some embodiments, one or more packaging units 114 may package at least one of the two or more nutraceutical agents 112 with one or more pharmaceutically acceptable carriers or excipients.

Nutraceutical agents 112 may be packaged through use of numerous known methods, such as conventional mixing, dissolving, granulating, dragee-making, levigating, emulsifying, encapsulating, entrapping or lyophilizing processes. In some embodiments, the nutraceutical agents 112 may be packaged in a manner that depends on the route that the nutraceutical agents 112 are to be administered to an individual 108.

In some embodiments, one or more nutraceutical agents 112 may be packaged with one or more solid or gel phase carriers or excipients. Examples of such carriers or excipients include, but are not limited to, croscarmellose sodium, povidone, microcrystalline cellulose, calcium carbonate, calcium phosphate, various sugars, starches, cellulose derivatives, gelatin, pregelatinized starch, polymers such as polyethylene glycols, lactose, lactose monohydrate, sucrose, talc, gelatin, agar, pectin, acacia, magnesium stearate, stearic acid and substantially any combination thereof. If a solid carrier is used, the one or more nutraceutical agents 112 may be tableted, placed in a hard gelatin capsule in powder or pellet form, packaged in the form of a troche or lozenge, and the like.

In some embodiments, one or more nutraceutical agents 112 may be packaged with a liquid carrier or excipient. Examples of such liquid carriers include syrup, peanut oil, olive oil, water, physiologically compatible buffers (i.e., Hanks solution and Ringers solution), physiological saline buffer, and the like. If a liquid carrier is used, the administration form may be in the form of a syrup, emulsion, drop, soft gelatin capsule, sterile injectable solution, suspension in an ampoule or vial, non-aqueous liquid suspension, and the like.

One or more nutraceutical agents 112 may be packaged in stable water-soluble administration forms. For example, in some embodiments, a pharmaceutically acceptable salt of one or more nutraceutical agents 112 may be dissolved in an aqueous solution of an organic or inorganic acid, such as 0.3M solution of succinic acid or citric acid. If a soluble salt form is not available, a nutraceutical agent 112 may be dissolved in a suitable cosolvent or combination of cosolvents. Examples of suitable cosolvents include, but are not limited to, alcohol, propylene glycol, polyethylene glycol 300, polysorbate 80, glycerin and the like in concentrations ranging from 0-60% of the total volume. In some embodiments, one or more nutraceutical agents 112 may be dissolved in DMSO and diluted with water. The administration form may also be in the form of a solution of a salt form of one or more nutraceutical agents 112 in an appropriate aqueous vehicle such as water or isotonic saline or dextrose solution.

In some embodiments, nutraceutical agents 112 that are hydrophobic may be packaged through use of a cosolvent system comprising benzyl alcohol, a nonpolar surfactant, a water-miscible organic polymer, and an aqueous phase. The cosolvent system may be the VPD co-solvent system. VPD is a solution of 3 percent weight/volume benzyl alcohol, 8 percent weight/volume of the nonpolar surfactant polysorbate 80, and 65 percent weight/volumen polyethylene glycol 300, made up to volume in absolute ethanol. The VPD co-solvent system (VPD:5W) consists of VPD diluted 1:1 with a 5 percent dextrose in water solution. This co-solvent system dissolves hydrophobic pharmaceutical agents well, and itself produces low toxicity upon systemic administration. Accordingly, the co-solvent system may also be used to dissolve hydrophobic nutraceutical agents 112. The proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol (i.e., polyvinyl pyrrolidone; and other sugars or polysaccharides may substitute for dextrose). Many other delivery systems may be used to administer hydrophobic nutraceutical agents 112 as well. For example, liposomes and emulsions are well known examples of delivery vehicles or carriers for hydrophobic drugs that may also be used to deliver nutraceuticals. Certain organic solvents such as dimethysulfoxide also may be employed, although usually at the cost of greater toxicity.

Some nutraceutical agents 112 may be packaged as salts with compatible counter ions. Compatible salts may be formed with many acids, including hydrochloric, sulfuric, acetic, lactic, tartaric, malic, succinic, etc. Salts of nutraceutical agents 112 may be more soluble in aqueous or other protonic solvents than are the corresponding free-base forms.

Numerous carriers and excipients are known and are commercially available (i.e., The Merck Index, 13^{th} Edition, An Encyclopedia of Chemicals, Drugs, and Biologicals, Merck & Co. Inc., Whitehouse Station, N.J. 2001; Mosby's Drug Guide, Mosby, Inc., St. Louis, Mo. 2004; Remington: The Science and Practice of Pharmacy, 20^{th} Edition, Lippincott Williams & Wilkins, Philadelphia, Pa. 2000; Physicians' Desk Reference, 58^{th} Edition, Thompson, PDR, Montvale, N.J. 2004; U.S. Pat. Nos. 6,773,721; 7,053,107; 7,049,312 and Published U.S. Patent Application No. 20040224916; herein incorporated by reference).

At operation 604, the packaging operation 230 may include packaging the two or more nutraceutical agents with one or more wrappers in response to at least one of the one or more parameters associated with the individual. In some embodiments, one or more packaging units 114 may package the two or more nutraceutical agents 112 with one or more wrappers in response to at least one of the one or more parameters 106 associated with the individual 108. In some embodiments, two or more nutraceutical agents 112 may be packaged by wrapping the two or more nutraceutical agents 112 into a single administration form for administration to an individual 108. In some embodiments, the two or more nutraceutical agents 112 may be preformulated prior to being wrapped in one or more wrappers. For example, two or more nutraceutical agents 112 that are each in tablet form may be wrapped into a single administration form. In other embodiments, the two or more nutraceutical agents 112 may be combined together and then wrapped in one or more wrappers. In other embodiments, two or more nutraceutical agents 112 may be combined together with a suitable carrier and then wrapped in one or more wrappers. Numerous materials may be used to wrap the two or more nutraceutical agents 112. Examples of such materials include, but are not limited to, polymers that include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methamethacrylate copolymers, shellac, and the like. Numerous water insoluble polymers may be used that include cellulose derivatives (i.e., ethylcellulose), polyvinyl acetate, neutral copolymers based on ethyl acrylate and methylmethacrylate, copolymers of acrylic and methacrylic acid esters with quaternary ammonium groups, and the like. In some embodiments, polymers used in forming the wrappers may be plasticized. Examples of plasticizers that may be used to plasticize the wrappers include, but are not limited to, triacetin, tributyl citrate, triethyl citrate, acetyl tri-n-butyl citrate diethyl phthalate, castor oil, dibutyl sebacate, acetylated monoglycerides, and the like and/or substantially any combination thereof. In some embodiments, the plasticizer may be present at about 3 to 30 weight percent and more typically about 10 to 25 weight percent based on the polymer to which the plasticizer is added. The type of plasticizer and its content depends on the polymer or polymers, nature of the coating system. In some embodiments, water-soluble nonionic polysaccharide derivatives may be used to wrap one or more nutraceutical agents 112. For example, hydroxypropylmethylcellulose, hydroxypropylcellulose, and/or sodium carboxymethylcellulose may be used. Such polymers form coatings that quickly dissolve in water and have a high permeability. Accordingly, in some embodiments, such polymers may be used for rapid release of one or more nutraceutical agents 112 that are wrapped in such a wrapper following administration to an individual 108. In some embodiments, one or more nutraceutical agents 112 may be wrapped in a wrapper that provides for sustained release of the one or more nutraceutical agents 112. For example, one or more nutraceutical agents 112 may be released continuously over twelve hours through use of wrappers constructed from ethyl cellulose and an ethyl acrylate-methyl methacrylate-ethyl trimethylammoniumchloride methacrylate copolymer as the release controlling wrapper. Methods and materials that may be used to prepare wrappers are known in the art and are commercially available (i.e., Rohm Pharma, Piscataway, N.J.; U.S. Pat. Nos. 6,656,507; 7,048,945; 7,056,951; hereby incorporated by reference).

In some embodiments, one wrapper may be used to wrap two or more nutraceutical agents 112 into an administration form. For example, the two or more nutraceutical agents 112 may be combined together and then wrapped into an administration form in one wrapper for release at the same time following administration to an individual 108. In other embodiments, one continuous wrapper may be used to wrap the two or more nutraceutical agents 112 into an administration form in which the two or more nutraceutical agents 112 are separated from each other. For example, in some embodiments, one of the two or more nutraceutical agents 112 may be covered with a continuous wrapper to form a core and then a second nutraceutical agent 112 may be wrapped around the core with the continuous wrapper to form an administration form. This process may be repeated with multiple nutraceutical agents 112 to form a multilayered administration form in which the multiple nutraceutical agents 112 are separated from each other. In some embodiments, such a configuration provides for the release of nutraceutical agents 112 from the administration form at different times and/or at different sites associated with an individual 108 to which the administration form is administered. In some embodiments, two or more nutraceutical agents 112 are wrapped into an administration form together and additional nutraceutical agents 112 are wrapped into the administration form in separate layers. Accordingly, nutraceutical agents 112 may be oriented in the administration form to be released from the administration form at the same time and/or site or such that they are released at different times and/or sites. Examples of such sites include, but are not limited to, the mouth, esophagus, stomach, duodenum, small intestine, large intestine, and the rectum.

At operation 606, the packaging operation 230 may include packaging the two or more nutraceutical agents within two or more concentric wrappers in response to at least one of the one or more parameters associated with the individual. In some embodiments, one or more packaging units 114 may package the two or more nutraceutical agents 112 within two or more concentric wrappers in response to at least one of the one or more parameters 106 associated with the individual 108. In some embodiments, two or more nutraceutical agents 112 may be packaged by wrapping the two or more nutraceutical agents 112 within two or more wrappers to form an administration form. In some embodiments, the same type of material is used to form the two or more wrappers in the administration form. In some embodiments, different types of material are used as wrappers to form the administration form. For example, an outer wrapper may be selected to dissolve rapidly and release one or more nutraceutical agents 112 soon after administration of the administration form to the individual 108 while an inner wrapper may be selected to release one or more nutraceutical agents 112 at a later time and/or at a different site associated with an individual 108. Accordingly, in some embodiments, multiple nutraceutical agents 112 may be packaged into the same administration form for release at different times and at different sites following administration of the administration form to an individual 108. In some embodiments, the nutraceutical agents 112 may be the same to provide for continuous dosing of an individual 108. In some embodiments, the nutraceutical agents 112 may be different to provide for dosing of an individual 108 with different nutraceutical agents 112. In some embodiments, some of the nutraceutical agents 112 may be the same to provide for continuous dosing of an individual 108 and others may be different to provide for dosing of an individual 108 with different nutraceutical agents 112. Accordingly, numerous combinations of nutraceutical agents 112 and wrappers may be assembled into an administration form.

At operation 608, the packaging operation 230 may include packaging the two or more nutraceutical agents within two or more nested capsules in response toat least one of the one or more parameters associated with the individual. In some embodiments, one or more packaging units 114 may package the two or more nutraceutical agents 112 within two or more nested capsules in response to at least one of the one or more parameters associated with the individual 108.

In some embodiments, two or more nutraceutical agents 112 may be packaged into an administration form through use of nested capsules. In some embodiments, a first nutraceutical agent 112 may be packaged in a first capsule and a second nutraceutical agent 112 may be packaged in a second capsule in which the first capsule is included to create an administration form having nested capsules. Accordingly, administration forms may be constructed that include two or more nested capsules. In some embodiments, such administration forms may include two or more nutraceutical agents 112. In other embodiments, such administration forms may include one type of nutraceutical agent 112 that is contained within multiple capsules of the administration form and one or more types of different nutraceutical agents 112 that are also contained within the capsules included within the administration form. In some embodiments, the material used to construct the individual capsules of a single administration form is the same. In some embodiments, the material used to construct the individual capsules of a single administration form is different. In some embodiments, the material used to construct some of the individual capsules of a single administration form may be the same while the material used to construct other individual capsules of the single administration form may be different. Accordingly, through selection of materials used to construct the individual capsules contained in an administration form, two or more nutraceutical agents 112 may be released from one administration form at one or more times and/or at one or more sites associated with the individual 108. For example, as with wrapping materials described herein, materials may be selected for constructing capsules that release one or more nutraceutical agents 112 at a site associated with an individual 108. Examples of such sites include, but are not limited to, the mouth, esophagus, stomach, duodenum, small intestine, large intestine, and the rectum.

At operation 610, the packaging operation 230 may include packaging the two or more nutraceutical agents within at least one tablet in response toat least one of the one or more parameters associated with the individual. In some embodiments, one or more packaging units 114 may package the two or more nutraceutical agents 112 within at least one tablet in response to at least one of the one or more parameters associated with the individual 108. In some embodiments, two or more nutraceutical agents 112 may be selected in response to one or more parameters 106 associated with an individual 108 and packaged into at least one table. Accordingly, in some embodiments, two or more nutraceutical agents 112 may be packaged into a tablet such that the two or more nutraceutical agents 112 are released at the same or different times following administration of the tablet to an individual 108. In other embodiments, two or more nutraceutical agents 112 may be packaged into a tablet such that the two or more nutraceutical agents 112 are released at the same or different sites associated with an individual 108 following administration of the tablet to an individual 108. In other embodiments, two or more nutraceutical agents 112 may be packaged into a tablet such that the two or more nutraceutical agents 112 are released at the same or different times and at the same or different sites associated with an individual 108 following administration of the tablet to the individual 108.

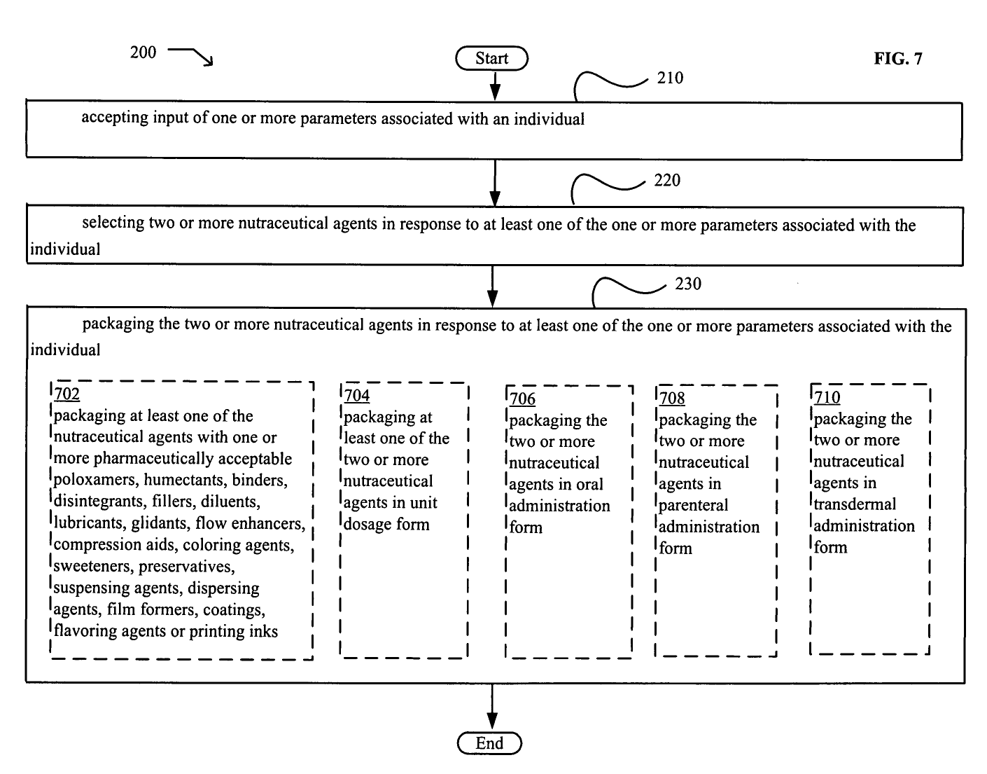

FIG. 7 illustrates alternative embodiments of the example operational flow 200 of FIG. 2. FIG. 7 illustrates example embodiments where the packaging operation 230 may include at least one additional operation. Additional operations may include an operation 702, operation 704, operation 706, operation 708 and/or operation 710.

At operation 702, the packaging operation 230 may include packaging at least one of the nutraceutical agents with one or more pharmaceutically acceptable poloxamers, humectants, binders, disintegrants, fillers, diluents, lubricants, glidants, flow enhancers, compression aids, coloring agents, sweeteners, preservatives, suspensing agents, dispersing agents, film formers, coatings, flavoring agents or printing inks. In some embodiments, one or more packaging units 114 may package at least one of the nutraceutical agents 112 with one or more pharmaceutically acceptable poloxamers, humectants, binders, disintegrants, fillers, diluents, lubricants, glidants, flow enhancers, compression aids, coloring agents, sweeteners, preservatives, suspending agents, dispersing agents, film formers, coatings, flavoring agents or printing inks.

At operation 704, the packaging operation 230 may include packaging at least one of the two or more nutraceutical agents in unit dosage form. In some embodiments, one or more packaging units 114 may package at least one of the two or more nutraceutical agents 112 in unit dosage form. The term “unit dosage form” refers to one or more amounts of one or more nutraceutical agents 112 that are suitable as unitary dosages for individuals, such as human and non-human individuals, with each unit containing a predetermined quantity of at least one nutraceutical agent 112 calculated to produce a desired effect, such as a therapeutic effect, in association with one or more suitable pharmaceutical carriers. Such unit dosage forms may be packaged in numerous configurations that include, but are not limited to, tablets, capsules, ampoules, and other administration forms known in the art and described herein. In some embodiments, two or more unit dosage forms of one or more nutraceutical agents 112 may be packaged into an administration form. For example, in some embodiments, two unit dosage forms may be wrapped into an administration form through use of a continuous wrapper such that they are released at different times following administration to an individual 108. In such an example, two unit dosage forms are included within one administration form. Accordingly, numerous combinations of nutraceutical agents 112 and unit dosage forms may be included within an administration form.

At operation 706, the packaging operation 230 may include packaging the two or more nutraceutical agents in oral administration form. In some embodiments, one or more packaging units 114 may package the two or more nutraceutical agents 112 in oral administration form.

For oral administration, one or more nutraceutical agents 112 may be packaged into an oral administration form by combining the one or more nutraceutical agents 112 with pharmaceutically acceptable carriers that are well known in the art. Such carriers allow the one or more nutraceutical agents 112 to be formulated as tablets, pills, dragees, capsules, liquids, gels, syrups, slurries, suspensions and the like, for oral ingestion by an individual 108. Oral administration forms can be obtained by combining the one or more nutraceutical agents 112 with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of nutraceutical agents 112.

Oral administration forms may include push-fit capsules made of gelatin, as well as soft, sealed capsules made of gelatin and a plasticizer, such as glycerol or sorbitol. The push-fit capsules can contain one or more nutraceutical agents 112 in admixture with a filler such as lactose, binders such as starches, and/or lubricants such as talc or magnesium stearate and, optionally, stabilizers. In soft capsules, the nutraceutical agents 112 may be dissolved or suspended in suitable liquids, such as fatty oils, liquid paraffin, or liquid polyethylene glycols. In addition, stabilizers may be added. All oral dosage forms may be prepared in dosages suitable for such administration. For buccal administration, the nutraceutical agents 112 may take the form of tablets or lozenges formulated in a conventional manner.

At operation 708, the packaging operation 230 may include packaging the two or more nutraceutical agents in parenteral administration form. In some embodiments, one or more packaging units 114 may package the two or more nutraceutical agents 112 in parenteral administration form.

The one or more nutraceutical agents 112 may be formulated for parenteral administration by injection (i.e., bolus injection or continuous infusion). Formulations for injection may be presented in unit dosage form (i.e., in ampoules or in multi-dose containers) with an added preservative. The administration forms may take such forms as suspensions, solutions or emulsions in oily or aqueous vehicles, and may contain formulatory agents such as suspending, stabilizing and/or dispersing agents.

Administration forms for parenteral administration may include aqueous solutions of the one or more nutraceutical agents 112 in water-soluble form. In some embodiments, the one or more nutraceutical agents 112 may be formulated in physiologically compatible buffers that include Hanks solution, Ringers solution, physiological saline buffer, and the like. Additionally, suspensions of the one or more nutraceutical agents 112 may be prepared as appropriate oily injection suspensions. Suitable lipophilic solvents include fatty oils such as sesame oil, or synthetic fatty acid esters, such as ethyl oleate or triglycerides, or liposomes. Aqueous injection suspensions may include substances which increase the viscosity of the suspension, such as sodium carboxymethyl cellulose, sorbitol, or dextran. Optionally, the suspension may also contain suitable stabilizers or agents which increase the solubility of the one or more nutraceutical agents 112 to allow for the preparation of highly concentrated solutions.

At operation 710, the packaging operation 230 may include packaging the two or more nutraceutical agents in transdermal administration form. In some embodiments, one or more packaging units 114 may package the two or more nutraceutical agents 112 in transdermal administration form. For transdermal, including transmucosal, administration of the one or more nutraceutical agents 112, penetrants appropriate to the barrier or barriers to be permeated may be used in the formulation. Briefly, in some embodiments, a transdermal administration form may include an ethoxylated lipid, an alcohol mixed with the ethoxylated lipid to form a penetration enhancer, an aqueous adjuvant mixed with the penetration enhancer, and a delivered nutraceutical agent 112 mixed with the aqueous adjuvant and the penetration enhancer. In some embodiments, the aqueous adjuvant is a plant extract from the family of Liliaceae Liliaceae. In some embodiments, the ethoxylated lipid is a vegetable oil or animal oil having at least 20 ethoxylations per molecule. In other embodiments, about 0.1 percent to 40.0 percent by weight or volume is ethoxylated lipid. Other embodiments may include a transdermal delivery system that includes about 0.1 percent to 15 percent by weight or volume of alcohol or where about 0.1 percent to 85 percent by weight or volume is Aloe Vera. Numerous transdermal administration forms are known and have been described (i.e., U.S. Pat. Nos. 5,820,876; 7,045,145; 6,946,144; incorporated herein by reference).

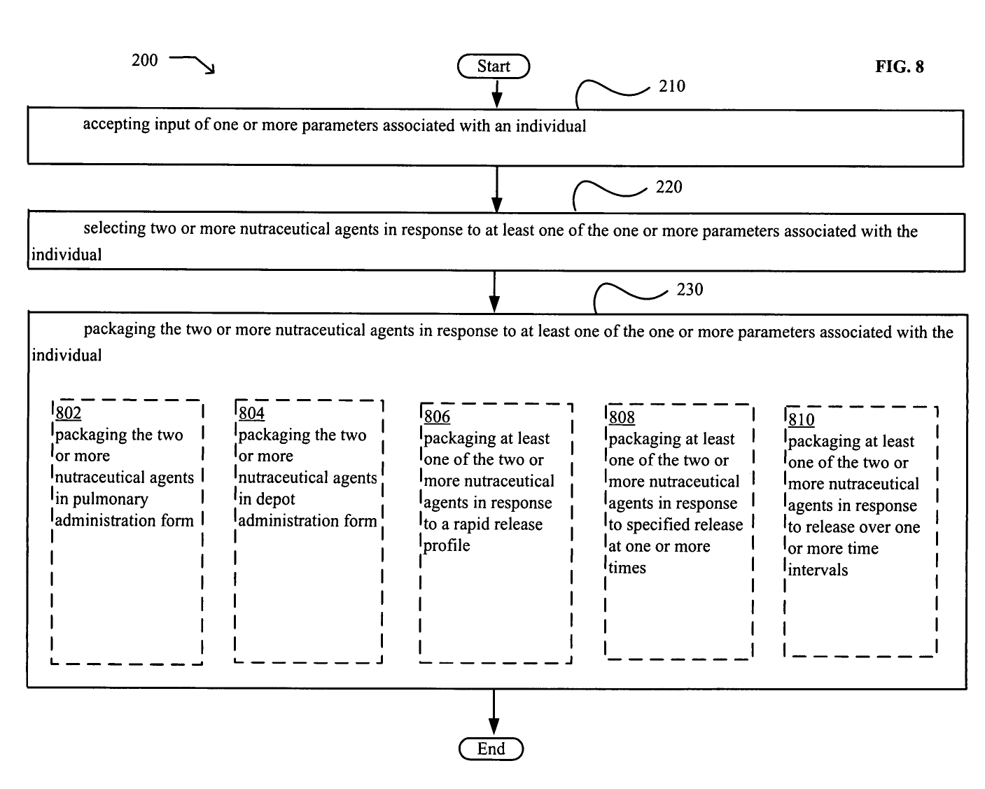

FIG. 8 illustrates alternative embodiments of the example operational flow 200 of FIG. 2. FIG. 8 illustrates example embodiments where the packaging operation 230 may include at least one additional operation. Additional operations may include an operation 802, operation 804, operation 806, operation 808 and/or operation 810.

At operation 802, the packaging operation 230 may include packaging the two or more nutraceutical agents in pulmonary administration form. In some embodiments, one or more packaging units 114 may package the two or more nutraceutical agents 112 in pulmonary administration form. For pulmonary administration, the one or more nutraceutical agents 112 may be delivered in the form of an aerosol spray from pressurized packs or a nebuliser, with the use of a suitable propellant (i.e., dichlorodifluoromethane, trichlorofluoromethane, dichlorotetrafluoroethane, carbon dioxide or other suitable gas). In the case of a pressurized aerosol, the dosage unit may be determined by providing a valve to deliver a metered amount of the one or more nutraceutical agents 112. Capsules and cartridges for use in an inhaler or insufflator may be formulated to contain a powder mix of the one or more nutraceutical agents 112 and a suitable powder base such as lactose or starch. Methods and materials that may be used to package one or more nutraceutical agents 112 in pulmonary administration form are known and have been described (i.e., U.S. Pat. Nos. 6,921,527; 6,838,076; 6,565,841; 6,451,286; 6,169,068; 5,993,783; 5,780,014; 5,719,123; 5,354,934; 5,284,656; 5,006,343; hereby incorporated by reference).

At operation 804, the packaging operation 230 may include packaging the two or more nutraceutical agents in depot administration form. In some embodiments, one or more packaging units 114 may package the two or more nutraceutical agents 112 in depot administration form. In some embodiments, depot administration forms may be administered by implantation (i.e., subcutaneously, intramuscularly, intramuscular injection, subtenon, intravitreal injection). Accordingly, for example, the one or more nutraceutical agents 112 may be packaged with suitable polymeric or hydrophobic materials, ion exchange resins, and the like. Methods and materials that may be used to package nutraceutical agents 112 in depot administration form are known and are commercially available (i.e., U.S. Pat. Nos. 6,773,714; 6,630,155; 6,565,874; 5,945,115; herein incorporated by reference).

At operation 806, the packaging operation 230 may include packaging at least one of the two or more nutraceutical agents in response to a rapid release profile. In some embodiments, one or more packaging units 114 may package at least one of the two or more nutraceutical agents 112 in response to a rapid release profile. In some embodiments, water-soluble nonionic polysaccharide derivatives may be used to package one or more nutraceutical agents 112. For example, hydroxypropylmethylcellulose, hydroxypropylcellulose, and/or sodium carboxymethylcellulose may be used. Such polymers form coatings that quickly dissolve in water and have a high permeability. Accordingly, in some embodiments, such polymers may be used for rapid release of one or more nutraceutical agents 112 that are packaged in such materials following administration to an individual 108. Numerous rapid release formulations are known and have been described (i.e., U.S. Pat. No. 6,979,463; herein incorporated by reference).

At operation 808, the packaging operation 230 may include packaging at least one of the two or more nutraceutical agents in response to specified release at one or more times. In some embodiments, one or more packaging units 114 may package at least one of the two or more nutraceutical agents 112 in response to specified release at one or more times. In some embodiments, one or more nutraceutical agents 112 may be packaged so that they are released from an administration form at one or more times following administration to an individual 108. In some embodiments, one or more nutraceutical agents 112 may be released at one or more times following administration to maintain the dosage of the one or more nutraceutical agents 112 at or above a certain concentration. Accordingly, in some embodiments, the concentration of one nutraceutical agent 112 may be maintained over a period of time in association with an individual 108. In other embodiments, the concentration of more than one nutraceutical agent 112 may be maintained over a period of time in association with an individual 108. In some embodiments, one or more nutraceutical agents 112 may be packaged to be released in anticipation of an event, such as a long airplane flight. For example, in some embodiments, one or more nutraceutical agents 112 that induce sleep may be packaged into an administration form so that an individual 108 to whom the administration form is administered will fall asleep at a precalculated time on an airplane during a long flight. In other embodiments, one or more nutraceuticals may be packaged into an administration form such that an individual 108 to whom the administration form is administered will not fall asleep during a long meeting or presentation. Numerous methods may be used to package one or more nutraceutical agents 112 for release at one or more times. For example, in some embodiments, one or more nutraceutical agents 112 may be wrapped into an administration form through methods described herein. In such examples, the time of release of the one or more nutraceutical agents 112 from the administration form may be controlled through selection of wrappers used to formulate the administration form. For example, a thick wrapper may be used to delay release while a thin wrapper may be used to expedite release of the one or more nutraceutical agents 112 from the administration form. In other embodiments, one or more wrappers may be selected that are made of material that is more or less resistant to degradation when administered to an individual 108. Accordingly, materials having various chemical and physical properties may be selected to produce administration forms that release one or more nutraceutical agents 112 at one or more times.

At operation 810, the packaging operation 230 may include packaging at least one of the two or more nutraceutical agents in response to release over one or more time intervals. In some embodiments, one or more packaging units 114 may package at least one of the two or more nutraceutical agents 112 in response to release over one or more time intervals. In some embodiments, one or more nutraceutical agents 112 may be packaged so that they are released from an administration form over one or more time intervals following administration to an individual 108. In some embodiments, one or more nutraceutical agents 112 may be released over one or more times following administration to maintain the dosage of the one or more nutraceutical agents 112 at or above a certain concentration. Accordingly, in some embodiments, the concentration of one nutraceutical agent 112 may be maintained over a period of time in association with an individual 108. In other embodiments, the concentration of more than one nutraceutical agent 112 may be maintained over a period of time in association with an individual 108. In some embodiments, one or more nutraceutical agents 112 may be packaged to be released over one or more time intervals in anticipation of an event, such as a long airplane flight, that may occur during the one or more time intervals. For example, in some embodiments, one or more nutraceutical agents 112 that induce sleep may be packaged into an administration form so that they are released during the time interval in which an individual 108 to whom the administration form is administered is on an airplane. Numerous methods may be used to package one or more nutraceutical agents 112 for release over one or more time intervals. For example, in some embodiments, one or more nutraceutical agents 112 may be wrapped into an administration form through methods described herein. In such examples, the time of release of the one or more nutraceutical agents 112 from the administration form may be controlled through selection of wrappers used to prepare the administration form. For example, a thick wrapper may be used to delay release while a thin wrapper may be used to expedite release of the one or more nutraceutical agents 112 from the administration form. In other embodiments, one or more wrappers may be selected that are made of material that is more or less resistant to degradation when administered to an individual 108. In other embodiments, controlled-release formulations may be acquired and then packaged for release over one or more time intervals.

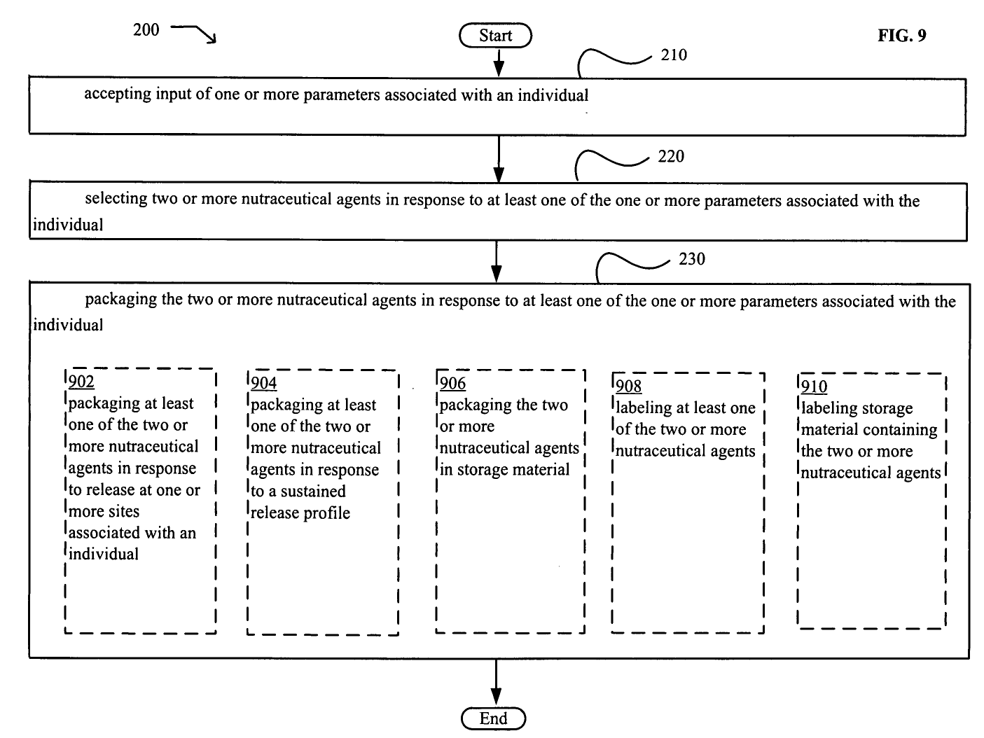

FIG. 9 illustrates alternative embodiments of the example operational flow 200 of FIG. 2. FIG. 9 illustrates example embodiments where the packaging operation 230 may include at least one additional operation. Additional operations may include an operation 902, operation 904, operation 906, operation 908 and/or operation 910.

At operation 902, the packaging operation 230 may include packaging at least one of the two or more nutraceutical agents in response to release at one or more sites associated with an individual. In some embodiments, one or more packaging units 114 may package at least one of the two or more nutraceutical agents 112 in response to release at one or more sites associated with an individual 108. One or more nutraceutical agents 112 may be packaged for administration to numerous sites that are associated with an individual 108. Examples of such sites include, but are not limited to, the eyes, ears, nose, skin, mouth, stomach, intestine, rectum, vagina, vascular system, pulmonary system, gastrointestinal system, urinary system and lymphatic system. Accordingly, in some embodiments, release of one or more nutraceutical agents 112 from an administration form at one or more sites associated with an individual 108 may be controlled through selection of materials that degrade under conditions present at the desired site of release. For example, for release in the stomach, one or more nutraceutical agents 112 may be packaged into an administration form that degrades when exposed to acidic conditions. In other examples, one or more nutraceutical agents 112 may be released in the gastrointestinal tract by preparing an administration form that is acid resistant but that degrades under basic conditions. Numerous methods are known that may be used to release one or more nutraceutical agents 112 at one or more sites associated with an individual 108.

At operation 904, the packaging operation 230 may include packaging at least one of the two or more nutraceutical agents in response to a sustained release profile. In some embodiments, one or more packaging units 114 may package at least one of the two or more nutraceutical agents 112 in response to a sustained release profile. In some embodiments, one or more nutraceutical agents 112 may be packaged with a carrier that may include a time-delay or time-release material known in the art, such as glyceryl monostearate or glyceryl distearate alone or with a wax, ethylcellulose, hydroxypropylmethylcellulose, methylmethacrylate and the like. Additionally, in some embodiments, one or more nutraceutical agents 112 may be administered using a sustained-release system, such as semipermeable matrices of solid hydrophobic polymers containing the one or more nutraceutical agents 112. Various sustained-release materials are known and have been described. For example, sustained-release capsules may, depending on their chemical composition, release one or more nutraceutical agents 112 for a few weeks up to over 100 days. Numerous additional sustained-release formulations are known and have been described (i.e., U.S. Pat. Nos. 7,041,670; 7,041,317; 6,709,676; herein incorporated by reference).

At operation 906, the packaging operation 230 may include packaging the two or more nutraceutical agents in storage material. In some embodiments, one or more packaging units 114 may package the two or more nutraceutical agents 112 in storage material. Two or more nutraceutical agents 112 may be packaged in numerous types of storage material. Examples of storage material include, but are not limited to, containers, boxes, ampoules, vials, syringes, and the like. In some embodiments, storage material includes advertising. In some embodiments, storage material includes instructions for administration. Such instructions may include time for administration, route of administration, the name of the individual 108 to whom the two or more nutraceutical agents 112 are to be administered, the identity of the two or more nutraceutical agents 112, the dosage of the two or more nutraceutical agents 112, appropriate buffers for suspension of the two or more nutraceutical agents 112, the source of the two or more nutraceutical agents 112, the date when the two or more nutraceutical agents 112 were packaged, the date when the two or more nutraceutical agents 112 were manufactured, the expiration date of the two or more nutraceutical agents 112, and the like.

At operation 908, the packaging operation 230 may include labeling at least one of the two or more nutraceutical agents. In some embodiments, one or more packaging units 114 may label at least one of the two or more nutraceutical agents 112. In some embodiments, one or more packaging units 114 may place a label directly on at least one of the two or more nutraceutical agents 112. Numerous methods may be used to label at least one of the two or more nutraceutical agents 112. For example, in some embodiments, one or more labeling units may stamp an indented label into at least one of the two or more nutraceutical agents 112. In some embodiments, one or more packaging units 114 may stamp a label onto at least one of the two or more nutraceutical agents 112 through use of one or more edible dyes. Such labels may include numerous types of information. For example, such labels may indicate the manufacturer of at least one of the two or more nutraceutical agents 112, the date of manufacture, the date of packaging, the dosage, the route of administration, and the like. Such labels may be in substantially any language. In some embodiments, at least one label may be a bar code.

At operation 910, the packaging operation 230 may include labeling storage material containing the two or more nutraceutical agents. In some embodiments, one or more packaging units 114 may label storage material containing the two or more nutraceutical agents 112. In some embodiments, storage material may be labeled with advertising. In some embodiments, storage material may be labeled with instructions for administration. Such instructions may include time for administration, route of administration, the name of the individual 108 to whom the two or more nutraceutical agents 112 are to be administered, the identity of the two or more nutraceutical agents 112, the dosage of the two or more nutraceutical agents 112, appropriate buffers for suspension of the two or more nutraceutical agents 112, the source of the two or more nutraceutical agents 112, the date when the two or more nutraceutical agents 112 were packaged, the date when the two or more nutraceutical agents 112 were manufactured, the expiration date of the two or more nutraceutical agents 112, and the like.

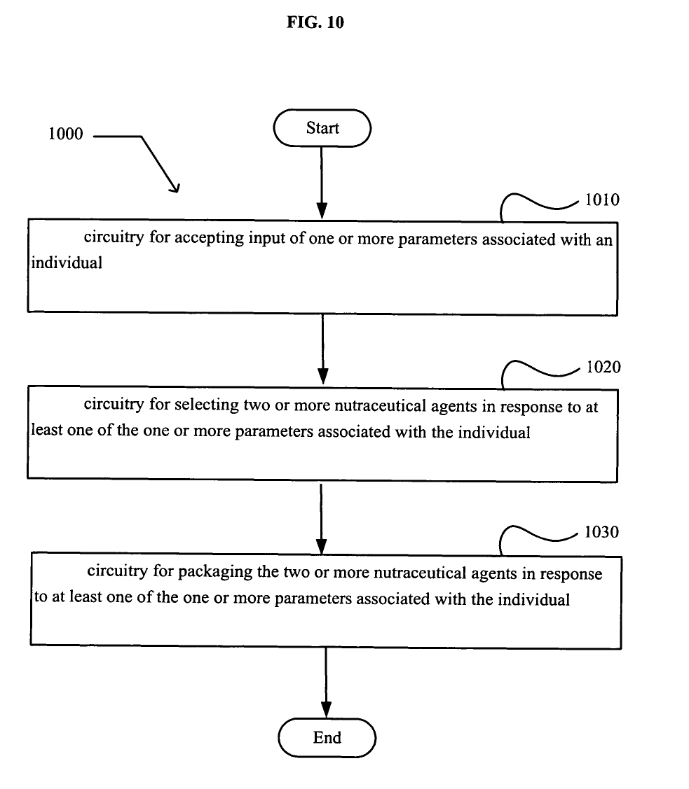

FIG. 10 illustrates a system 1000 representing examples of circuitry that is related to systems for individualized nutraceutical selection and packaging. In FIG. 10 and in following figures that include various examples of circuitry that is related to operations used during performance of a method, discussion and explanation may be provided with respect to the above-described example of FIG. 1, and/or with respect to other examples and contexts. However, it should be understood that the circuitry may be assembled in a number of other environments and contexts, and/or modified versions of FIG. 1. Also, although various circuitry is presented in the sequence(s) illustrated, it should be understood that circuitry may be assembled in other configurations than those which are illustrated.

After a start operation, the system 1000 includes a circuitry block 1010 that includes circuitry for accepting input of one or more parameters associated with an individual. In some embodiments, the circuitry may be used to accept input 104 of one or more parameters 106 associated with an individual 108. In some embodiments, the circuitry may be included within one or more accepting units 102 that accept input 104 of one or more parameters 106 associated with an individual 108.

In some embodiments, an individual 108 may be a human. In some embodiments, an individual 108 may be a non-human animal. Examples of such non-human animals include, but are not limited to, domestic pets such as dogs, cats, horses, potbelly pigs, ferrets, rodents, reptiles, amphibians, and the like. Non-human animals also include animals that include, but are not limited to, cattle, sheep, goats, chickens, pigs, and the like. Accordingly, the systems and methods described herein may be used in association with substantially any human and/or non-human animal.

Numerous parameters 106 may be associated with an individual 108. Such parameters 106 may include, but are not limited to, physical characteristics, metabolic characteristics, financial characteristics, and the like. Examples of parameters 106 include, an individual's height, weight, gender, kidney function, liver function, level of physical fitness, age, allergic response, metabolic level (i.e., resting metabolic rate and/or activity-related metabolic rate), disease state, body fat percentage, personal health habits (i.e., smoking, alcohol consumption, diet, illegal drug use, and the like), family health history, insurance coverage, food supplement usage, nutraceutical usage, non-prescription drug use, prescription drug use, pregnancy status, and the like.

Numerous technologies may be used to provide input 104 that include one or more parameters 106 associated with an individual 108. Examples of such technologies include, but are not limited to, hardwired input 104, wireless input 104, computer input 104, telephonic input 104, internet based input 104, intranet based input 104, digital input 104, analog input 104, input 104 from a human, input 104 from a palm held organizer, input 104 from a personal digital assistant, input 104 from a web enabled cellular telephone, and the like. In some embodiments, one or more accepting units 102 accept input 104 from one source. In some embodiments, one or more accepting units 102 accept input 104 from more than one source. For example, in some embodiments, an accepting unit 102 may accept input 104 from an insurance company, a physician, a pharmacist, a clinical laboratory and a nutraceutical company. In some embodiments, input 104 may be associated with a physician input 104, a pharmacist input 104, a patient input 104, a machine input 104 and/or substantially any combination thereof.

In some embodiments, an accepting unit 102 may include an input device. For example, in some embodiments, an accepting unit 102 may include an interface, such as a keyboard, touch-screen and/or the like, where parameters 106 associated with an individual 108 may be input 104 directly into the accepting unit 102. In some embodiments, an accepting unit 102 may lack an interface where parameters 106 associated with an individual 108 may be directly input 104 into the accepting unit 102. In some embodiments, an accepting unit 102 may accept input 104 of one or more parameters 106 associated with an individual 108 from one or more locations that are remote from the accepting unit 102. For example, in some embodiments, an accepting unit 102 may accept input 104 from a wireless device, the internet, an intranet, a telephone, a palm held organizer, input 104 from a personal digital assistant, input 104 from a web enabled cellular telephone, and the like.

After a start operation, the system 1000 includes a circuitry block 1020 that includes circuitry for selecting two or more nutraceutical agents in response to at least one of the one or more parameters associated with the individual. In some embodiments, the circuitry may be used to select two or more nutraceutical agents 112 in response to at least one of the one or more parameters 106 associated with the individual 108. In some embodiments, the circuitry may be included within one or more selecting units 110 that can be used to select two or more nutraceutical agents 112 in response to at least one of the one or more parameters 106 associated with the individual 108. In some embodiments, one or more selecting units 110 may select one or more first nutraceutical agents 112 in response to at least one of the one or more parameters 106 associated with an individual 108 and select one or more second nutraceutical agents 112 based on the identity of the one or more first nutraceutical agents 112 selected. For example, in some embodiments, one or more selecting units 110 may select the first and second nutraceutical agents 112 to act synergistically with each other when administered to an individual 108. In some embodiments, one or more selecting units 110 may select the first and second nutraceutical agents 112 so that they do not contraindicate each other when administered to an individual 108. Nutraceutical agents 112 may be selected in response to numerous parameters 106.

After a start operation, the system 1000 includes a circuitry block 1030 that includes circuitry for packaging the two or more nutraceutical agents in response to at least one of the one or more parameters associated with the individual. In some embodiments, the circuitry may be used to package the two or more nutraceutical agents 112 in response to at least one of the one or more parameters 106 associated with the individual 108. In some embodiments, the circuitry may be included within one or more packaging units 114 that can be used to package the two or more nutraceutical agents 112 in response to at least one of the one or more parameters 106 associated with the individual 108.

Numerous types of packaging units 114 may be used to package two or more nutraceutical agents 112. In some embodiments, one packaging unit 114 is used to package two or more nutraceutical agents 112. In some embodiments, one or more packaging units 114 are used to package two or more nutraceutical agents 112. In some embodiments, two or more packaging units 114 are used to package two or more nutraceutical agents 112. In some embodiments, a first packaging unit 114 may package one or more first nutraceutical agents 112, a second packaging unit 114 may package one or more second nutraceutical agents 112, and a third packaging unit 114 may package the one or more first nutraceutical agents 112 and one or more second nutraceutical agents 112 together. In some embodiments, one packaging unit 114 may package the two or more nutraceutical agents 112. In some embodiments, one or more packaging units 114 may formulate two or more nutraceutical agents 112 for administration to an individual 108. In some embodiments, one or more packaging units 114 may package two or more preformulated nutraceutical agents 112 for administration to an individual 108. For example, in some embodiments, one or more packaging units 114 may package two or more commercially available nutraceutical preparations to provide for single administration to an individual 108. In some embodiments, one or more packaging units 114 may package two or more preformulated tablets containing the two or more nutraceutical agents 112 into a single capsule for administration to an individual 108. In some embodiments, one or more packaging units 114 may wrap a second nutraceutical agent 112 around a first nutraceutical agent 112 through use of a biocompatible and dissolvable wrapper to produce an administration form having the first and second nutraceutical agents 112 in concentric orientation relative to each other. In some embodiments, one or more packaging units 114 may package two or more nutraceutical agents 112 into a compartmentalized capsule.

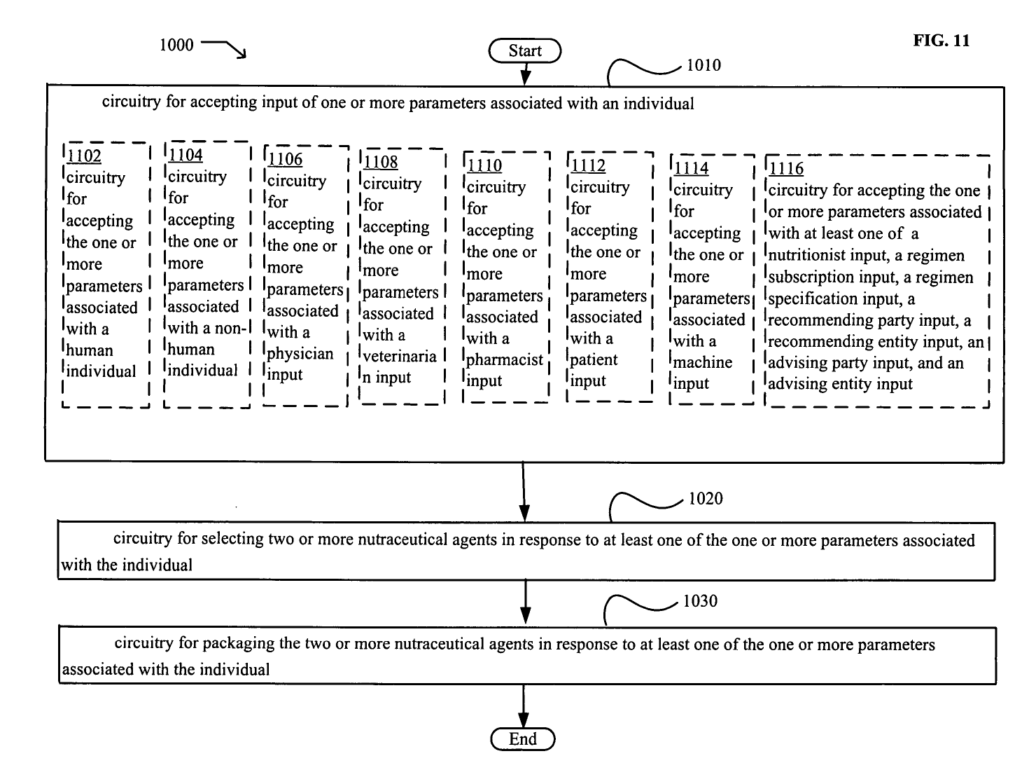

FIG. 11 illustrates alternative embodiments of the system 1000 of FIG. 10. FIG. 11 illustrates example embodiments where the circuitry block 1010 may include at least one additional circuitry block 1102, circuitry block 1104, circuitry block 1106, circuitry block 1108, circuitry block 1110, circuitry block 1112, circuitry block 1114, and/or circuitry block 1116.

At circuitry block 1102, the circuitry block 1010 may include circuitry for accepting the one or more parameters associated with a human individual. In some embodiments, one or more accepting units 102 may include circuitry for accepting the one or more parameters 106 associated with a human individual 108. In some embodiments, the one or more parameters 106 may include physical characteristics, metabolic characteristics, financial characteristics, and substantially any combination thereof. In some embodiments, such parameters 106 may include, alone or in combination and not limited to, an individual's height, weight, gender, kidney function, liver function, level of physical fitness, age, allergic response, metabolic level (i.e., resting metabolic rate and/or activity-related metabolic rate), disease state, body fat percentage, personal habits (i.e., smoking, alcohol consumption, diet, illegal drug use, and the like), family health history, insurance coverage, food supplement usage, physical activities, sleep schedule, activity level, occupation, nutraceutical usage, non-prescription drug use, prescription drug use, pregnancy status, predisposition toward the development of a malady, genotype, phenotype, genetic predisposition, administration form of a nutraceutical agent, mode of administration, time of administration, administration schedule, exposure to pathogens, potential exposure to pathogens, exposure to toxins, potential exposure to toxins, and the like. For example, in some embodiments, one or more parameters 106 associated with a human child may be input 104. Accordingly, such parameters 106 may provide for selection of one or more nutraceutical agents 112 that may be administered to a human child. In other embodiments, such parameters 106 may provide for selection against one or more nutraceutical agents 112 that should not be administered to a human child. Accordingly, in some embodiments, an input 104 may provide for the selection of one or more nutraceutical agents 112. However, in other embodiments, an input 104 may provide for selection against one or more nutraceutical agents 112. In some embodiments, parameters 106 may be input 104 that relate to environmental factors such as, time, temperature, elevation, humidity, events, activities and the like. For example, an input 104 may include parameters 106 related to an individual 108 who is a mountain climber. Accordingly, one or more nutraceutical agents 112 may be selected that will not vaporize under lessened atmospheric pressure, that will not freeze, and/or that will not break. In some embodiments, one or more parameters 106 may be input 104 that relate to administration form and mode of administration of the one or more nutraceutical agents 112 to the individual 108. For example, in some embodiments, one or more parameters 106 may be input 104 that indicate that the individual 108 prefers to orally ingest nutraceutical agents 112. In some embodiments, one or more parameters 106 may be input 104 that indicate that the individual 108 is to ingest two or more nutraceutical agents 112 within a given time period. Accordingly, in some embodiments, an input 104 may be associated with the selection of two or more nutraceutical agents 112 that are compatible with each other and/or that do not contraindicate each other. In some embodiments, an input 104 may be associated with the selection of two or more nutraceutical agents 112 that act in a synergistic manner when administered to an individual 108.

At circuitry block 1104, the circuitry block 1010 may include circuitry for accepting the one or more parameters associated with a non-human individual. In some embodiments, one or more accepting units 102 may include circuitry for accepting the one or more parameters 106 associated with a non-human individual 108. Examples of such non-human animals include, but are not limited to, domestic pets such as dogs, cats, horses, potbelly pigs, ferrets, rodents, reptiles, amphibians, and the like. Non-human animals may also be animals that include, but are not limited to, cattle, sheep, goats, chickens, pigs, and the like. Accordingly, in some embodiments, the methods and/or systems described herein may be used for veterinary purposes. In some embodiments, the one or more parameters 106 may include physical characteristics, metabolic characteristics, financial characteristics (such as valuation of the non-human animal), and substantially any combination thereof. In some embodiments, such parameters 106 may include, alone or in combination and not limited to, a non-human individual's height, weight, gender, kidney function, liver function, level of physical fitness, age, allergic response, metabolic level (i.e., resting metabolic rate and/or activity-related metabolic rate), disease state, body fat percentage, health history, insurance coverage, food supplement usage, physical activities, sleep schedule, activity level, nutraceutical usage, non-prescription drug use, prescription drug use, pregnancy status, predisposition toward the development of a malady, genotype, phenotype, genetic predisposition, administration form, mode of administration, exposure to pathogens, potential exposure to pathogens, exposure to toxins, potential exposure to toxins, and the like. For example, in some embodiments, parameters 106 associated with an infant non-human individual 108 may be input 104. Accordingly, such parameters 106 may provide for selection of one or more nutraceutical agents 112 that may be administered to an infant non-human individual 108. In other embodiments, such parameters 106 may provide for selection against one or more nutraceutical agents 112 that should not be administered to an infant non-human individual 108. Accordingly, in some embodiments, an input 104 may provide for the selection of one or more nutraceutical agents 112. However, in other embodiments, an input 104 may provide for selection against one or more nutraceutical agents 112. In some embodiments, parameters 106 may be input 104 that relate to environmental factors surrounding the non-human individual 108 that include time, temperature, elevation, humidity, events, activities and the like. In some embodiments, one or more parameters 106 may be input 104 that relate to administration form and mode of administration of the one or more nutraceutical agents 112 to the non-human individual 108. For example, in some embodiments, one or more parameters 106 may be input 104 that indicate that one or more nutraceutical agents 112 should be administered to the non-human individual 108 orally. In some embodiments, one or more parameters 106 may be input 104 that indicate that the non-human individual 108 is to ingest two or more nutraceutical agents 112 within a given time period. Accordingly, in some embodiments, an input 104 may be associated with the selection of two or more nutraceutical agents 112 that are compatible with each other and/or that do not contraindicate each other. In some embodiments, an input 104 may be associated with the selection of two or more nutraceutical agents 112 that act in a synergistic manner when administered to a non-human individual 108.

At circuitry block 1106, the circuitry block 1010 may include circuitry for accepting the one or more parameters associated with a physician input. In some embodiments, one or more accepting units 102 may include circuitry for accepting the one or more parameters 106 associated with a physician input 104. In some embodiments, one or more physicians may input 104 one or more parameters 106 associated with an individual 108. In some embodiments, one or more parameters 106 may be input 104 by one or more physicians and one or more other sources. Other sources of input 104 include, but are not limited to, veterinarian input 104, pharmacist input 104, patient input 104, machine input 104, nutritionist input 104, and the like. In some embodiments, one or more physicians may examine the individual 108 and input 104 one or more parameters 106 associated with the individual 108 that are related to the examination. For example, one or more physicians may input 104 one or more parameters 106 associated with an individual's heart rate, skin condition, allergy status, sleep status, and the like. In some embodiments, one or more physicians may input 104 one or more parameters 106 associated with an individual 108 without ever seeing the individual 108. For example, in some embodiments, one or more physicians may review a medical chart associated with the individual 108 and input 104 parameters 106 based on the information contained in the medical chart. In some embodiments, one or more physicians may input 104 parameters 106 associated with an individual 108 from the physician's memory. In some embodiments, one or more physicians may input 104 parameters 106 associated with an individual 108 following consultation with a database and/or other source of information. In some embodiments, one or more physicians may input 104 parameters 106 associated with an individual 108 directly through use of a keyboard, a touch-screen, and the like. In some embodiments, one or more physicians may input 104 parameters 106 associated with an individual 108 remotely through use of numerous technologies that include, input 104 from a wireless device, the internet, an intranet, a telephone, a palm held organizer, input 104 from a personal digital assistant, input 104 from a web enabled cellular telephone, and the like.

At circuitry block 1108, the circuitry block 1010 may include circuitry for accepting the one or more parameters associated with a veterinarian input. In some embodiments, one or more accepting units 102 may include circuitry for accepting the one or more parameters 106 associated with a veterinarian input 104. In some embodiments, one or more veterinarians may input 104 one or more parameters 106 associated with a non-human individual 108. In some embodiments, one or more parameters 106 may be input 104 by one or more veterinarians and one or more other sources. Other sources of input 104 include, but are not limited to, physician input 104, pharmacist input 104, patient input 104, machine input 104, nutritionist input 104, and the like. In some embodiments, one or more veterinarians may examine a non-human individual 108 and input 104 one or more parameters 106 associated with the non-human individual 108 that are related to the examination. For example, one or more veterinarians may input 104 one or more parameters 106 associated with a non-human individual's heart rate, skin condition, allergy status, sleep status, and the like. In some embodiments, one or more veterinarians may input 104 one or more parameters 106 associated with a non-human individual 108 without ever seeing the non-human individual 108. For example, in some embodiments, one or more veterinarians may review a medical chart associated with the non-human individual 108 and input 104 parameters 106 based on the information contained in the medical chart. In some embodiments, one or more veterinarians may input 104 parameters 106 associated with a non-human individual 108 from the veterinarian's memory. In some embodiments, one or more veterinarians may input 104 parameters 106 associated with a non-human individual 108 following consultation with a database and/or other source of information. In some embodiments, one or more veterinarians may input 104 parameters 106 associated with a non-human individual 108 directly through use of a keyboard, a touch-screen, and the like. In some embodiments, one or more veterinarians may input 104 parameters 106 associated with a non-human individual 108 remotely through use of numerous technologies that include, input 104 from a wireless device, the internet, an intranet, a telephone, a palm held organizer, input 104 from a personal digital assistant, input 104 from a web enabled cellular telephone, and the like.

At circuitry block 1110, the circuitry block 1010 may include circuitry for accepting the one or more parameters associated with a pharmacist input. In some embodiments, one or more accepting units 102 may include circuitry for accepting the one or more parameters 106 associated with a pharmacist input 104. In some embodiments, one or more pharmacists may input 104 one or more parameters 106 associated with an individual 108. In some embodiments, one or more parameters 106 may be input 104 by one or more pharmacists and one or more other sources. Other sources of input 104 include, but are not limited to, physician input 104, veterinarian input 104, patient input 104, machine input 104, nutritionist input 104, and the like. In some embodiments, one or more pharmacists may consult with an individual 108 and input 104 one or more parameters 106 associated with the individual 108 that are related to the consultation. For example, one or more pharmacists may input 104 one or more parameters 106 associated with an individual's heart rate, skin condition, allergy status, sleep status, and the like. In some embodiments, one or more pharmacists may input 104 one or more parameters 106 associated with an individual 108 without ever seeing the individual 108. For example, in some embodiments, one or more pharmacists may receive information associated with the individual 108 and input 104 parameters 106 based on the received information. In some embodiments, one or more pharmacists may input 104 parameters 106 associated with an individual 108 from the pharmacist's memory. In some embodiments, one or more pharmacists may input 104 parameters 106 associated with an individual 108 following consultation with a database and/or other source of information. In some embodiments, one or more pharmacists may input 104 parameters 106 associated with an individual 108 directly through use of a keyboard, a touch-screen, and the like. In some embodiments, one or more pharmacists may input 104 parameters 106 associated with an individual 108 remotely through use of numerous technologies that include, input 104 from a wireless device, the internet, an intranet, a telephone, a palm held organizer, input 104 from a personal digital assistant, input 104 from a web enabled cellular telephone, and the like.

At circuitry block 1112, the circuitry block 1010 may include circuitry for accepting the one or more parameters associated with a patient input. In some embodiments, one or more accepting units 102 may include circuitry for accepting the one or more parameters 106 associated with a patient input 104. In some embodiments, a patient may input 104 one or more parameters 106 associated with the patient. In some embodiments, one or more parameters 106 may be input 104 by the patient and one or more other sources. Other sources of input 104 include, but are not limited to, physician input 104, pharmacist input 104, patient input 104, machine input 104, nutritionist input 104, and the like. In some embodiments, a patient may input 104 one or more parameters 106 associated with the patient's heart rate, skin condition, allergy status, sleep status, and the like. In some embodiments, a patient may input 104 parameters 106 associated with the patient following consultation with a database and/or other source of information. In some embodiments, a patient may input 104 parameters 106 associated with the patient directly through use of a keyboard, a touch-screen, and the like. In some embodiments, a patient may input 104 parameters 106 associated with the patient remotely through use of numerous technologies that include, input 104 from a wireless device, the internet, an intranet, a telephone, a palm held organizer, input 104 from a personal digital assistant, input 104 from a web enabled cellular telephone, and the like. In some embodiments, a patient may input 104 parameters 106 associated with nutraceutical agents 112 that are being administered to the patient. In some embodiments, a patient may input 104 parameters 106 associated with one or more times of administration of one or more nutraceutical agents 112.

At circuitry block 1114, the circuitry block 1010 may include circuitry for accepting the one or more parameters associated with a machine input. In some embodiments, one or more accepting units 102 may include circuitry for accepting the one or more parameters 106 associated with a machine input 104. In some embodiments, the one or more parameters 106 may include physical characteristics, metabolic characteristics, financial characteristics, and substantially any combination thereof. In some embodiments, such parameters 106 may include, alone or in combination and not limited to, an individual's height, weight, gender, kidney function, liver function, level of physical fitness, age, allergic response, metabolic level (i.e., resting metabolic rate and/or activity-related metabolic rate), disease state, body fat percentage, personal habits (i.e., smoking, alcohol consumption, diet, illegal drug use, and the like), family health history, insurance coverage, food supplement usage, physical activities, sleep schedule, activity level, occupation, nutraceutical usage, non-prescription drug use, prescription drug use, pregnancy status, predisposition toward the development of a malady, genotype, phenotype, genetic predisposition, administration form of a nutraceutical agent, mode of administration, time of administration, administration schedule, exposure to pathogens, potential exposure to pathogens, exposure to toxins, potential exposure to toxins, and the like. For example, in some embodiments, one or more parameters 106 associated with a human child may be input 104. Accordingly, such parameters 106 may provide for selection of one or more nutraceutical agents 112 that may be administered to a human child. In other embodiments, such parameters 106 may provide for selection against one or more nutraceutical agents 112 that should not be administered to a human child. Accordingly, in some embodiments, an input 104 may provide for the selection of one or more nutraceutical agents 112. However, in other embodiments, an input 104 may provide for selection against one or more nutraceutical agents 112. In some embodiments, parameters 106 may be input 104 that relate to environmental factors such as, time, temperature, elevation, humidity, events, activities and the like. For example, an input 104 may include parameters 106 related to an individual 108 who is a mountain climber. Accordingly, one or more nutraceutical agents 112 may be selected that will not vaporize under lessened atmospheric pressure, that will not freeze, and/or that will not break. In some embodiments, one or more parameters 106 may be input 104 that relate to administration form and mode of administration of the one or more nutraceutical agents 112 to the individual 108. For example, in some embodiments, one or more parameters 106 may be input 104 that indicate that the individual 108 prefers to orally ingest nutraceutical agents 112. In some embodiments, one or more parameters 106 may be input 104 that indicate that the individual 108 is to ingest two or more nutraceutical agents 112 within a given time period. Accordingly, in some embodiments, an input 104 may be associated with the selection of two or more nutraceutical agents 112 that are compatible with each other and/or that do not contraindicate each other. In some embodiments, an input 104 may be associated with the selection of two or more nutraceutical agents 112 that act in a synergistic manner when administered to an individual 108. In some embodiments, the machine is a diagnostic machine that has been utilized during examination of the individual 108.

At circuitry block 1116, the circuitry block 1010 may include circuitry for accepting the one or more parameters associated with at least one of a nutritionist input, a regimen subscription input, a regimen specification input, a recommending party input, a recommending entity input, an advising party input, or an advising entity input. In some embodiments, one or more accepting units 102 may include circuitry for accepting the one or more parameters associated with at least one of a nutritionist input 104, a regimen subscription input 104, a regimen specification input 104, a recommending party input 104, a recommending entity input 104, an advising party input 104, and an advising entity input 104. In some embodiments, input 104 may include one or more parameters 106 associated with an individual 108. In some embodiments, input 104 may include one or more parameters associated with an individual 108 that are input 104 by one or more sources. Other sources of input 104 include, but are not limited to, physician input 104, veterinarian input 104, patient input 104, machine input 104, pharmacist input 104, regimen subscription input 104, regimen specification input 104, recommending party input 104, recommending entity input 104, advising party input 104, advising entity input 104, and the like. In some embodiments, one or more sources of input 104 may consult with an individual 108 and input 104 one or more parameters 106 associated with the individual 108 that are related to the consultation. For example, one or more nutritionists may input 104 one or more parameters 106 associated with an individual's heart rate, skin condition, allergy status, sleep status, and the like. In some embodiments, one or more nutritionists may input 104 one or more parameters 106 associated with an individual 108 without ever seeing the individual 108. For example, in some embodiments, one or more nutritionists may receive information associated with the individual 108 and input 104 parameters 106 based on the received information. In some embodiments, one or more nutritionists may input 104 parameters 106 associated with an individual 108 from the nutritionist's memory. In some embodiments, one or more nutritionists may input 104 parameters 106 associated with an individual 108 following consultation with a database and/or other source of information. In some embodiments, one or more nutritionists may input 104 parameters 106 associated with an individual 108 directly through use of a keyboard, a touch-screen, and the like. In some embodiments, one or more nutritionists may input 104 parameters 106 associated with an individual 108 remotely through use of numerous technologies that include, input 104 from a wireless device, the internet, an intranet, a telephone, a palm held organizer, input 104 from a personal digital assistant, input 104 from a web enabled cellular telephone, and the like. Input 104 may be associated with grocery stores, food supplement stores, personal trainers, coaches, clinics, hospitals, dental offices, veterinary offices, and the like.

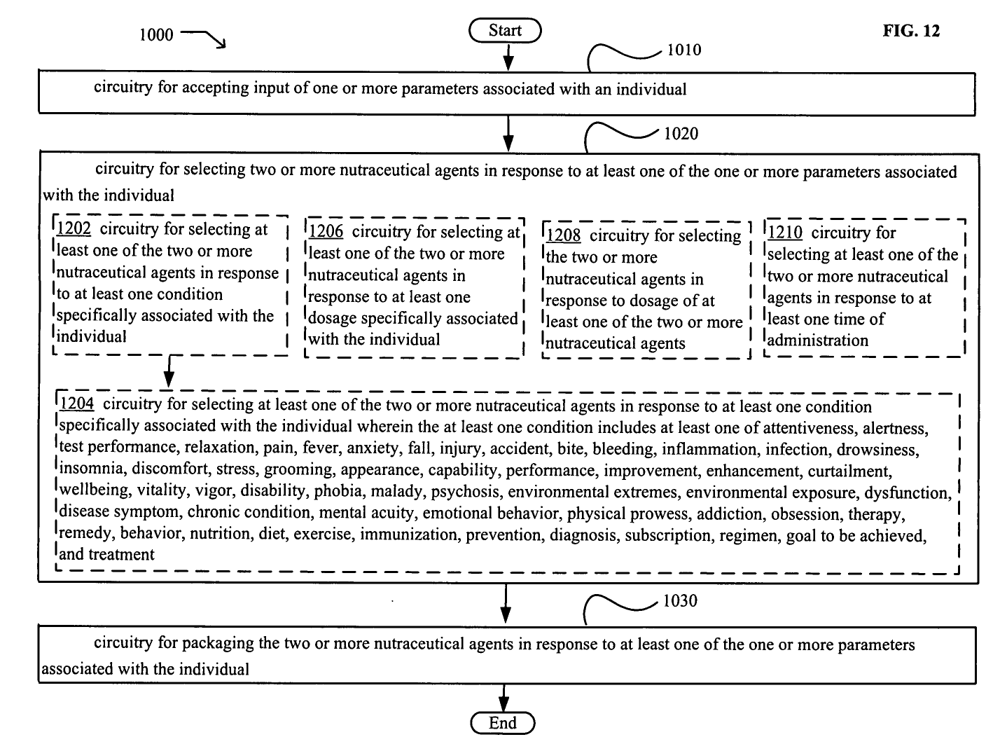

FIG. 12 illustrates alternative embodiments of the system 1000 of FIG. 10. FIG. 12 illustrates example embodiments where circuitry block 1020 may include at least one additional circuitry block 1202, circuitry block 1204, circuitry block 1206, circuitry block 1208, and/or circuitry block 1210.

At circuitry block 1202, the circuitry block 1020 may include circuitry for selecting at least one of the two or more nutraceutical agents in response to at least one condition specifically associated with the individual. In some embodiments, one or more selecting units 110 may include circuitry for selecting at least one of the two or more nutraceutical agents 112 in response to at least one condition specifically associated with the individual 108.

In some embodiments, a condition specifically associated with an individual 108 may be an existing condition. In some embodiments, an existing condition is a medical condition. Examples of such medical conditions include, but are not limited to, viral infection, bacterial infection, fungal infection, diabetes, arthritis, gastrointestinal maladies, cancer, allergic responses, psychological disorders, osteoporosis, Alzheimer's disease, asthma, chronic fatigue syndrome, epilepsy, heart disease, hemochromatosis, hepatitis, stroke, food intolerance, and the like in substantially any combination. Accordingly, one or more nutraceutical agents 112 may be selected to reduce or ameliorate the symptoms of a condition and/or to treat the condition directly. Numerous nutraceutical agents 112 that may be selected in response to a condition are known (i.e., Roberts et al., Nutraceuticals: The Complete Encyclopedia of Supplements, Herbs, Vitamins, and Healing Foods, 1^{st} Edition, Perigee Trade (2001); Rapport and Lockwood, Nutraceuticals, Pharmaceutical Press (2001); Wildman, Handbook of Nutraceuticals and Functional Foods, CRC Press, 1^{st} Edition, (2000); Eskin, Dictionary of Nutraceuticals and Functional Foods (Functional Foods and Nutraceuticals), CRC Press, (2005); The PDR Family Guide to Nutritional Supplements: An Authoritative A-to-Z Resource on the 100 Most Popular Nutritional Therapies and Nutraceuticals; Ballantine Books, 1^{st} Edition, (2001); Susan G. Wynn, Emerging Therapies: Using Herbs and Nutraceuticals for Small Animals, American Animal Hospital Assn Press (1999); U.S. Pat. Nos. 7,045,159; 7,049,433; 7,041,840; 6,979,679; 6,962,720; The Merck Index, 13^{th} Edition, An Encyclopedia of Chemicals, Drugs, and Biologicals, Merck & Co. Inc., Whitehouse Station, N.J. (2001); Mosby's Drug Guide, Mosby, Inc., St. Louis, Mo. (2004); Remington: The Science and Practice of Pharmacy, 20^{th} Edition, Lippincott Williams & Wilkins, Philadelphia, Pa. (2000); Physicians' Desk Reference, 58^{th} Edition, Thompson, PDR, Montvale, N.J. (2004); U.S. Pat. No. 6,773,721; herein incorporated by reference).

In some embodiments, a condition specifically associated with an individual 108 may be a past condition. For example, one or more nutraceutical agents 112 may be selected such that a condition, such as a medical condition, that an individual 108 was treated for in the past will be disallowed from reoccurring or the condition, or symptoms of the condition, may be reduced or minimized if the condition were to reoccur in the individual 108. For example, in some embodiments, one or more nutraceutical agents 112 may be selected to prevent or reduce the consequences of a heart attack that may reoccur in an individual 108. In some embodiments, one or more nutraceutical agents 112 may be selected to prevent or reduce the consequences of an epileptic seizure in an individual 108. Accordingly, one or more nutraceutical agents 112 may be selected in response to numerous past conditions associated with the individual 108.

In some embodiments, a condition specifically associated with an individual 108 may be a future condition. For example, one or more nutraceutical agents 112 may be selected such that a condition, such as a medical condition, that an individual 108 is predisposed to developing in the future may be disallowed from occurring or the condition, or symptoms of the condition, may be reduced or minimized if the condition were to occur in the individual 108. Accordingly, one or more nutraceutical agents 112 may be selected in response to numerous future conditions associated with the individual 108. In some embodiments, one or more nutraceutical agents 112 may be selected to prevent the occurrence of a future condition. In some embodiments, one or more nutraceutical agents 112 may be selected in response to conditions that are cyclic. For example, in some embodiments, one or more nutraceutical agents 112 may be selected in response to a woman's menstrual cycle. In other embodiments, one or more nutraceutical agents 112 may be selected in response to a psychological malady, such as depression, that occurs in a cyclic manner. In other embodiments, one or more nutraceutical agents 112 may be selected in response to hormonal changes that are expected to occur in the future, such as menopause.

In some embodiments, a condition specifically associated with an individual 108 may be an event or activity associated with an individual 108. For example, in some embodiments, one or more nutraceutical agents 112 may be selected in response to a condition that is an event associated with an individual 108. For example, in some embodiments, an individual 108 may be expecting to participate in a sporting event. Accordingly, one or more nutraceutical agents 112 may be selected in response to the event such that the one or more agents will not interfere with the performance of the individual 108. In other examples, the one or more nutraceutical agents 112 may be selected to improve performance of the individual 108 in the event. In some embodiments, an individual 108 may expect to give a presentation. Accordingly, one or more nutraceutical agents 112 may be selected that will not interfere with the performance of the individual 108 or that will improve performance of the individual 108 giving the presentation.

In some embodiments, a condition specifically associated with an individual 108 may be related to the environment in which the individual 108 resides or expects to reside. For example, if an individual 108 expects to travel on a boat, one or more nutraceutical agents 112 may be selected that will not contribute to, or that will reduce or ameliorate, motion sickness. In some embodiments, the one or more nutraceutical agents 112 may be selected based on the climactic environment in which an individual 108 resides or expects to reside. For example, one or more nutraceutical agents 112 may be selected based on temperature, humidity, atmospheric pressure, and the like in substantially any combination. In some embodiments, the one or more nutraceutical agents 112 may be selected based on the biological environment in which an individual 108 resides or expects to reside. For example, one or more nutraceutical agents 112 may be selected based on the presence of allergens, pathogens, infectious agents, toxins, organisms and the like in substantially any combination.

In some embodiments, a condition specifically associated with an individual 108 may be a condition known to be associated with the individual 108 or a condition thought to be associated with an individual 108. For example, in some embodiments, one or more nutraceutical agents 112 may be selected that can be used to treat an individual 108 with a diagnosed condition. In other embodiments, one or more nutraceutical agents 112 may be selected that can be administered to an individual 108 with an undiagnosed condition with which the individual 108 was believed to be affected in the in the past, present or future. For example, in some embodiments, 5-hydroxytryptophan, s-adenosylmethionine, St. John's wort, Kava kava, Ginko biloba, melatonin, and/or substantially any combination thereof may be a selected for administration to an individual 108 to reduce or eliminate symptoms associated with depression. In other embodiments, glucosamine and/or chondroitan may be selected for administration to an individual 108 to rebuild cartilage that cushions and protects joints. In some embodiments, lithium may be used to reduce or eliminate symptoms associated with manic/depressive (bipolar) and depressive disorders associated with an individual 108.

At circuitry block 1204, the circuitry block 1020 may include circuitry for selecting at least one of the two or more nutraceutical agents in response to at least one condition specifically associated with the individual wherein the at least one condition includes at least one of attentiveness, alertness, test performance, relaxation, pain, fever, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, goal to be achieved, or treatment. In some embodiments, one or more selecting units 110 may include circuitry for selecting at least one of the two or more nutraceutical agents 112 in response to at least one condition specifically associated with the individual wherein the at least one condition includes at least one of attentiveness, alertness, test performance, relaxation, pain, fever, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, goal to be achieved, and treatment. Accordingly, in some embodiments, at least one of the two or more nutraceutical agents may be selected in response to an existing condition. In some embodiments, at least one of the two or more nutraceutical agents may be selected in reponse to a goal that is to be achieved in the future. Examples of such goals include, but are not limited to, attentiveness, alertness, increased test performance, relaxation, and the like.

At circuitry block 1206, the circuitry block 1020 may include circuitry for selecting at least one of the two or more nutraceutical agents in response to at least one dosage specifically associated with the individual. In some embodiments, one or more selecting units 110 may include circuitry for selecting at least one of the two or more nutraceutical agents 112 in response to at least one dosage specifically associated with the individual 108.

In some embodiments, one or more selecting units 110 may select one or more nutraceutical agents 112 with regard to a volume of one or more of the nutraceutical agents 112. For example, one or more selecting units 110 may select a first nutraceutical agent 112 preferentially over a second nutraceutical agent 112 if the first nutraceutical agent 112 occupies less volume than the second nutraceutical agent 112. In other examples, one or more selecting units 110 may select a first nutraceutical agent 112 preferentially over a second nutraceutical agent 112 if the first nutraceutical agent 112 occupies more volume than the second nutraceutical agent 112. Accordingly, one or more nutraceutical agents 112 may be selected to increase or decrease the volume of the administration form of the one or more nutraceutical agents 112 to promote administration to an individual 108.

In some embodiments, one or more selecting units 110 may select one or more nutraceutical agents 112 with regard to the compatibility of the nutraceutical agents 112 with each other or with the individual 108 at the dosage associated with the individual 108. For example, in some embodiments, one or more nutraceutical agents 112 may be selected that are compatible with each other in response to dosage of at least one of the nutraceutical agents 112. In some embodiments, one or more nutraceutical agents 112 may be selected to act synergistically with each other when administered to an individual 108 at a given dosage. In some embodiments, one or more nutraceutical agents 112 may be selected to avoid synergistic interactions with each other when administered to an individual 108 at a given dosage. In some embodiments, one or more nutraceutical agents 112 may be selected to counteract or reduce any negative side-effects of the one or more nutraceutical agents 112 when they are administered to an individual 108 at a given dosage. In some embodiments, one or more nutraceutical agents 112 may be selected with regard to dosage so that they do not contraindicate additional components, such as nutraceuticals and/or food supplements, ingested by an individual 108. In some embodiments, one or more nutraceutical agents 112 may be selected with regard to the price of the one or more nutraceutical agents 112 with regard to one or more dosages associated with an individual 108. For example, in some embodiments, a nutraceutical agent 112 may be commercially available at two or more dosages that are priced differently. Accordingly, in some embodiments, the one or more nutraceutical agents 112 may be selected to achieve a desired dosage when administered to an individual 108 while reducing or minimizing the price associated with the one or more nutraceutical agents 112.

At circuitry block 1208, the circuitry block 1020 may include circuitry for selecting the two or more nutraceutical agents in response to dosage of at least one of the two or more nutraceutical agents. In some embodiments, one or more selecting units 110 may include circuitry for selecting the two or more nutraceutical agents 112 in response to dosage of at least one of the two or more nutraceutical agents 112.

In some embodiments, one or more nutraceutical agents 112 may be commercially available in preformulated administration forms. Accordingly, in some embodiments, one or more nutraceutical agents 112 may be selected in response to administration forms that are commercially available. For example, in some embodiments, a nutraceutical agent 112 may be commercially available in 100 milligram, 250 milligram, 500 milligram, 750 milligram and 1000 milligram preformulated administration forms. In some instances, an individual 108 may be prescribed or instructed to ingest 750 milligrams of a nutraceutical agent 112. Accordingly, in some embodiments, a 750 milligram administration form of the nutraceutical agent 112 may be selected. In other embodiments, a 250 milligram and a 500 milligram administration form of the nutraceutical agent 112 may be selected. In other embodiments, a 250 milligram and five 100 milligram administration forms of the nutraceutical agent 112 may be selected. Numerous combinations of administration forms may be selected. In some embodiments, administration forms may be selected with regard to price associated with the administration form. For example, in some embodiments, it may be less expensive to achieve a 750 milligram dosage of a nutraceutical agent 112 by combining one 250 milligram administration form with five 100 milligram administration forms than selecting a single 750 milligram administration form.

In some embodiments, one or more nutraceutical agents 112 may be selected with regard to administration forms for administration to an individual 108 over one or more periods of time. For example, it may be desirable to administer 1000 milligrams of a nutraceutical agent 112 to an individual 108 over a ten hour time period. Accordingly, in some embodiments, a single 1000 milligram controlled release administration form may be selected. In other embodiments, ten 100 milligram administration forms may be selected and then packaged to be released at a rate of one 100 milligram administration form per hour over the ten hour period. Accordingly, numerous combinations of administration forms and timed release may be selected.

In some embodiments, one or more selecting units 110 may select one or more nutraceutical agents 112 with regard to one or more volumes of one or more of the nutraceutical agents 112 in the available administration forms. For example, one or more selecting units 110 may select a first nutraceutical agent 112 preferentially over a second nutraceutical agent 112 if the first nutraceutical agent 112 occupies less volume than the second nutraceutical agent 112 with regard to available administration forms. In other examples, one or more selecting units 110 may select a first nutraceutical agent 112 preferentially over a second nutraceutical agent 112 if the first nutraceutical agent 112 occupies more volume than the second nutraceutical agent 112 with regard to available administration forms. Accordingly, one or more nutraceutical agents 112 may be selected to increase or decrease the volume of the one or more nutraceutical agents 112 to promote administration to an individual 108.

In some embodiments, one or more selecting units 110 may select one or more nutraceutical agents 112 with regard to compatibility of the nutraceutical agents 112 with each other and/or with the individual 108 when administered to the individual 108 at dosages corresponding to available administration forms of the nutraceutical agents 112. For example, in some embodiments, one or more nutraceutical agents 112 may be selected in response to administration forms available for the two or more nutraceutical agents 112. In some embodiments, two or more nutraceutical agents 112 may be selected to act synergistically with each other when administered to an individual 108 at available administration forms. In some embodiments, one or more nutraceutical agents 112 may be selected to avoid synergistic interactions with each other when administered to an individual 108 as available administration forms. In some embodiments, one or more nutraceutical agents 112 may be selected to counteract or reduce any negative side-effects of the one or more nutraceutical agents 112 when they are administered to an individual 108 at an available dosage. In some embodiments, one or more nutraceutical agents 112 may be selected with regard to available dosage so that they do not contraindicate additional components, such as nutraceuticals and/or food supplements, ingested by an individual 108. In some embodiments, one or more nutraceutical agents 112 may be selected with regard to the price of the one or more nutraceutical agents 112 with regard to one or more available dosages associated with the one or more nutraceutical agents 112. For example, in some embodiments, a nutraceutical agent 112 may be commercially available at two or more dosages that are priced differently. Accordingly, in some embodiments, the one or more nutraceutical agents 112 may be selected to achieve a desired dosage when administered to an individual 108 while reducing or minimizing the price associated with the one or more nutraceutical agents 112.

At circuitry block 1210, the circuitry block 1020 may include circuitry for selecting at least one of the two or more nutraceutical agents in response to at least one time of administration. In some embodiments, one or more selecting units 110 may include circuitry for selecting at least one of the two or more nutraceutical agents 112 in response to at least one time of administration.

In some embodiments, the at least one time of administration is a time when the one or more nutraceutical agents 112 are to be administered to an individual 108 to provide for release of the one or more nutraceutical agents 112 from the administration form at a specified time following administration. For example, in some embodiments, at least one of the two or more nutraceutical agents 112 may be selected such that it is released from an administration form about one hour after being administered to an individual 108. In other embodiments, a first nutraceutical agent 112 may be selected such that it is released from an administration form about one hour after being administered to an individual 108 and a second nutraceutical agent 112 may be selected such that it is released from an administration form about two hours after being administered to the individual 108. Accordingly, one or more nutraceutical agents 112 may be selected that are released from an administration form at a specified time following administration to an individual 108 and thereupon become functionally available to the individual 108. In some embodiments, two or more incompatible nutraceutical agents 112 may be administered to an individual 108 at the same time without adverse consequences by providing for release of the incompatible nutraceutical agents 112 at different times such that they do not contraindicate each other. In some embodiments, two or more nutraceutical agents 112 that act synergistically may be coadministered to an individual 108 such that they are released at substantially the same time to provide for synergistic action of the two or more nutraceutical agents 112 with regard to the individual 108. Substantially any combination of nutraceutical agents 112, dosages and release times may be selected.

In some embodiments, the at least one time of administration is relative to a time or event preceding or following administration of one or more nutraceutical agents 112 to an individual 108. Accordingly, one or more nutraceutical agents 112 may be selected that are released from an administration form at a relative time following administration to an individual 108 and thereupon become functionally available to the individual 108. For example, in some embodiments, two or more nutraceutical agents 112 may be coadministered to an individual 108 such that a first nutraceutical agent 112 is released from the administration form and a second nutraceutical agent 112 is released from the administration form at a second time that is relative to the time of release of the first nutraceutical agent 112. Accordingly, in some embodiments, two or more incompatible nutraceutical agents 112 may be administered to an individual 108 at the same time without adverse consequences by providing for release of the incompatible nutraceutical agents 112 at different times such that they do not contraindicate each other. In some embodiments, two or more nutraceutical agents 112 that act synergistically may be coadministered to an individual 108 such that they are released at substantially the same time to provide for synergistic action of the two or more nutraceutical agents 112 with regard to the individual 108. In some embodiments, dosages of the two or more nutraceutical agents 112 may be altered in a relative manner. For example, in some embodiments, the dosage of two or more nutraceutical agents 112 may be calibrated relative to time of day. In other embodiments, the dosage of two or more nutraceutical agents 112 may be calibrated relative to hormonal cycles. In other embodiments, the dosage of two or more nutraceutical agents 112 may be calibrated relative to circadian rhythms. Substantially any combination of nutraceutical agents, dosages and release times may be selected relative to a time, event and/or the like.

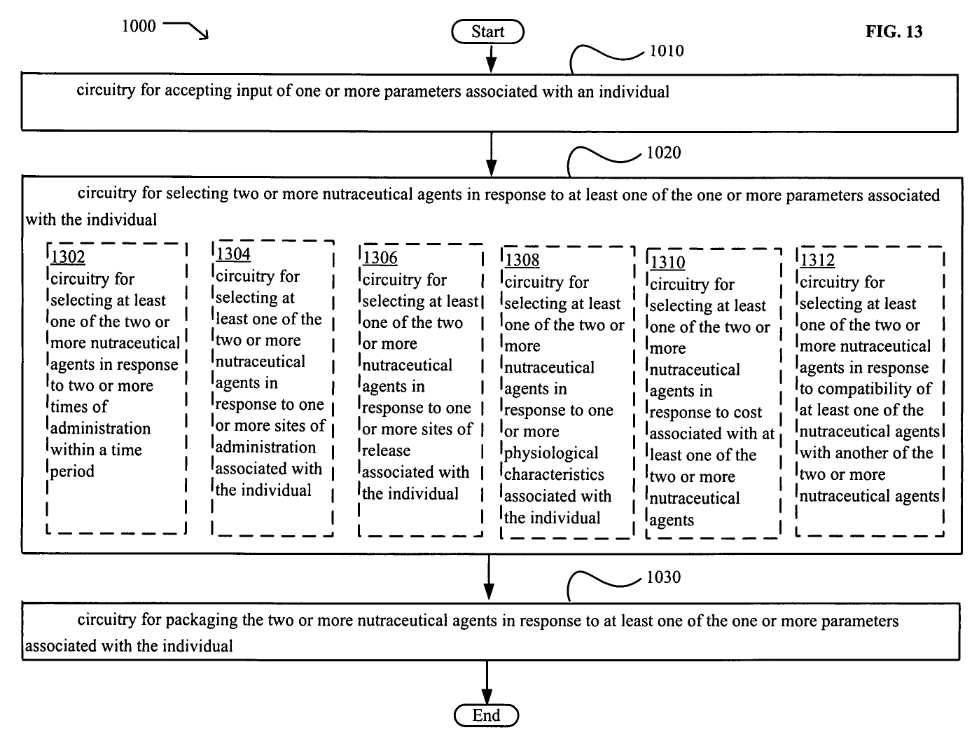

FIG. 13 illustrates alternative embodiments of the system 1000 of FIG. 10. FIG. 13 illustrates example embodiments where the circuitry block 1020 may include at least one additional circuitry block 1302, circuitry block 1304, circuitry block 1306, circuitry block 1308, circuitry block 1310, and/or circuitry block 1312.

At circuitry block 1302, the circuitry block 1020 may include circuitry for selecting at least one of the two or more nutraceutical agents in response to two or more times of administration within a time period. In some embodiments, one or more selecting units 110 may include circuitry for selecting at least one of the two or more nutraceutical agents 112 in response to two or more times of administration within a time period. In some embodiments, a time period is defined as being a discrete amount of time. For example, in some embodiments, a time period may be defined in seconds, minutes, hours, days, months, years and substantially any combination thereof. In some embodiments, a time period may be defined as being an amount of time that is relative to a measurable quantity and/or event. For example, in some embodiments, a time period may be determined based on the concentration of a nutraceutical agent 112 that was previously administered to an individual 108. Accordingly, in some embodiments, a first nutraceutical agent 112 may be administered to an individual 108 and a second nutraceutical agent 112 may be administered to the same individual 108 when the concentration of the first nutraceutical agent 112 associated with the individual 108 either reaches a certain level or decreases to a certain level. Numerous combinations of discrete and/or relative amounts of time may be used during the selection of at least one of two or more nutraceutical agents 112. In some embodiments, at least one of the two or more nutraceutical agents 112 may be selected based on the identity of a second nutraceutical agent 112 that is to be administered to an individual 108 within a time period in which the first nutraceutical agent 112 is still present and/or functionally active in association with an individual 108. For example, in some embodiments, a first nutraceutical agent 112 is selected such that it does not contraindicate a second nutraceutical agent 112 that is to be administered to the individual 108 within a time period when the first and second nutraceutical agents 112 are both present and/or functionally active in association with the individual 108. In some embodiments, the second nutraceutical agent 112 is selected such that it does not contraindicate a first nutraceutical agent 112 that is present and/or functionally active in association with the individual 108. In some embodiments, a first nutraceutical agent 112 is selected such that it will act in a synergistic manner with a second nutraceutical agent 112 that is to be administered to the individual 108 within a time period when the first and second nutraceutical agents 112 are both present and/or functionally active in association with the individual 108. In some embodiments, the second nutraceutical agent 112 is selected such that it will act in a synergistic manner with a first nutraceutical agent 112 that is present and/or functionally active in association with the individual 108.

At circuitry block 1304, the circuitry block 1020 may include circuitry for selecting at least one of the two or more nutraceutical agents in response to one or more sites of administration associated with the individual. In some embodiments, one or more selecting units 110 may include circuitry for selecting at least one of the two or more nutraceutical agents 112 in response to one or more sites of administration associated with the individual 108. One or more nutraceutical agents 112 may be administered at numerous sites associated with an individual 108. Examples of such sites include, but are not limited to, the eyes, ears, nose, skin, mouth, stomach, intestine, rectum, vagina, vascular system, pulmonary system, gastrointestinal system, urinary system and lymphatic system. In some embodiments, one or more nutraceutical agents 112 may be administered at a first site associated with an individual 108 in preference to a second site associated with an individual 108. For example, in some embodiments, it may be desirable to administer a nutraceutical agent 112 that is acid labile by injection into the vascular system in preference to oral administration which may expose the nutraceutical agent 112 to acidic conditions. Accordingly, in some embodiments, one or more nutraceutical agents 112 may be selected based on the physical and chemical characteristics of the one or more nutraceutical agents 112 and where the one or more nutraceutical agents 112 will be administered to an individual 108. In some embodiments, one or more nutraceutical agents 112 may be selected in response to the site of action of the one or more nutraceutical agents 112 on an individual 108. For example, in some embodiments, an adhesive patch may be used to administer one or more nutraceutical agents 112 for the treatment of a malady associated with the skin. In some embodiments, one or more first nutraceutical agents 112 may be selected for administration to a first site associated with an individual 108 and one or more second nutraceutical agents 112 may be selected such that the second nutraceutical agents 112 facilitate administration of the first nutraceutical agents 112, do not contraindicate the first nutraceutical agents 112, act synergistically with the first nutraceutical agents 112, are administered to a second site associated with the individual 108, and/or substantially any combination thereof.

At circuitry block 1306, the circuitry block 1020 may include circuitry for selecting at least one of the two or more nutraceutical agents in response to one or more sites of release associated with the individual. In some embodiments, one or more selecting units 110 may include circuitry for selecting at least one of the two or more nutraceutical agents 112 in response to one or more sites of release associated with the individual 108. In some embodiments, one or more nutraceutical agents 112 may be administered to an individual 108 at a first site and then released from the administration form in which the nutraceutical agents 112 were administered at a second site associated with the individual 108. For example, in some embodiments, one or more nutraceutical agents 112 may be administered to an individual 108 in an oral administration form which can be released in the small intestine of the individual 108. In examples of other embodiments, one or more nutraceutical agents 112 may be released into the vascular system of an individual 108 following transdermal administration of the one or more nutraceutical agents 112 to the individual 108. In some embodiments, two or more nutraceutical agents 112 may be coadministered to an individual 108 such that they are released from their administration forms at two or more separate sites associated with the individual 108. For example, in some embodiments, a first and second nutraceutical agent 112 may be coadministered to an individual 108 such that the first nutraceutical agent 112 is substantially released from the administration form in the upper gastrointestinal tract and the second nutraceutical agent 112 is substantially released from the administration form in the lower gastrointestinal tract. Accordingly, in some embodiments, two or more nutraceutical agents 112 that are incompatible or that would contraindicate each may be coadministered to an individual 108 for release at different sites associated with the individual 108 and/or at different times.

At circuitry block 1308, the circuitry block 1020 may include circuitry for selecting at least one of the two or more nutraceutical agents in response to one or more physiological characteristics associated with the individual. In some embodiments, one or more selecting units 110 may include circuitry for selecting at least one of the two or more nutraceutical agents 112 in response to one or more physiological characteristics associated with the individual 108. Numerous physiological characteristics may be associated with an individual 108. Examples of such characteristics include, but are not limited to, age, gender, disease state, allergic responses, activity-related metabolic rate, resting metabolic rate, liver function, kidney function, weight, body fat percentage, epithelial cell function, lung function, skin function, gastrointestinal tract function, and substantially any combination thereof. Methods to predict drug response and to assess and correlate metabolism to drug dosage are known (i.e., International Publication Numbers.: WO 03/084395 and WO 2005/041105; U.S. Pat. Nos. 6,317,719 and 6,087,090, herein incorporated by reference). Such methods may also be used to predict and to assess and correlate metabolism of a nutraceutical agent 112 by an individual 108. Accordingly, numerous assays may be used to assess the ability of an individual 108 to metabolize one or more nutraceutical agents 112. In some embodiments, enzyme activities may be assessed to determine the ability of an individual 108 to metabolize one or more nutraceutical agents 112. Examples of such enzyme systems and activities that may be assessed include, but are not limited to, the cytochrome P450 monooxygenase system, the flavin-containing monooxygenase system, alcohol dehydrogenase, aldehyde dehydrogenase, monoamine oxidase, cooxidation by peroxidases, NADPH-cytochrome P450 reductase, the presence of reduced (ferrous) cytochrome P450, esterases, amidases, epoxide hydrolase, glutathione S-transferases, mercapturic acid biosynthesis, UDP-Glucoron(os)yltransferases, N-Acetyltransferases, amino acid N-acyl transferases and sulfotransferases. In some embodiments, first and second nutraceutical agents 112 may be effective to treat the same condition associated with an individual 108. However, an individual 108 may be able to metabolize the first nutraceutical agent 112 very quickly but metabolize a second nutraceutical agent 112 more slowly. Accordingly, in some embodiments, the second nutraceutical agent 112 may be selected for administration to the individual 108 to avoid higher relative metabolism of the first nutraceutical agent 112 by the individual 108. In some embodiments, an individual 108 may mount an adverse allergic response to one or more nutraceutical agents 112. Accordingly, one or more nutraceutical agents 112 may be selected to avoid or minimize allergic response to administration of the one or more nutraceutical agents 112 to the individual 108. One or more nutraceutical agents, and combinations of one or more nutraceutical agents, may be selected in response to numerous physiological characteristics associated with an individual 108.

At circuitry block 1310, the circuitry block 1020 may include circuitry for selecting at least one of the two or more nutraceutical agents in response to cost associated with at least one of the two or more nutraceutical agents. In some embodiments, one or more selecting units 110 may include circuitry for selecting the two or more nutraceutical agents 112 in response to cost associated with at least one of the two or more nutraceutical agents 112. In some embodiments, two or more different nutraceutical agents 112 may be used to treat the same or a similar condition associated with an individual 108. In some embodiments, it may be preferable to select a first nutraceutical agent 112 having a lower associated cost over a second nutraceutical agent 112 having a higher associated cost for administration to an individual 108. In other embodiments, it may be preferable to select a first nutraceutical agent 112 having a higher associated cost over a second nutraceutical agent 112 having a lower associated cost for administration to an individual 108. In some embodiments, one or more nutraceutical agents 112 may be selected in response to cost associated with the one or more nutraceutical agents 112 and numerous additional considerations. Such additional considerations include, but are not limited to, allergic response, dosage, effectiveness, interaction with other nutraceutical agents 112 and substantially any combination thereof.

At circuitry block 1312, the circuitry block 1020 may include circuitry for selecting at least one of the two or more nutraceutical agents in response to compatibility of at least one of the nutraceutical agents with another of the two or more nutraceutical agents. In some embodiments, one or more selecting units 110 may include circuitry for selecting at least one of the two or more nutraceutical agents 112 in response to compatibility of at least one of the nutraceutical agents 112 with another of the two or more nutraceutical agents 112. In some embodiments, at least one of the nutraceutical agents 112 is selected that does not interact with another of the two or more nutraceutical agents 112. In some embodiments, at least one of the nutraceutical agents 112 is selected to act in a synergistic manner with another of the two or more nutraceutical agents 112. In some embodiments, at least one of the nutraceutical agents 112 is selected to not contraindicate at least one of the two or more nutraceutical agents 112.

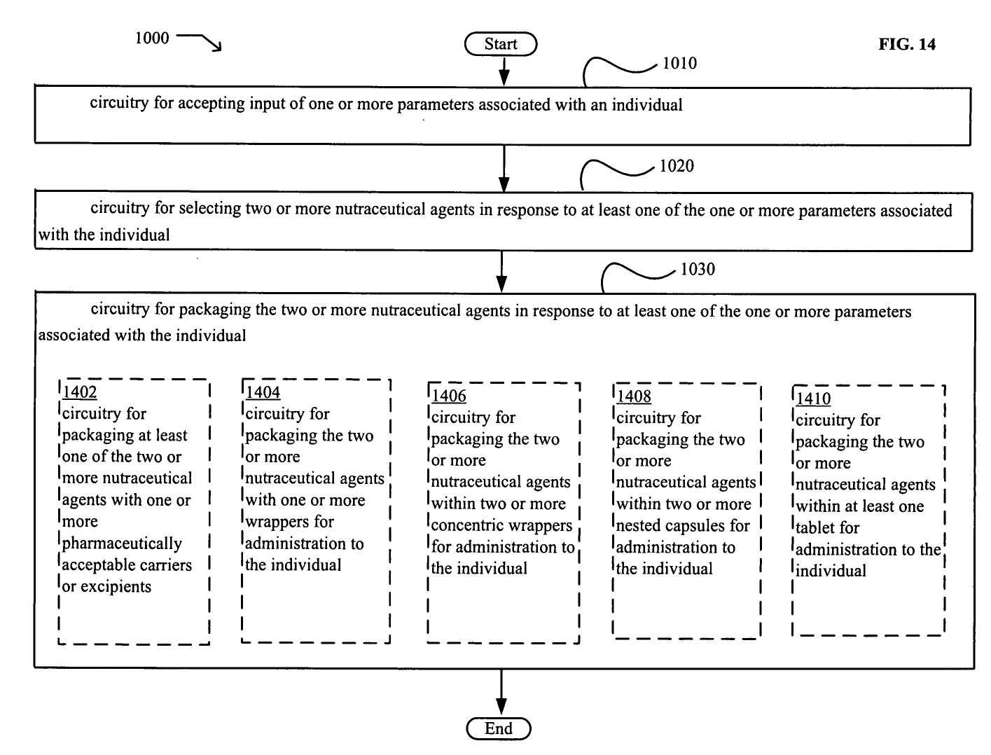

FIG. 14 illustrates alternative embodiments of system 1000 of FIG. 10. FIG. 14 illustrates example embodiments where the circuitry block 1030 may include at least one additional circuitry block 1402, circuitry block 1404, circuitry block 1406, circuitry block 1408, and/or circuitry block 1410.

At circuitry block 1402, the circuitry block 1030 may include circuitry for packaging at least one of the two or more nutraceutical agents with one or more pharmaceutically acceptable carriers or excipients. In some embodiments, one or more packaging units 114 may include circuitry for packaging at least one of the two or more nutraceutical agents 112 with one or more pharmaceutically acceptable carriers or excipients.

Nutraceutical agents 112 may be packaged through use of numerous known methods, such as conventional mixing, dissolving, granulating, dragee-making, levigating, emulsifying, encapsulating, entrapping or lyophilizing processes. In some embodiments, the nutraceutical agents 112 may be packaged in a manner that depends on the route that the nutraceutical agents 112 are to be administered to an individual 108.

In some embodiments, one or more nutraceutical agents 112 may be packaged with one or more solid or gel phase carriers or excipients. Examples of such carriers or excipients include, but are not limited to, croscarmellose sodium, povidone, microcrystalline cellulose, calcium carbonate, calcium phosphate, various sugars, starches, cellulose derivatives, gelatin, pregelatinized starch, polymers such as polyethylene glycols, lactose, lactose monohydrate, sucrose, talc, gelatin, agar, pectin, acacia, magnesium stearate, stearic acid and substantially any combination thereof. If a solid carrier is used, the one or more nutraceutical agents 112 may be tableted, placed in a hard gelatin capsule in powder or pellet form, packaged in the form of a troche or lozenge, and the like.

In some embodiments, one or more nutraceutical agents 112 may be packaged with a liquid carrier or excipient. Examples of such liquid carriers include syrup, peanut oil, olive oil, water, physiologically compatible buffers (i.e., Hanks solution and Ringers solution), physiological saline buffer, and the like. If a liquid carrier is used, the administration form may be in the form of a syrup, emulsion, drop, soft gelatin capsule, sterile injectable solution, suspension in an ampoule or vial, non-aqueous liquid suspension, and the like.

One or more nutraceutical agents 112 may be packaged in stable water-soluble dosage forms. For example, in some embodiments, an acceptable salt of one or more nutraceutical agents 112 may be dissolved in an aqueous solution of an organic or inorganic acid, such as 0.3M solution of succinic acid or citric acid. If a soluble salt form is not available, a nutraceutical agent 112 may be dissolved in a suitable cosolvent or combination of cosolvents. Examples of suitable cosolvents include, but are not limited to, alcohol, propylene glycol, polyethylene glycol 300, polysorbate 80, glycerin and the like in concentrations ranging from 0-60% of the total volume. In some embodiments, one or more nutraceutical agents 112 may be dissolved in DMSO and diluted with water. The administration form may also be in the form of a solution of a salt form of one or more nutraceutical agents 112 in an appropriate aqueous vehicle such as water or isotonic saline or dextrose solution.

In some embodiments, nutraceutical agents 112 that are hydrophobic may be packaged through use of a cosolvent system comprising benzyl alcohol, a nonpolar surfactant, a water-miscible organic polymer, and an aqueous phase. The cosolvent system may be the VPD co-solvent system. VPD is a solution of 3 percent weight/volume benzyl alcohol, 8 percent weight/volume of the nonpolar surfactant polysorbate 80, and 65 percent weight/volume polyethylene glycol 300, made up to volume in absolute ethanol. The VPD co-solvent system (VPD:5W) consists of VPD diluted 1:1 with a 5 percent dextrose in water solution. This co-solvent system dissolves hydrophobic pharmaceutical agents well, and itself produces low toxicity upon systemic administration. Accordingly, the co-solvent system may also be used to dissolve hydrophobic nutraceutical agents 112. The proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol (i.e., polyvinyl pyrrolidone; and other sugars or polysaccharides may substitute for dextrose). Many other delivery systems may be used to administer hydrophobic nutraceutical agents 112 as well. For example, liposomes and emulsions are well known examples of delivery vehicles or carriers for hydrophobic drugs that may also be used to deliver nutraceuticals. Certain organic solvents such as dimethysulfoxide also may be employed, although usually at the cost of greater toxicity.

Some nutraceutical agents 112 may be packaged as salts with compatible counter ions. Compatible salts may be formed with many acids, including hydrochloric, sulfuric, acetic, lactic, tartaric, malic, succinic, etc. Salts of nutraceutical agents 112 tend to be more soluble in aqueous or other protonic solvents than are the corresponding free-base forms.

Numerous carriers and excipients are known and are commercially available (i.e., The Merck Index, 13^{th} Edition, An Encyclopedia of Chemicals, Drugs, and Biologicals, Merck & Co. Inc., Whitehouse Station, N.J. 2001; Mosby's Drug Guide, Mosby, Inc., St. Louis, Mo. 2004; Remington: The Science and Practice of Pharmacy, 20^{th} Edition, Lippincott Williams & Wilkins, Philadelphia, Pa. 2000; Physicians' Desk Reference, 58^{th} Edition, Thompson, PDR, Montvale, N.J. 2004; U.S. Pat. Nos. 6,773,721; 7,053,107; 7,049,312 and Published U.S. Patent Application No. 20040224916; herein incorporated by reference).

At circuitry block 1404, the circuitry block 1030 may include circuitry for packaging the two or more nutraceutical agents with one or more wrappers in response to at least one of the one or more parameters associated with the individual. In some embodiments, one or more packaging units 114 may include circuitry for packaging the two or more nutraceutical agents 112 with one or more wrappers in response to at least one of the one or more parameters 106 associated with the individual 108. In some embodiments, two or more nutraceutical agents 112 may be packaged by wrapping the two or more nutraceutical agents 112 into a single administration form for administration to an individual 108. In some embodiments, the two or more nutraceutical agents 112 may be preformulated prior to being wrapped in one or more wrappers. For example, two or more nutraceutical agents 112 that are in tablet form may be wrapped into a single administration form. In other embodiments, the two or more nutraceutical agents 112 may be combined together and then wrapped in one or more wrappers. In other embodiments, two or more nutraceutical agents 112 may be combined together with a suitable carrier and then wrapped in one or more wrappers. Numerous materials may be used to wrap the two or more nutraceutical agents 112. Examples of such materials include, but are not limited to, polymers that include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methamethacrylate copolymers, shellac, and the like. Numerous water insoluble polymers may be used that include cellulose derivatives (i.e., ethylcellulose), polyvinyl acetate, neutral copolymers based on ethyl acrylate and methylmethacrylate, copolymers of acrylic and methacrylic acid esters with quaternary ammonium groups, and the like. In some embodiments, polymers used in forming the wrappers may be plasticized. Examples of plasticizers that may be used to plasticize the wrappers include, but are not limited to, triacetin, tributyl citrate, triethyl citrate, acetyl tri-n-butyl citrate diethyl phthalate, castor oil, dibutyl sebacate, acetylated monoglycerides, and the like and/or substantially any combination thereof. In some embodiments, the plasticizer may be present at about 3 to 30 weight percent and more typically about 10 to 25 weight percent based on the polymer to which the plasticizer is added. The type of plasticizer and its content depends on the polymer or polymers, nature of the coating system. In some embodiments, water-soluble nonionic polysaccharide derivatives may be used to wrap one or more nutraceutical agents 112. For example, hydroxypropylmethylcellulose, hydroxypropylcellulose, and/or sodium carboxymethylcellulose may be used. Such polymers form coatings that quickly dissolve in water and have a high permeability. Accordingly, in some embodiments, such polymers may be used for rapid release of one or more nutraceutical agents 112 that are wrapped in such a wrapper following administration to an individual 108. In some embodiments, one or more nutraceutical agents 112 may be wrapped in a wrapper that provides for sustained release of the one or more nutraceutical agents 112. For example, one or more nutraceutical agents 112 may be released continuously over twelve hours through use of wrappers constructed from ethyl cellulose and an ethyl acrylate-methyl methacrylate-ethyl trimethylammoniumchloride methacrylate copolymer as the release controlling wrapper. Methods and materials that may be used to prepare wrappers are known in the art and are commercially available (i.e., Rohm Pharma, Piscataway, N.J.; U.S. Pat. Nos. 6,656,507; 7,048,945; 7,056,951; hereby incorporated by reference).

In some embodiments, one wrapper may be used to wrap two or more nutraceutical agents 112 into an administration form. For example, the two or more nutraceutical agents 112 may be combined together and then wrapped into an administration form in one wrapper for release at the same time following administration to an individual 108. In other embodiments, one continuous wrapper may be used to wrap the two or more nutraceutical agents 112 into an administration form in which the two or more nutraceutical agents 112 are separated from each other. For example, in some embodiments, one of the two or more nutraceutical agents 112 may be covered with a continuous wrapper to form a core and then a second nutraceutical agent 112 may be wrapped around the core with the continuous wrapper to form an administration form. This process may be repeated with multiple nutraceutical agents 112 to form a multilayered administration form in which the multiple nutraceutical agents 112 are separated from each other. In some embodiments, such a configuration provides for the release of nutraceutical agents 112 from the administration form at different times and/or at different sites associated with an individual 108 to which the administration form is administered. In some embodiments, two or more nutraceutical agents 112 are wrapped into an administration form together and additional nutraceutical agents 112 are wrapped into the administration form in separate layers. Accordingly, nutraceutical agents 112 may be oriented in the administration form to be released from the administration form at the same time and/or site or such that they are released at different times and/or sites. Examples of such sites include, but are not limited to, the mouth, esophagus, stomach, duodenum, small intestine, large intestine, and the rectum.

At circuitry block 1406, the circuitry block 1030 may include circuitry for packaging the two or more nutraceutical agents within two or more concentric wrappers in response to at least one of the one or more parameters associated with the individual. In some embodiments, one or more packaging units 114 may include circuitry for packaging the two or more nutraceutical agents 112 within two or more concentric wrappers in response to at least one of the one or more parameters 106 associated with the individual 108. In some embodiments, two or more nutraceutical agents 112 may be packaged by wrapping the two or more nutraceutical agents 112 within two or more wrappers to form an administration form. In some embodiments, the same type of material is used to form the two or more wrappers in the administration form. In some embodiments, different types of material are used as wrappers to form the administration form. For example, an outer wrapper may be selected to dissolve rapidly and release one or more nutraceutical agents 112 soon after administration of the administration form to the individual 108 while an inner wrapper may be selected to release one or more nutraceutical agents 112 at a later time and/or at a different site associated with an individual 108. Accordingly, in some embodiments, multiple nutraceutical agents 112 may be packaged into the same administration form for release at different times and at different sites following administration of the administration form to an individual 108. In some embodiments, the nutraceutical agents 112 may be the same to provide for continuous dosing of an individual 108. In some embodiments, the nutraceutical agents 112 may be different to provide for dosing of an individual 108 with different nutraceutical agents 112. In some embodiments, some of the nutraceutical agents 112 may be the same to provide for continuous dosing of an individual 108 and others may be different to provide for dosing of an individual 108 with different nutraceutical agents 112. Accordingly, numerous combinations of nutraceutical agents 112 and wrappers may be assembled into an administration form.

At circuitry block 1408, the circuitry block 1030 may include circuitry for packaging the two or more nutraceutical agents within two or more nested capsules in response to at least one of the one or more parameters associated with the individual. In some embodiments, one or more packaging units 114 may include circuitry for packaging the two or more nutraceutical agents 112 within two or more nested capsules in response to at least one of the one or more parameters 106 associated with the individual 108. In some embodiments, two or more nutraceutical agents 112 may be packaged into an administration form through use of nested capsules. In some embodiments, a first nutraceutical agent 112 may be packaged in a first capsule and a second nutraceutical agent 112 may be packaged in a second capsule in which the first capsule is included to create an administration form having nested capsules. Accordingly, administration forms may be constructed that include two or more nested capsules. In some embodiments, such administration forms may include two or more nutraceutical agents 112. In other embodiments, such administration forms may include one type of nutraceutical agent 112 that is contained within multiple capsules of the administration form and one or more types of different nutraceutical agents 112 that are also contained within the capsules included within the administration form. In some embodiments, the material used to construct the individual capsules of a single administration form is the same. In some embodiments, the material used to construct the individual capsules of a single administration form is different. In some embodiments, the material used to construct some of the individual capsules of a single administration form may be the same while the material used to construct other individual capsules of the single administration form may be different. Accordingly, through selection of materials used to construct the individual capsules contained in an administration form, two or more nutraceutical agents 112 may be released from one administration form at one or more times and/or at one or more sites associated with the individual 108. For example, as with wrapping materials described herein, materials may be selected for constructing capsules that release one or more nutraceutical agents 112 at a site associated with an individual 108. Examples of such sites include, but are not limited to, the mouth, esophagus, stomach, duodenum, small intestine, large intestine, and the rectum.

At circuitry block 1410, the circuitry block 1030 may include circuitry for packaging the two or more nutraceutical agents within at least one tablet in response to at least one of the one of more parameters associated with the individual. In some embodiments, one or more packaging units 114 may include circuitry for packaging the two or more nutraceutical agents 112 within at least one tablet in response to at least one of the one or more parameters 106 associated with the individual 108. In some embodiments, two or more nutraceutical agents 112 may be selected in response to one or more parameters 106 associated with an individual 108 and packaged into at least one table. Methods to package two or more nutraceutical agents 112 into at least one tablet for administration to an individual 108 are known (i.e., Published U.S. Patent Application Nos. 20040224916 and 20050013863; and U.S. Pat. Nos. 5,490,962; 6,280,771; herein incorporated by reference). Accordingly, in some embodiments, two or more nutraceutical agents 112 may be packaged into a tablet such that the two or more nutraceutical agents 112 are released at the same or different times following administration of the tablet to an individual 108. In other embodiments, two or more nutraceutical agents 112 may be packaged into a tablet such that the two or more nutraceutical agents 112 are released at the same or different sites associated with an individual 108 following administration of the tablet to an individual 108. In other embodiments, two or more nutraceutical agents 112 may be packaged into a tablet such that the two or more nutraceutical agents 112 are released at the same or different times and at the same or different sites associated with an individual 108 following administration of the tablet to the individual 108.

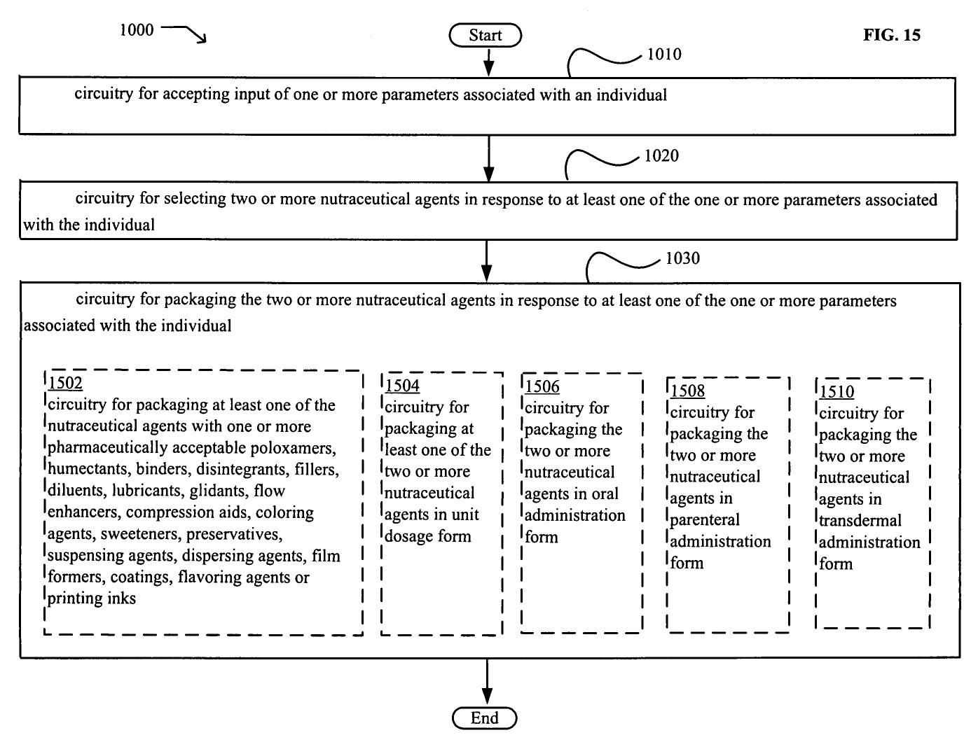

FIG. 15 illustrates alternative embodiments of system 1000 of FIG. 10. FIG. 15 illustrates example embodiments where circuitry block 1030 may include at least one additional circuitry block 1502, circuitry block 1504, circuitry block 1506, circuitry block 1508, and/or circuitry block 1510.

At circuitry block 1502, the circuitry block 1030 may include circuitry for packaging at least one of the nutraceutical agents with one or more pharmaceutically acceptable poloxamers, humectants, binders, disintegrants, fillers, diluents, lubricants, glidants, flow enhancers, compression aids, coloring agents, sweeteners, preservatives, suspensing agents, dispersing agents, film formers, coatings, flavoring agents or printing inks. In some embodiments, one or more packaging units 114 may include circuitry for packaging at least one of the nutraceutical agents 112 with one or more pharmaceutically acceptable poloxamers, humectants, binders, disintegrants, fillers, diluents, lubricants, glidants, flow enhancers, compression aids, coloring agents, sweeteners, preservatives, suspending agents, dispersing agents, film formers, coatings, flavoring agents or printing inks.

At circuitry block 1504, the circuitry block 1030 may include circuitry for packaging at least one of the two or more nutraceutical agents in unit dosage form. In some embodiments, one or more packaging units 114 may include circuitry for packaging at least one of the two or more nutraceutical agents 112 in unit dosage form.

The term “unit dosage form” refers to one or more amounts of one or more nutraceutical agents 112 that are suitable as unitary dosages for individuals, such as human and non-human individuals, with each unit containing a predetermined quantity of at least one nutraceutical agent 112 calculated to produce a desired effect, such as a therapeutic effect, in association with one or more suitable pharmaceutical carriers. Such unit dosage forms may be packaged in numerous configurations that include, but are not limited to, tablets, capsules, ampoules, and other administration forms known in the art and described herein. In some embodiments, two or more unit dosage forms of one or more nutraceutical agents 112 may be packaged into an administration form. For example, in some embodiments, two unit dosage forms may be wrapped into an administration form through use of a continuous wrapper such that they are released at different times following administration to an individual 108. In such an example, two unit dosage forms are included within one administration form. Accordingly, numerous combinations of nutraceutical agents 112 and unit dosage forms may be included within an administration form.

At circuitry block 1506, the circuitry block 1030 may include circuitry for packaging the two or more nutraceutical agents in oral administration form. In some embodiments, one or more packaging units 114 may include circuitry for packaging the two or more nutraceutical agents 112 in oral administration form.

For oral administration, one or more nutraceutical agents 112 may be packaged into an oral administration form by combining the one or more nutraceutical agents 112 with pharmaceutically acceptable carriers that are well known in the art. Such carriers allow the one or more nutraceutical agents 112 to be formulated as tablets, pills, dragees, capsules, liquids, gels, syrups, slurries, suspensions and the like, for oral ingestion by an individual 108. Oral administration forms can be obtained by combining the one or more nutraceutical agents 112 with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of nutraceutical agents 112.

Oral administration forms may include push-fit capsules made of gelatin, as well as soft, sealed capsules made of gelatin and a plasticizer, such as glycerol or sorbitol. The push-fit capsules can contain one or more nutraceutical agents 112 in admixture with a filler such as lactose, binders such as starches, and/or lubricants such as talc or magnesium stearate and, optionally, stabilizers. In soft capsules, the nutraceutical agents 112 may be dissolved or suspended in suitable liquids, such as fatty oils, liquid paraffin, or liquid polyethylene glycols. In addition, stabilizers may be added. All oral dosage forms may be prepared in dosages suitable for such administration. For buccal administration, the nutraceutical agents 112 may take the form of tablets or lozenges formulated in a conventional manner.

At circuitry block 1508, the circuitry block 1030 may include circuitry for packaging the two or more nutraceutical agents in parenteral administration form. In some embodiments, one or more packaging units 114 may include circuitry for packaging the two or more nutraceutical agents 112 in parenteral administration form.

The one or more nutraceutical agents 112 may be formulated for parenteral administration by injection (i.e., bolus injection or continuous infusion). Formulations for injection may be presented in unit dosage form (i.e., in ampoules or in multi-dose containers) with an added preservative. The administration forms may take such forms as suspensions, solutions or emulsions in oily or aqueous vehicles, and may contain formulatory agents such as suspending, stabilizing and/or dispersing agents. Administration forms for parenteral administration may include aqueous solutions of the one or more nutraceutical agents 112 in water-soluble form. In some embodiments, the one or more nutraceutical agents 112 may be formulated in physiologically compatible buffers that include Hanks solution, Ringers solution, physiological saline buffer, and the like. Additionally, suspensions of the one or more nutraceutical agents 112 may be prepared as appropriate oily injection suspensions. Suitable lipophilic solvents include fatty oils such as sesame oil, or synthetic fatty acid esters, such as ethyl oleate or triglycerides, or liposomes. Aqueous injection suspensions may include substances which increase the viscosity of the suspension, such as sodium carboxymethyl cellulose, sorbitol, or dextran. Optionally, the suspension may also contain suitable stabilizers or agents which increase the solubility of the one or more nutraceutical agents 112 to allow for the preparation of highly concentrated solutions.

At circuitry block 1510, the circuitry block 1030 may include circuitry for packaging the two or more nutraceutical agents in transdermal administration form. In some embodiments, one or more packaging units 114 may include circuitry for packaging the two or more nutraceutical agents 112 in transdermal administration form. For transdermal, including transmucosal, administration of the one or more nutraceutical agents 112, penetrants appropriate to the barrier or barriers to be permeated may be used in the formulation. Briefly, in some embodiments, a transdermal administration form may include an ethoxylated lipid, an alcohol mixed with the ethoxylated lipid to form a penetration enhancer, an aqueous adjuvant mixed with the penetration enhancer, and a delivered nutraceutical agent 112 mixed with the aqueous adjuvant and the penetration enhancer. In some embodiments, the aqueous adjuvant is a plant extract from the family of Liliaceae Liliaceae. In some embodiments, the ethoxylated lipid is a vegetable oil or animal oil having at least 20 ethoxylations per molecule. In other embodiments, about 0.1 percent to 40.0 percent by weight or volume is ethoxylated lipid. Other embodiments may include a transdermal delivery system that includes about 0.1 percent to 15 percent by weight or volume of alcohol or where about 0.1 percent to 85 percent by weight or volume is Aloe Vera. Numerous transdermal administration forms are known and have been described (i.e., U.S. Pat. Nos. 5,820,876; 7,045,145; 6,946,144; incorporated herein by reference).

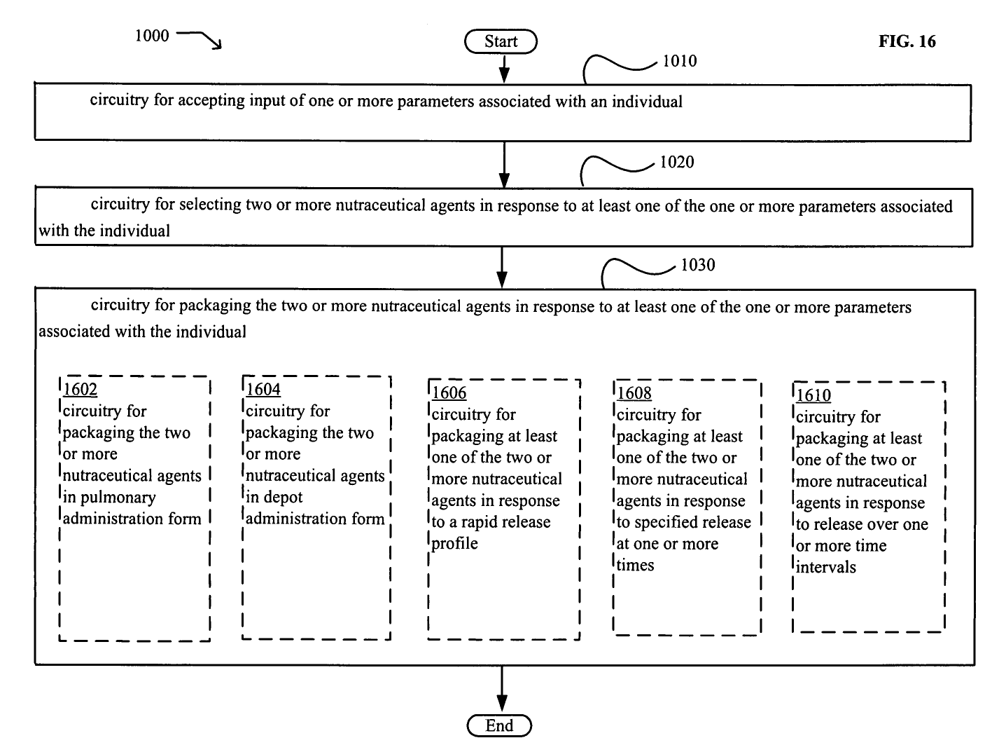

FIG. 16 illustrates alternative embodiments of system 1000 of FIG. 10. FIG. 16 illustrates example embodiments where circuitry block 1030 may include at least one additional circuitry block 1602, circuitry block 1604, circuitry block 1606, circuitry block 1608, and/or circuitry block 1610.

At circuitry block 1602, the circuitry block 1030 may include circuitry for packaging the two or more nutraceutical agents in pulmonary administration form. In some embodiments, one or more packaging units 114 may include circuitry for packaging the two or more nutraceutical agents 112 in pulmonary administration form. For pulmonary administration, the one or more nutraceutical agents 112 may be delivered in the form of an aerosol spray from pressurized packs or a nebuliser, with the use of a suitable propellant (i.e., dichlorodifluoromethane, trichlorofluoromethane, dichlorotetrafluoroethane, carbon dioxide or other suitable gas). In the case of a pressurized aerosol, the dosage unit may be determined by providing a valve to deliver a metered amount of the one or more nutraceutical agents 112. Capsules and cartridges for use in an inhaler or insufflator may be formulated to contain a powder mix of the one or more nutraceutical agents 112 and a suitable powder base such as lactose or starch. Methods and materials that may be used to package one or more nutraceutical agents 112 in pulmonary administration form are known and have been described (i.e., U.S. Pat. Nos. 6,921,527; 6,838,076; 6,565,841; 6,451,286; 6,169,068; 5,993,783; 5,780,014; 5,719,123; 5,354,934; 5,284,656; 5,006,343; hereby incorporated by reference).

At circuitry block 1604, the circuitry block 1030 may include circuitry for packaging the two or more nutraceutical agents in depot administration form. In some embodiments, one or more packaging units 114 may include circuitry for packaging the two or more nutraceutical agents 112 in depot administration form. In some embodiments, depot administration forms may be administered by implantation (i.e., subcutaneously, intramuscularly, intramuscular injection, subtenon, intravitreal injection). Accordingly, for example, the one or more nutraceutical agents 112 may be packaged with suitable polymeric or hydrophobic materials, ion exchange resins, and the like. Methods and materials that may be used to package nutraceutical agents 112 in depot administration form are known and are commercially available (i.e., U.S. Pat. Nos. 6,773,714; 6,630,155; 6,565,874; 5,945,115; herein incorporated by reference).

At circuitry block 1606, the circuitry block 1030 may include circuitry for packaging at least one of the two or more nutraceutical agents in response to a rapid release profile. In some embodiments, one or more packaging units 114 may include circuitry for packaging at least one of the two or more nutraceutical agents 112 in response to a rapid release profile. In some embodiments, water-soluble nonionic polysaccharide derivatives may be used to package one or more nutraceutical agents 112. For example, hydroxypropylmethylcellulose, hydroxypropylcellulose, and/or sodium carboxymethylcellulose may be used. Such polymers form coatings that quickly dissolve in water and have a high permeability. Accordingly, in some embodiments, such polymers may be used for rapid release of one or more nutraceutical agents 112 that are packaged in such materials following administration to an individual 108. Numerous rapid release formulations are known and have been described (i.e., U.S. Pat. No. 6,979,463; herein incorporated by reference).

At circuitry block 1608, the circuitry block 1030 may include circuitry for packaging at least one of the two or more nutraceutical agents in response to specified release at one or more times. In some embodiments, one or more packaging units 114 may include circuitry for packaging at least one of the two or more nutraceutical agents 112 in response to specified release at one or more times. In some embodiments, one or more nutraceutical agents 112 may be packaged so that they are released from an administration form at one or more times following administration to an individual 108. In some embodiments, one or more nutraceutical agents 112 may be released at one or more times following administration to maintain the dosage of the one or more nutraceutical agents 112 at or above a certain concentration. Accordingly, in some embodiments, the concentration of one nutraceutical agent 112 may be maintained over a period of time in association with an individual 108. In other embodiments, the concentration of more than one nutraceutical agent 112 may be maintained over a period of time in association with an individual 108. In some embodiments, one or more nutraceutical agents 112 may be packaged to be released in anticipation of an event, such as a long airplane flight. For example, in some embodiments, one or more nutraceutical agents 112 that induce sleep may be packaged into an administration form so that an individual 108 to whom the administration form is administered will fall asleep at a precalculated time on an airplane during a long flight. In other embodiments, one or more nutraceutical agents 112 may be packaged into an administration form such that an individual 108 to whom the administration form is administered will not fall asleep during a long meeting or presentation. For example, an administration form may be prepared with non-drowsy versions of one or more nutraceutical agents 112. Numerous methods may be used to package one or more nutraceutical agents 112 for release at one or more times. For example, in some embodiments, one or more nutraceutical agents 112 may be wrapped into an administration form through methods described herein. In such examples, the time of release of the one or more nutraceutical agents 112 from the administration form may be controlled through selection of wrappers used to formulate the administration form. For example, a thick wrapper may be used to delay release while a thin wrapper may be used to expedite release of the one or more nutraceutical agents 112 from the administration form. In other embodiments, one or more wrappers may be selected that are made of material that is more or less resistant to degradation when administered to an individual 108. Accordingly, materials having various chemical and physical properties may be selected to produce administration forms that release one or more nutraceutical agents 112 at one or more times.

At circuitry block 1610, the circuitry block 1030 may include circuitry for packaging at least one of the two or more nutraceutical agents in response to release over one or more time intervals. In some embodiments, one or more packaging units 114 may include circuitry for packaging at least one of the two or more nutraceutical agents 112 in response to release over one or more time intervals.

In some embodiments, one or more nutraceutical agents 112 may be packaged so that they are released from an administration form over one or more time intervals following administration to an individual 108. In some embodiments, one or more nutraceutical agents 112 may be released over one or more times following administration to maintain the dosage of the one or more nutraceutical agents 112 at or above a certain concentration. Accordingly, in some embodiments, the concentration of one nutraceutical agent 112 may be maintained over a period of time in association with an individual 108. In other embodiments, the concentration of more than one nutraceutical agent 112 may be maintained over a period of time in association with an individual 108. In some embodiments, one or more nutraceutical agents 112 may be packaged to be released over one or more time intervals in anticipation of an event, such as a long airplane flight, that may occur during the one or more time intervals. For example, in some embodiments, one or more nutraceutical agents 112 that induce sleep may be packaged into an administration form so that they are released during the time interval in which an individual 108 to whom the administration form is administered is on an airplane. Numerous methods may be used to package one or more nutraceutical agents 112 for release over one or more time intervals. For example, in some embodiments, one or more nutraceutical agents 112 may be wrapped into an administration form through methods described herein. In such examples, the time of release of the one or more nutraceutical agents 112 from the administration form may be controlled through selection of wrappers used to formulate the administration form. For example, a thick wrapper may be used to delay release while a thin wrapper may be used to expedite release of the one or more nutraceutical agents 112 from the administration form. In other embodiments, one or more wrappers may be selected that are made of material that is more or less resistant to degradation when administered to an individual 108. In other embodiments, controlled-release formulations may be acquired and then packaged for release over one or more time intervals.

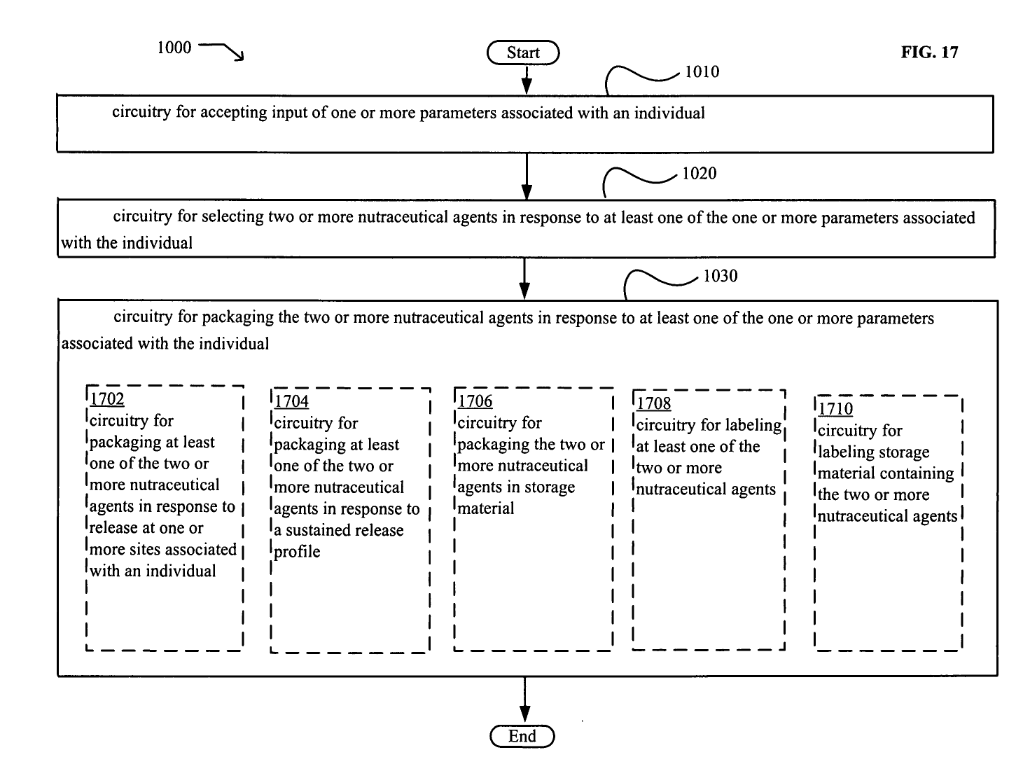

FIG. 17 illustrates alternative embodiments of system 1000 of FIG. 10. FIG. 17 illustrates example embodiments where circuitry block 1030 may include at least one additional circuitry block 1702, circuitry block 1704, circuitry block 1706, circuitry block 1708, and/or circuitry block 1710.

At circuitry block 1702, the circuitry block 1030 may include circuitry for packaging at least one of the two or more nutraceutical agents in response to release at one or more sites associated with an individual. In some embodiments, one or more packaging units 114 may include circuitry for packaging at least one of the two or more nutraceutical agents 112 in response to release at one or more sites associated with an individual 108. One or more nutraceutical agents 112 may be packaged for administration to numerous sites that are associated with an individual 108. Examples of such sites include, but are not limited to, the eyes, ears, nose, skin, mouth, stomach, intestine, rectum, vagina, vascular system, pulmonary system, gastrointestinal system, urinary system and lymphatic system. Accordingly, in some embodiments, release of one or more nutraceutical agents 112 from an administration form at one or more sites associated with an individual 108 may be controlled through selection of materials that degrade under conditions present at the desired site of release. For example, for release in the stomach, one or more nutraceutical agents 112 may be packaged into an administration form that degrades when exposed to acidic conditions. In other examples, one or more nutraceutical agents 112 may be released in the gastrointestinal tract by preparing an administration form that is acid resistant but that degrades under basic conditions. Numerous methods are known that may be used to release one or more nutraceutical agents 112 at one or more sites associated with an individual 108.

At circuitry block 1704, the circuitry block 1030 may include circuitry for packaging at least one of the two or more nutraceutical agents in response to a sustained release profile. In some embodiments, one or more packaging units 114 may include circuitry for packaging at least one of the two or more nutraceutical agents 112 in response to a sustained release profile. In some embodiments, one or more nutraceutical agents 112 may be packaged with a carrier that may include a time-delay or time-release material known in the art, such as glyceryl monostearate or glyceryl distearate alone or with a wax, ethylcellulose, hydroxypropylmethylcellulose, methylmethacrylate and the like. Additionally, in some embodiments, one or more nutraceutical agents 112 may be administered using a sustained-release system, such as semipermeable matrices of solid hydrophobic polymers containing the one or more nutraceutical agents 112. Various sustained-release materials are known and have been described. For example, sustained-release capsules may, depending on their chemical composition, release one or more nutraceutical agents 112 for a few weeks up to over 100 days. Numerous additional sustained-release formulations are known and have been described (i.e., U.S. Pat. Nos. 7,041,670; 7,041,317; 6,709,676; herein incorporated by reference).

At circuitry block 1706, the circuitry block 1030 may include circuitry for packaging the two or more nutraceutical agents in storage material. In some embodiments, one or more packaging units 114 may include circuitry for packaging the two or more nutraceutical agents 112 in storage material. Two or more nutraceutical agents 112 may be packaged in numerous types of storage material. Examples of storage material include, but are not limited to, containers, boxes, ampoules, vials, syringes, and the like. In some embodiments, storage material includes advertising. In some embodiments, storage material includes instructions for administration. Such instructions may include time for administration, route of administration, the name of the individual 108 to whom the two or more nutraceutical agents 112 are to be administered, the identity of the two or more nutraceutical agents 112, the dosage of the two or more nutraceutical agents 112, appropriate buffers for suspension of the two or more nutraceutical agents 112, the source of the two or more nutraceutical agents 112, the date when the two or more nutraceutical agents 112 were packaged, the date when the two or more nutraceutical agents 112 were manufactured, the expiration date of the two or more nutraceutical agents 112, and the like.

At circuitry block 1708, the circuitry block 1030 may include circuitry for labeling at least one of the two or more nutraceutical agents. In some embodiments, one or more packaging units 114 may include circuitry for labeling at least one of the two or more nutraceutical agents 112. In some embodiments, one or more packaging units 114 may place a label directly on at least one of the two or more nutraceutical agents 112. Numerous methods may be used to label at least one of the two or more nutraceutical agents 112. For example, in some embodiments, one or more packaging units 114 may stamp an indented label into at least one of the two or more nutraceutical agents 112. In some embodiments, one or more packaging units 114 may stamp a label onto at least one of the two or more nutraceutical agents 112 through use of one or more edible dyes. Such labels may include numerous types of information. For example, such labels may indicate the manufacturer of at least one of the two or more nutraceutical agents 112, the date of manufacture, the date of packaging, the dosage, the route of administration, and the like. Such labels may be in any substantially any language. In some embodiments, at least one label may be a bar code.

At circuitry block 1710, the circuitry block 1030 may include circuitry for labeling storage material containing the two or more nutraceutical agents. In some embodiments, one or more packaging units 114 may include circuitry for labeling storage material containing the two or more nutraceutical agents 112. In some embodiments, storage material may be labeled with advertising. In some embodiments, storage material may be labeled with instructions for administration. Such instructions may include time for administration, route of administration, the name of the individual 108 to whom the two or more nutraceutical agents 112 are to be administered, the identity of the two or more nutraceutical agents 112, the dosage of the two or more nutraceutical agents 112, appropriate buffers for suspension of the two or more nutraceutical agents 112, the source of the two or more nutraceutical agents 112, the date when the two or more nutraceutical agents 112 were packaged, the date when the two or more nutraceutical agents 112 were manufactured, the expiration date of the two or more nutraceutical agents 112, and the like.

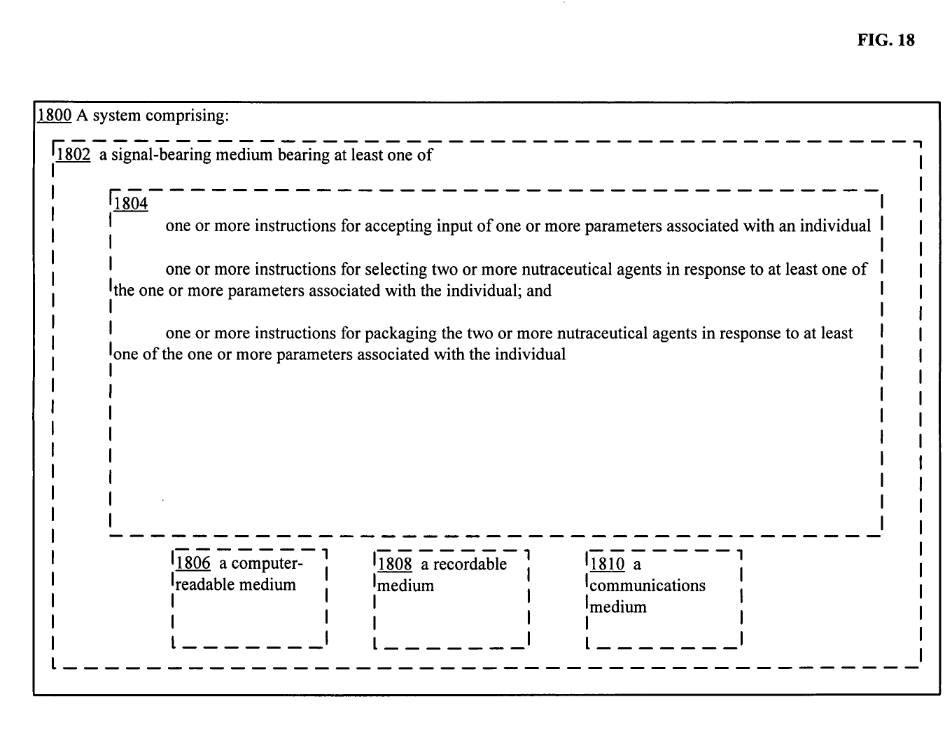

FIG. 18 illustrates a partial view of a system 1800 that includes a computer program 1804 for executing a computer process on a computing device. An embodiment of the system 1800 is provided using a signal-bearing medium 1802 bearing at least one of one or more instructions for accepting input of one or more parameters associated with an individual, one or more instructions for selecting two or more nutraceutical agents in response to at least one of the one or more parameters associated with the individual; and one or more instructions for packaging the two or more nutraceutical agents in response to at least one of the one or more parameters associated with the individual. The one or more instructions may be, for example, computer executable and/or logic-implemented instructions. In some embodiments, the signal-bearing medium 1802 may include a computer-readable medium 1806. In some embodiments, the signal bearing medium 1802 may include a recordable medium 1808. In some embodiments, the signal bearing medium 1802 may include a communications medium 1810.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various embodiments described herein can be implemented, individually and/or collectively, by various types of electro-mechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electronics systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, hovercraft, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a voice-over IP system, etc.), (f) a business entity (e.g., an Internet Service Provider (ISP) entity such as Comcast Cable, Quest, Southwestern Bell, etc), or (g) a wired/wireless services entity such as Sprint, Cingular, Nextel, etc.), etc.

Although user 120 is shown/described herein as a single illustrated figure, those skilled in the art will appreciate that a user 120 may be representative of a human user, a robotic user 120 (e.g., computational entity), and/or substantially any combination thereof (e.g., a user 120 may be assisted by one or more robotic agents). In addition, a user 120 as set forth herein, although shown as a single entity may in fact be composed of two or more entities. Those skilled in the art will appreciate that, in general, the same may be said of “sender” and/or other entity-oriented terms as such terms are used herein.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

All publications, patents and patent applications cited herein are incorporated herein by reference. The foregoing specification has been described in relation to certain embodiments thereof, and many details have been set forth for purposes of illustration, however, it will be apparent to those skilled in the art that the invention is susceptible to additional embodiments and that certain of the details described herein may be varied considerably without departing from the basic principles of the invention.

What is claimed is:

1.-40. (canceled)

41. A system comprising:
circuitry for accepting input of one or more parameters associated with an individual;
circuitry for selecting two or more nutraceutical agents in response to at least one of the one or more parameters associated with the individual; and
circuitry for packaging the two or more nutraceutical agents in response to at least one of the one or more parameters associated with the individual.

42. The system of claim 41, wherein the circuitry for accepting input of one or more parameters associated with an individual comprises:
circuitry for accepting the one or more parameters associated with a human individual.

43. (canceled)

44. The system of claim 41, wherein the circuitry for accepting input of one or more parameters associated with an individual comprises:
circuitry for accepting the one or more parameters associated with a physician input.

45. (canceled)

46. The system of claim 41, wherein the circuitry for accepting input of one or more parameters associated with an individual comprises:
circuitry for accepting the one or more parameters associated with a pharmacist input.

47. The system of claim 41, wherein the circuitry for accepting input of one or more parameters associated with an individual comprises:
circuitry for accepting the one or more parameters associated with a patient input.

48. The system of claim 41, wherein the circuitry for accepting input of one or more parameters associated with an individual comprises:
circuitry for accepting the one or more parameters associated with a machine input.

49. (canceled)

50. The system of claim 41, wherein the circuitry for selecting two or more nutraceutical agents in response to at least one of the one or more parameters associated with the individual comprises:
circuitry for selecting at least one of the two or more nutraceutical agents in response to at least one condition specifically associated with the individual.

51. (canceled)

52. The system of claim 41, wherein the circuitry for selecting two or more nutraceutical agents in response to at least one of the one or more parameters associated with the individual comprises:
circuitry for selecting at least one of the two or more nutraceutical agents in response to at least one dosage specifically associated with the individual.

53. The system of claim 41, wherein the circuitry for selecting two or more nutraceutical agents in response to at least one of the one or more parameters associated with the individual comprises:
circuitry for selecting the two or more nutraceutical agents in response to dosage of at least one of the two or more nutraceutical agents.

54. The system of claim 41, wherein the circuitry for selecting two or more nutraceutical agents in response to at least one of the one or more parameters associated with the individual comprises:
circuitry for selecting at least one of the two or more nutraceutical agents in response to at least one time of administration.

55. The system of claim 41, wherein the circuitry for selecting two or more nutraceutical agents in response to at least one of the one or more parameters associated with the individual comprises:
circuitry for selecting at least one of the two or more nutraceutical agents in response to two or more times of administration within a time period.

56. The system of claim 41, wherein the circuitry for selecting two or more nutraceutical agents in response to at least one of the one or more parameters associated with the individual comprises:
circuitry for selecting at least one of the two or more nutraceutical agents in response to one or more sites of administration associated with the individual.

57.-58. (canceled)

59. The system of claim 41, wherein the circuitry for selecting two or more nutraceutical agents in response to at least one of the one or more parameters associated with the individual comprises:
circuitry for selecting at least one of the two or more nutraceutical agents in response to cost associated with at least one of the two or more nutraceutical agents.

60. The system of claim 41, wherein the circuitry for selecting two or more nutraceutical agents in response to at least one of the one or more parameters associated with the individual comprises:
circuitry for selecting at least one of the two or more nutraceutical agents in response to compatibility of at least one of the nutraceutical agents with another of the two or more nutraceutical agents.

61. The system of claim 41, wherein the circuitry for packaging the two or more nutraceutical agents in response to at least one of the one or more parameters associated with the individual comprises:
circuitry for packaging at least one of the two or more nutraceutical agents with one or more pharmaceutically acceptable carriers or excipients.

62. The system of claim 41, wherein the circuitry for packaging the two or more nutraceutical agents in response to at least one of the one or more parameters associated with the individual comprises:
circuitry for packaging the two or more nutraceutical agents with one or more wrappers in response to at least one of the one or more parameters associated with the individual.

63. (canceled)

64. The system of claim 41, wherein the circuitry for packaging the two or more nutraceutical agents in response to at least one of the one or more parameters associated with the individual comprises:
circuitry for packaging the two or more nutraceutical agents within two or more nested capsules in response to at least one of the one or more parameters associated with the individual.

65. The system of claim 41, wherein the circuitry for packaging the two or more nutraceutical agents in response to at least one of the one or more parameters associated with the individual comprises:
circuitry for packaging the two or more nutraceutical agents within at least one tablet in response to at least one of the one of more parameters associated with the individual.

66. (canceled)

67. The system of claim 41, wherein the circuitry for packaging the two or more nutraceutical agents in response to at least one of the one or more parameters associated with the individual comprises:
circuitry for packaging at least one of the two or more nutraceutical agents in unit dosage form.

68. The system of claim 41, wherein the circuitry for packaging the two or more nutraceutical agents in response to at least one of the one or more parameters associated with the individual comprises:
circuitry for packaging the two or more nutraceutical agents in oral administration form.

69.-72. (canceled)

73. The system of claim 41, wherein the circuitry for packaging the two or more nutraceutical agents in response to at least one of the one or more parameters associated with the individual comprises:
circuitry for packaging at least one of the two or more nutraceutical agents in response to a rapid release profile.

74. The system of claim 41, wherein the circuitry for packaging the two or more nutraceutical agents in response to at least one of the one or more parameters associated with the individual comprises:
circuitry for packaging at least one of the two or more nutraceutical agents in response to specified release at one or more times.

75.-77. (canceled)

78. The system of claim 41, wherein the circuitry for packaging the two or more nutraceutical agents in response to at least one of the one or more parameters associated with the individual comprises:
circuitry for packaging the two or more nutraceutical agents in storage material.

79. (canceled)

80. The system of claim 41, wherein the circuitry for packaging the two or more nutraceutical agents in response to at least one of the one or more parameters associated with the individual comprises:
circuitry for labeling storage material containing the two or more nutraceutical agents.

81. A system comprising:
means for accepting input of one or more parameters associated with an individual;
means for selecting two or more nutraceutical agents in response to at least one of the one or more parameters associated with the individual; and
means for packaging the two or more nutraceutical agents in response to at least one of the one or more parameters associated with the individual.

82. A system comprising:
a signal-bearing medium bearing:
one or more instructions for accepting input of one or more parameters associated with an individual;
one or more instructions for selecting two or more nutraceutical agents in response to at least one of the one or more parameters associated with the individual; and
one or more instructions for packaging the two or more nutraceutical agents in response to at least one of the one or more parameters associated with the individual.

83. The system of claim 82, wherein the signal-bearing medium includes a computer-readable medium.

84. The system of claim 82, wherein the signal-bearing medium includes a recordable medium.

85. The system of claim 82, wherein the signal-bearing medium includes a communications medium.
